(19) United States
(12) Patent Application Publication
(10) Pub. No.: US 2024/0216401 A1
(43) Pub. Date: Jul. 4, 2024

(54) COMBINATION THERAPY BY USING AKR1C3-ACTIVATED COMPOUND WITH IMMUNE CHECKPOINT INHIBITOR

(71) Applicant: OBI Pharma, Inc., Taipei City (TW)

(72) Inventors: Ming-Tain LAI, Taipei City (TW); Wan-Fen LI, Taipei City (TW); Chun-Chung Wang, Taipei City (TW); Lu-Tzu Chen, Taipei City (TW)

(21) Appl. No.: 18/288,215

(22) PCT Filed: Apr. 28, 2021
(86) PCT No.: PCT/US2021/029552

§ 371 Date: Oct. 25, 2023

(51) Int. Cl. A61K 31/675; A61K 45/06; A61P 35/00; C07K 16/28

(52) U.S. Cl. CPC A61K 31/675; A61K 45/06; A61P 35/00; C07K 16/2818; C07K 16/2827

(57) ABSTRACT

A pharmaceutical composition, including a compound 1-(3-(3-N,N-dimethylaminocarbonyl)phenoxyl-4-mtrophenyl)-1-N ethyl-N,N′-bis(ethylene)phosphoramidate and at least one therapeutic agent including a chemotherapeutic agent or biological agent, and its medical use are provided.

### FIELD OF THE INVENTION

The present invention relates to a composition which includes a compound 1-(3-(3-N,N-dimethylaminocarbonyl)phenoxyl-4-mtrophenyl)-1-ethyl-N,N′-bis(ethylene)phosphoramidate combined with at least one therapeutic agent including a chemotherapeutic agent or biological agent and its medical use.

### BACKGROUND OF THE INVENTION

Cancer is one of the major causes of human morbidity and mortality. Cancer treatment is challenging because it is difficult to kill cancer cells without damaging or killing normal cells. Damaging or killing normal cells during cancer treatment is a cause of adverse side effects in patients and can limit the amount of chemotherapeutic agent administered to a cancer patient.

Aldo-keto reductase family 1 member C3 (AKR1C3) is an enzyme that encoded by the AKR1C3 gene in human. This gene encodes a member of the aldo/keto reductase superfamily, which consists of more than 40 known enzymes and proteins. These enzymes catalyze the conversion of aldehydes and ketones to their corresponding alcohols by utilizing NADH and/or NADPH as cofactors. It is also known as type 5, 17β-hydroxysteroid dehydrogenase (17β-HSD) and prostaglandin F synthase. AKR1C3 is one member of the 15 gene families of aldo-keto reductases (AKRs). AKR1C3 was originally cloned from human prostate⁽¹⁾ and placenta⁽²⁾ cDNA libraries. AKR1C3 is a monomeric, cytosolic, NAD(P) (H)-dependent oxidoreductase with 323 amino acids and a molecular weight of 37 kDa⁽¹⁾. AKR1C3 shares high sequence homology with the related human AKR1C family, including AKR1C1, AKR1C2, and AKR1C4. AKR1C3 catalyzes androgen, estrogen, progesterone, and prostaglandin (PG) metabolism and is subsequently involved in the regulation of nuclear receptor activities^{(3,4)}. AKR1C3 is expressed in normal tissues including steroid hormone-dependent and steroid hormone-independent cells with an average low expression level except in liver, kidney, and small intestine⁽⁵⁾. Many studies have demonstrated that AKR1C3 is abnormally overexpressed in many malignant solid and hematologic tumors. The data show that more than 50% of hepatoma, bladder, renal, and gastric cancers were detected with high expression of AKR1C3 with immunohistochemistry scores (IHC score) ≥4 on a scale of 0 to 6⁽⁶⁾. AKR1C3 is highly expressed in non-small cell lung cancer (NSCLC) but not in small cell-lung cancer⁽⁷⁾.

AKR1C3 upregulation in cancer is reported to be associated with metastasis of castrate-resistant prostate cancer (CRPC⁽⁸⁾) and colorectal cancer (CRC⁽⁹⁾), and is also linked to poor prognosis and a low survival rate^{(10,11)} In addition, many types of treatment resistance are attributed to the overexpression of AKR1C3. It has been reported that chemotherapy resistance to doxorubicin^{(12,13)} enzalutamide⁽¹⁴⁾ abiraterone⁽¹⁵⁾ and methotrexate⁽¹⁶⁾ is directly related to high AKR1C3 expression in cells. Radiotherapy resistance in esophageal cancer⁽¹⁷⁾, prostate cancer⁽¹⁸⁾ and NSCL cancer cells⁽¹⁹⁾ is associated with AKR1C3 overexpression. The main mechanism of action of AKR1C3 against ionizing radiation is to reduce ROS (reactive oxygen species) in cells, to increase PGF2α which subsequently leads to MAP kinase activation and PPARγ inhibition, resulting in a significant reduction in DNA damage⁽¹⁸⁾. Immunotherapy resistance is also attributed to AKR1C3 high expression. One study has shown that high expression of AKR1C3 is associated with the failure of PD-1-targeted therapies in PD-L1 positive patients with advanced renal cell carcinoma (RCC) based on whole genome microarray and multiplex quantitative (q)RT-PCR gene expression analysis⁽²⁰⁾. Due to tumor-specific overexpression of AKR1C3, the design of AKR1C3-activated prodrugs becomes an attractive approach to specifically target cancer. One such example is the AKR1C3-activated prodrug, PR104, which exhibited good anti-tumor activity in vitro and in vivo^{(6,21)} although it was originally designed as a hypoxia-activated prodrug⁽²²⁻²⁴⁾.

Anti-cancer prodrug of Formula I-1 of the present application (denoted by OBI-3424 herein) is a chemically synthesized potent nitrogen mustard, which is selectively cleaved to the cytotoxic aziridine (denoted by OBI-2660 herein) by AKR1C3 in the presence of NADPH. The active molecule OBI-2660 released by OBI-3424 is similar to the standard chemotherapeutic drugs thiotepa and mitomycin C, which leads to alkylation and cross-linking of DNA at the N7 (or O6) position of guanine. Prodrug OBI-3424 is currently under development by Ascentawits Pharmaceuticals, LTD in Asian countries and by OBI Pharma, Inc. in countries outside Asia (drug code OBI-OBI-3424) for the treatment of malignant tumors. Prodrug OBI-3424 is currently being investigated in multiple Phase I clinical trials in the US (NCT04315324 & NCT03592264) and in China (CXHL1900137 & CXHL2000263) to treat more than 14 types of human cancer, including solid tumors and hematologic malignancies. Due to the high expression of AKR1C3 in tumors, prodrug OBI-3424 is designed to be specifically activated in tumors but spared in normal cells which express low levels of AKR1C3 to achieve tumor-specific targeting. Furthermore, tumor-selective activation of OBI-3424 is distinguishable from non-selective traditional alkylating agents, such as cyclophosphamide and ifosfamide, indicating that OBI-3424 has the potential to become a broad-spectrum, highly selective anti-tumor drug. Prodrug OBI-3424 was reported to exhibit potent efficacy against preclinical models of T-ALL in vitro and in vivo^{(25,26)}.

In the presence of NADPH, reduction of OBI-3424 is mediated by AKR1C3 to release the cytotoxic moiety OBI-2660, which is an aziridine bis-alkylating agent, leading to cross-linking of DNA at the N7 (or O6) position of guanine, and subsequent cell death.

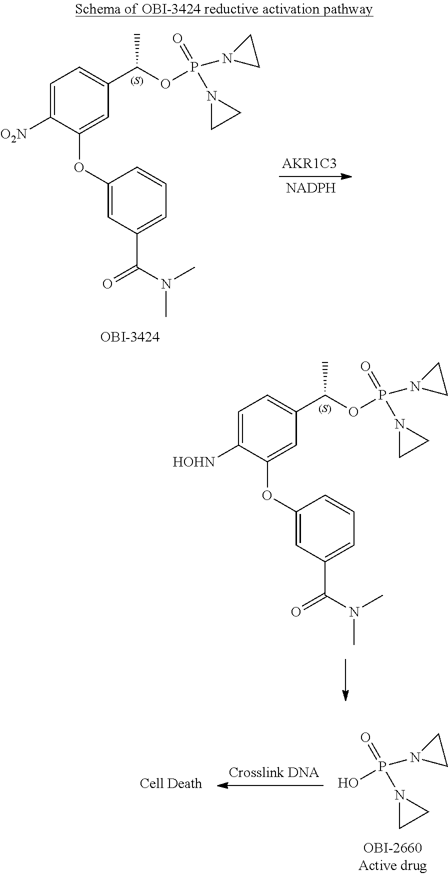

Prodrugs designed to target cancer cells have emerged as an attractive strategy for cancer therapy in recent years; however, many prodrugs failed in Phase 3 clinical trials due to a lack of valid biomarkers to select patients⁽²⁷⁾. Given that the AKR1C3 expression can be assessed using RT-PCR or immunohistochemistry, OBI-3424 can be developed in a clinically efficient manner by selecting patients who have high AKR1C3 expression and are most likely to respond to the prodrug. AKR1C3 has been demonstrated to be overexpressed upon acquisition of chemoresistance^{(13,14)} radioresistance⁽¹⁹⁾ and immunoresistance⁽²⁰⁾. In addition, cancers with homologous recombination deficiency (HRD) such as ovarian, breast, and pancreatic cancers, are known to be sensitive to DNA damaging agents⁽²⁸⁾. As a DNA alkylator, OBI-3424 may also be a good candidate drug to treat HRD cancers that have AKR1C3 expression.

There remains a need for a compound suitable for treating cancer patients, which is a selective AKR1C3 reductase activated prodrug, and a novel, selective and broad anti-cancer agent. The present invention meets this need.

Program death 1 (PD-1) is an inhibitory receptor expressed on T cells, B cells, or monocytes^{(29, 30)} PD-L1 and PD-L2 are ligands for PD-1 which have been identified to downregulate T cell activation and cytokine secretion upon binding to PD-1^{(31, 32)} Engagement of PD-1 with PD-L1 or PD-L2 leads to down-regulation of immune responses. Hence, blocking of the PD-1/PD-L1 pathway has been proposed to attenuate central and peripheral immune responses against cancer. Targeting PD1 and PD-L1 pathway have shown the clinical efficacy in more than 15 cancer types including melanoma, non-small cell lung cancer (NSCLC), renal cell carcinoma (RCC), bladder carcinoma and Hodgkin's lymphoma⁽³³⁾. However, there are still many patients fail to respond; some patients showed initial responses but acquire resistance overtime. Therefore, there is an urgent need to identify mechanisms of resistance for combination therapy.

### SUMMARY OF THE INVENTION

The present invention, based on the compounds or pharmaceutically acceptable salts, or solvates thereof as disclosed in PCT Patent Application No. PCT/US2016/062114 (WO2017087428A1), provides medical use of the compounds, and provides compositions including the compounds or pharmaceutically acceptable salts, isotopic variants or solvates thereof and their anti-cancer medical use.

In one aspect, the present invention provides use of the compound 1-(3-(3-N,N-dimethylaminocarbonyl)phenoxyl-4-mtrophenyl)-1-ethyl-N,N′-bis(ethylene)phosphoramidate represented by Formula I (denoted by OBI-2870 herein), or a pharmaceutically acceptable salt, isotopic variant or solvate thereof in the manufacture of a medicament for treating cancer in a patient, wherein the AKR1C3 reductase level of the cancer is represented by the AKR1C3 protein level or RNA level and is equal to or greater than a predetermined value. AKR1C3 levels are measured following routine methods well known to the skilled artisan.

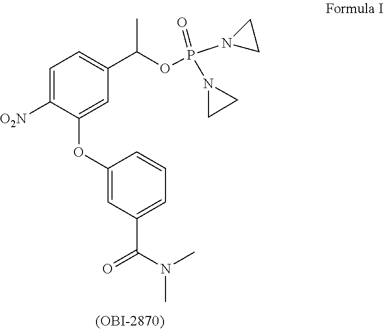 [SMILES: CC(OP(=O)(N1CC1)N1CC1)c1ccc([N+](=O)[O-])c(Oc2cccc(C(=O)N(C)C)c2)c1]

According to particular embodiments of the invention, the compound is (S)-1-(3-(3-N,N-dimethylaminocarbonyl)phenoxyl-4-mtrophenyl)-1-ethyl-N,N′-bis(ethylene)phosphoramidate represented by Formula I-1 (denoted by OBI-OBI-3424 herein), or (R)-1-(3-(3-N,N-dimethylaminocarbonyl)phenoxyl-4-mtrophenyl)-1-ethyl-N,N′-bis(ethylene)phosphoramidate represented by Formula I-2 (denoted by OBI-3423 herein).

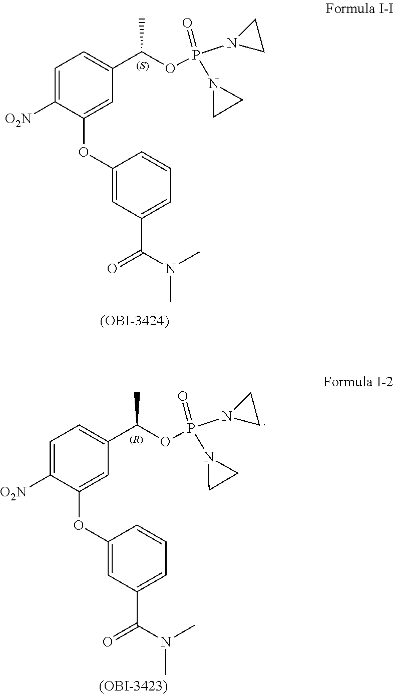

The preparation of the compound of Formula I, Formula I-1 or Formula I-2 is disclosed in PCT Patent Application No. PCT/US2016/062114 (WO2017087428A1), the disclosures of which are incorporated herein by reference in its entirety. Herein, compound OBI-2870 is a racemic mixture of R-enantiomer 3423 and S-enantiomer OBI-3424 at 1:1 ratio.

Herein, the salts may be basic salts, including the salts of the compounds with an inorganic base (such as alkali metal hydroxide and alkaline earth metal hydroxide) or with an organic base (such as monoethanolamine, diethanolamine or triethanolamine). Alternatively, the salts may be acid salts, including the salts of the compounds with an inorganic acid (such as hydrochloric acid, hydrobromic acid, hydroiodic acid, nitric acid, perchloric acid, sulfuric acid or phosphoric acid) or with an organic acid (such as methanesulfonic acid, trifluoromethanesulfonic acid, ethanesulfonic acid, benzenesulfonic acid, p-toluenesulfonic acid, fumaric acid, oxalic acid, maleic acid and citric acid). It is a well-known technology in the art to select and prepare acceptable salts, solvates, and the like of a compound.

According to particular embodiments of the invention, the compound of Formula I-1 or Formula I-2 has an enantiomeric excess of no less than 80%. Preferably, the compound has an enantiomeric excess of no less than 90%, more preferably, no less than 95%.

According to particular embodiments of the invention, the compound of Formula I-1 or Formula I-2 is substantially pure.

According to particular embodiments of the invention, the cancer is liver cancer, hepatocellular carcinoma (HCC), lung cancer, melanoma, prostate cancer, breast cancer, leukemia, esophageal cancer, renal cancer, gastric cancer, colon cancer, brain cancer, bladder cancer, cervical cancer, ovarian cancer, head and neck cancer, endometrial cancer, pancreatic cancer, a sarcoma cancer, or rectal cancer.

According to particular embodiments of the invention, the cancer is liver cancer.

The dosage of the medicament used for treating cancer, or the dosage of the compound or salt, isotopic variant or solvate thereof, or the other chemotherapeutic agent contained in the medicament usually depends on the specific compound applied, the patient, the specific disease or condition and the severity thereof, the route and frequency of administration and the like, and needs to be determined by the attending physician according to specific conditions. For the purpose of the present disclosure, a typical daily dosage might range from about any of 0.1 μg/kg to 1 μg/kg, to 10 μg/kg, to 100 μg/kg, to 1 mg/kg, to 10 mg/kg, to 100 mg/kg, or more, depending on the factors mentioned above. For repeated administrations over several days or longer, depending on the condition, the treatment is sustained until a desired suppression of symptoms occurs or until sufficient therapeutic levels are achieved to alleviate cancer, or a symptom thereof. An exemplary dosing regimen includes administering an initial dose of about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg or more followed by a weekly maintenance dose. However, other dosage regimens may be useful, depending on the pattern of pharmacokinetic decay that the practitioner wishes to achieve. For example, dosing from one-four times a week is contemplated. In certain embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays.

The medicament can be any dosage form for clinical administration, such as tablets, suppositories, dispersible tablets, enteric-coated tablets, chewable tablets, orally disintegrating tablets, capsules, sugar coated agents, granules, dry powders, oral solutions, a small needle for injection, lyophilized powder for injection, or infusion solutions.

According to particular embodiments of the invention, the method further includes a step for measuring the content of AKR1C3 reductase of cancer cells in a patient using AKR1C3 antibodies, where the content of AKR1C3 reductase is measured to be equal to or greater than the predetermined value, and the compound is administered to the patient.

In another aspect, the invention provides a method for inhibiting the growth of a cell, including the step of contacting the cell with an effective amount of compound 1-(3-(3-N,N-dimethylaminocarbonyl)phenoxyl-4-mtrophenyl)-1-ethyl-N,N′-bis(ethylene)phosphoramidate represented by Formula I, or a pharmaceutically acceptable salt, isotopic variant or solvate thereof; wherein the AKR1C3 reductase level of the cell is represented by the AKR1C3 protein level or RNA level and is equal to or greater than a predetermined value.

According to particular embodiments of the invention, the method further includes a step for measuring the content of AKR1C3 reductase of cell using AKR1C3 antibodies, where the content of AKR1C3 reductase is measured to be equal to or greater than the predetermined value, and the compound is contacted with the cell.

In another aspect, the invention provides use of the compound 1-(3-(3-N,N-dimethylaminocarbonyl)phenoxyl-4-mtrophenyl)-1-ethyl-N,N′-bis(ethylene)phosphoramidate represented by Formula I, or a pharmaceutically acceptable salt, isotopic variant or solvate thereof in the manufacture of a medicament for inhibiting the growth of a cell; wherein the AKR1C3 reductase level of the cell is represented by the AKR1C3 protein level or RNA level and is equal to or greater than a predetermined value.

According to particular embodiments of the invention, the cell is a cancer cell.

In another aspect, the invention provides a composition including:
(1) the compound 1-(3-(3-N,N-dimethylaminocarbonyl)phenoxyl-4-mtrophenyl)-1-ethyl-N,N′-bis(ethylene)phosphoramidate represented by Formula I, or a pharmaceutically acceptable salt, isotopic variant or solvate thereof, and
(2) at least one therapeutic agent including a chemotherapeutic agent or biological agent.

According to particular embodiments of the invention, the compound is (S)-1-(3-(3-N,N-dimethylaminocarbonyl)phenoxyl-4-mtrophenyl)-1-ethyl-N,N′-bis(ethylene)phosphoramidate represented by Formula I-1, or (R)-1-(3-(3-N,N-dimethylaminocarbonyl)phenoxyl-4-mtrophenyl)-1-ethyl-N,N′-bis(ethylene)phosphoramidate represented by Formula I-2.

According to particular embodiments of the invention, the anti-PD-1/PD-L1 antibody is Bavencio® (avelumab), Opdivo® (nivolumab), Keytruda® (pembrolizumab), Imfinzi® (durvalumab) and/or Tecentriq® (atezolizumab).

According to particular embodiments of the invention, in the case where the cancer is liver cancer, the anti-PD-1 antibody is Keytruda (pembrolizumab) and the anti-PD-1 antibody is Bavencio® (avelumab).

According to particular embodiments of the invention, the composition further includes a pharmaceutically acceptable excipient. Preferably, the excipient is selected from inert diluents, dispersing and/or granulating agents, surface active agents and/or emulsifiers, disintegrating agents, binding agents, preservatives, buffering agents, lubricating agents and oils.

In another aspect, the invention provides a method for treating cancer in a patient in need thereof, including the step of administering to the patient an effective amount of the composition according to the invention.

According to particular embodiments of the invention, the method further includes a step for measuring the content of AKR1C3 reductase of cancer cells in a patient using AKR1C3 antibodies, where the content of AKR1C3 reductase is measured to be equal to or greater than the predetermined value, the composition is administered to the patient.

Therefore, the embodiment of this present invention relates to a combination including a compound of Formula I (OBI-2870), Formula I-1 (OBI-3424) or Formula I-2 (OBI-3423) and at least one inhibitor of the inhibitory immune checkpoint antigen. In certain specific embodiment, the immune checkpoint inhibitor is an anti-immune checkpoint antibody which inhibit/block the inhibitory immune checkpoint antigen.

In one embodiment, the inhibitory immune checkpoint antigen is selected from the group consisting of PD-1/PD-L1 antigen, CTLA-4 (Cytotoxic T-lymphocyte-Associated Protein 4), LAG-3 (Lymphocyte Activation Gene 3), TIGIT (T-cell ImmunoGlobulin and Immunoreceptor Tyrosine-based inhibitory motif domain), Ceacam 1 (Carcinoembryonic antigen-related cell adhesion molecule 1), LAIR-1 (leucocyte-associated immunoglobulin-like receptor-1), TIM-3 (T cell Immunoglobulin and Mucin domain-3), VISTA (V-domain Ig suppressor of T cell activation), KIR (Killer-cell Immunoglobulin-like Receptor), IDO (Indoleamine-pyrrole 2,3-dioxygenase), B7-H3 (CD276), A2AR (Adenosine A2A receptor) or CD47.

In one embodiment, the anti-immune checkpoint antibody is an anti-PD-1/PD-L1 antibody, an anti-CTLA-4 (Cytotoxic T-lymphocyte-Associated Protein 4) antibody, an anti-LAG-3 (Lymphocyte Activation Gene 3) antibody, an anti-TIGIT (T-cell ImmunoGlobulin and Immunoreceptor Tyrosine-based inhibitory motif domain) antibody, an anti-Ceacam 1 (Carcinoembryonic antigen-related cell adhesion molecule 1) antibody, an anti-LAIR-1 (leucocyte-associated immunoglobulin-like receptor-1) antibody, an anti-TIM-3 (T cell Immunoglobulin and Mucin domain-3) antibody, an anti-VISTA (V-domain Ig suppressor of T cell activation) antibody, an anti-KIR (Killer-cell Immunoglobulin-like Receptor) antibody, an anti-IDO (Indoleamine-pyrrole 2,3-dioxygenase) antibody, an anti-B7-H3 (anti-CD276) antibody, an anti-A2AR (Adenosine A2A receptor) antibody or an anti-CD47 antibody.

In one embodiment, the anti-PD-1/PD-L1 antibody is Bavencio® (avelumab), Opdivo® (nivolumab), Keytruda® (pembrolizumab), Imfinzi® (durvalumab) and/or Tecentriq® (atezolizumab).

### BRIEF DESCRIPTION OF DRAWINGS

FIG. 1. Mean body weight in each group of Example 1. The body weight of vehicle, single treatment and combination treatment groups were recorded twice weekly until Day 35. Body weight loss was not seen in all the treatment groups in human hepatocellular carcinoma HepG2 tumor bearing humanized mice. Data was shown as the mean±SEM (N=5 for each group). Statistical analyses were performed by Student's t-test.

FIG. 2. Mean tumor weight in each group of Example 1. Mice were sacrificed on Day 36 after tumor cell inoculation and the tumor weight of mice in vehicle, single treatment and combination treatment groups were recorded. Tumor weight in the OBI-3424 (G2) and OBI-3424+anti-hPD-1/anti-hPD-L1(G5/G6) combined treatment groups were significantly suppressed compared with that in the vehicle (G1) group (p<0.001). Moreover, tumor weight in the OBI-3424+anti-hPD-1/anti-hPD-L1(G5/G6) combined treatment groups were significantly suppressed compared with that in the anti-hPD-1/anti-hPD-L1(G3/G4) treatment groups (p<0.05). Data was shown as the mean±SEM (N=5 for each group). Statistical analyses were performed by Student's t-test. P values <0.05 were considered significant. Single stars denote 0.05<P<0.001, double stars P<0.001.

FIG. 3. Mean tumor volume in each group of Example 1. The tumor volume of mice in vehicle, single treatment and combination treatment groups were recorded twice weekly until Day 35. Tumor volume in the OBI-3424 (G2) and OBI-3424+anti-hPD-1/anti-hPD-L1(G5/G6) combined treatment groups were significantly suppressed compared with that in the vehicle (G1) group. Data was shown as the mean±SEM (N=5 for each group). Statistical analyses were performed by Student's t-test.

FIG. 4. Mean body weight in each group of Example 2. The body weight of vehicle, OBI-3424 low-dose and high-dose treatment, and OBI-3424 plus PD-1 antibody combined treatment groups were recorded twice weekly until Day 30. Body weight loss was not seen in the vehicle, OBI-3424 single or OBI-3424 plus PD-1 antibody combination treatment groups in human hepatocellular carcinoma HepG2 tumor bearing humanized mice. Data was shown as the mean±SEM (N=5 for each group). Statistical analyses were performed by Student's t-test.

FIG. 5. Mean tumor weight in each group of Example 2. Mice were sacrificed on Day 30 after test item administration and the tumor weight of mice in OBI-3424 low-dose (G2), high-dose treatment (G3) and OBI-3424 plus PD-1 antibody combined treatment (G5-G7) were significantly suppressed compared with that in the vehicle (G1) group (p<0.05). Moreover, tumor weight in the high-dose OBI-3424 plus PD-1 antibody combined treatment (G6) was significantly reduced compared with that in high-dose OBI-3424 single treatment (G3) (p<0.05). Data was shown as the mean±SEM. Statistical analyses were performed by Student's t-test. P values <0.05 were considered significant. Single stars denote 0.05<P<0.001, double stars P<0.001.

FIG. 6. Mean tumor volume in each group of Example 2. The tumor volume of mice in vehicle, OBI-3424 low-dose and high-dose treatment, and OBI-3424 plus PD-1 antibody combined treatment groups were recorded twice weekly until Day 30. Tumor volume in the OBI-3424 high-dose treatment (G3) and OBI-3424 plus anti-hPD-1 (G5-G7) combined treatment groups were significantly suppressed compared with that in the vehicle (G1) group. Data was shown as the mean±SEM (N=5 for each group). Statistical analyses were performed by Student's t-test.

### DETAILED DESCRIPTION OF THE INVENTION

The present invention will be described below with reference to specific examples. Those skilled in the art could understand that these examples are only used for describing the invention and do not in any way limit its scope.

### Definition

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

All numerical designations, e.g., pH, temperature, time, concentration, and weight, including ranges of each thereof, are approximations that typically may be varied (+) or (−) by increments of 0.1, 1.0, or 10.0, as appropriate. All numerical designations may be understood as preceded by the term “about”. Reagents described herein are exemplary and equivalents of such may be known in the art.

The term “A”, “an” and “the” include plural referents unless the context clearly dictates otherwise. Thus, for example, reference to a compound refers to one or more compounds or at least one compound. As such, the terms “a” (or “an”), “one or more”, and “at least one” are used interchangeably herein.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3% 2%, 1%, 0.5%, or 0.05% of a given value or range.

As used herein, the term “comprising” or “including” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising/including) or alternatively including steps and compositions of no significance (consisting essentially of) or alternatively, intending only the stated method steps or compositions (consisting of).

The term “Administering” or “administration of” a drug to a patient (and grammatical equivalents of this phrase) refers to direct administration, which may be administration to a patient by a medical professional or may be self-administration, and/or indirect administration, which may be the act of prescribing a drug. For example, a physician who instructs a patient to self-administer a drug and/or provides a patient with a prescription for a drug is administering the drug to the patient.

The term “antibody” is further intended to encompass antibodies, digestion fragments, specified portions and variants thereof, including antibody mimetics or including portions of antibodies that mimic the structure and/or function of an anti-cancer antibody or specified fragment or portion thereof, including single chain antibodies and fragments thereof, each containing at least one CDR derived from an anti-cancer antibody of the present invention.

The term “biological agent” includes peptides, proteins, antibodies, hormones, cytokines, chemokines, and any combination thereof.

The term “Cancer” refers to leukemias, lymphomas, carcinomas, and other malignant tumors, including solid tumors, of potentially unlimited growth that can expand locally by invasion and systemically by metastasis. Examples of cancers include, but are not limited to, cancer of the adrenal gland, bone, brain, breast, bronchi, colon and/or rectum, gallbladder, head and neck, kidney, larynx, liver, lung, neural tissue, pancreas, prostate, parathyroid, skin, stomach, and thyroid. Certain other examples of cancers include, acute and chronic lymphocytic and granulocytic tumors, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myeloma, mycosis fungoides, neuroblastoma, osteo sarcoma, osteogenic and other sarcoma, ovarian tumor, pheochromocytoma, polycythermia vera, primary brain tumor, small-cell lung tumor, squamous cell carcinoma of both ulcerating and papillary type, hyperplasia, seminoma, soft tissue sarcoma, retinoblastoma, rhabdomyosarcoma, renal cell tumor, topical skin lesion, veticulum cell sarcoma, and Wilm's tumor.

The term “chemotherapeutic agent”, as used herein, is a chemical compound useful in the treatment of cancer. Examples of chemotherapeutic agents include Monomethyl auristatin E (MMAE), Monomethyl auristatin F (MMAF), mertansine (DM1), anthracycline, pyrrolobenzodiazepine, α-amanitin, tubulysin, benzodiazepine, erlotinib, bortezomib, fulvestrant, sunitinib, letrozole, imatinib mesylate, PTK787/ZK 222584, oxaliplatin, leucovorin, rapamycin, lapatinib, lonafarnib (SARASAR®, SCH 66336), sorafenib, gefitinib, AG1478, AG1571, alkylating agent, alkyl sulfonate, aziridines, ethylenimine, methylamelamine, acetogenins, camptothecin, bryostatin, callystatin, CC-1065, cryptophycins, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, calicheamicin, dynemicin, clodronate, esperamicin, neocarzinostatin chromophore, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, epirubicin, esorubicin, idarubicin, marcellomycin, mitomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, epothilone, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, razoxane, rhizoxin, sizofiran, spirogermanium, tenuazonic acid, triaziquone, 2,2′,2″-trichlorotriethylamine, trichothecene, urethan, vindesine, dacarbazine, mannomustine, mitobronitol, mitolactol, pipobroman, gacytosine, arabinoside, cyclophosphamide, thiotepa, taxoid, paclitaxel, doxetaxel, chloranbucil, gemcitabine, 6-thioguanine, mercaptopurine, methotrexate, cisplatin, carboplatin, vinblastine, platinum, etoposide, ifosfamide, mitoxantrone, vincristine, vinorelbine, novantrone, teniposide, edatrexate, daunomycin, aminopterin, xeloda, ibandronate, topoisomerase inhibitor, difluoromethylornithine (DMFO), retinoid and capecitabine.

The term “combination” refers to combination therapy would be the amount of the OBI-3423/OBI-3424 compound and/or the amount of other biological or chemical drugs that when administered together (either as co-administration and/or co-formulation), either sequentially or simultaneously, on the same or different days during a treatment cycle, have a synergistic effect that is therapeutically effective and more than therapeutically additive.

The term “contacting” or “contact” is meant to refer to bringing together of a therapeutic agent and cell or tissue such that a physiological and/or chemical effect takes place as a result of such contact. Contacting can take place in vitro, ex vivo, or in vivo. In one embodiment, a therapeutic agent is contacted with a cell in cell culture (in vitro) to determine the effect of the therapeutic agent on the cell. In another embodiment, the contacting of a therapeutic agent with a cell or tissue includes the administration of a therapeutic agent to a subject having the cell or tissue to be contacted.

The terms “optically active” refers to a collection of molecules, which has an enantiomeric excess of no less than about 10%, no less than about 20%, no less than about 30%, no less than about 40%, no less than about 50%, no less than about 60%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, no less than about 99.8%, or no less than about 99.9%. In certain embodiments, the enantiomeric excess for an optically active compound is no less than about 90%, no less than about 95%, no less than about 98%, or no less than about 99%. An enantiomeric excess of a compound can be determined by any standard methods used by one of ordinary skill in the art, including, but not limited to, chiroptical chromatography (gas chromatography, high-performance liquid chromatography, and thin-layer chromatography) using an optically active stationary phase, isotopic dilution, electrophoresis, calorimetry, polarimetry, NMR resolution methods with chiral derivatization, and NMR methods with a chiral solvating agent or chiral shift reagent.

In describing an optically active compound, the prefixes R and S are used to denote the absolute configuration of the molecule about its chiral center(s).

The terms “substantially pure” means sufficiently homogeneous to appear free of readily detectable impurities as determined by standard analytical methods used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” refers to a collection of molecules, wherein at least about 50%, at least about 70%, at least about 80%, at least about 90%, at least about 95%, at least about 98%, at least about 99%, or at least about 99.5% by weight of the molecules are a single stereoisomer of a compound, as determined by standard analytical methods.

“Patient” and “subject” are used interchangeably to refer to a mammal in need of treatment for cancer. Generally, the patient is a human. Generally, the patient is a human diagnosed with cancer. In certain embodiments, a “patient” or “subject” may refer to a non-human mammal used in screening, characterizing, and evaluating drugs and therapies, such as, a non-human primate, a dog, cat, rabbit, pig, mouse or a rat.

The term “Prodrug” refers to a compound that, after administration, is metabolized or otherwise converted to a biologically active or more active compound (or drug) with respect to at least one property. A prodrug, relative to the drug, is modified chemically in a manner that renders it, relative to the drug, less active or inactive, but the chemical modification is such that the corresponding drug is generated by metabolic or other biological processes after the prodrug is administered. A prodrug may have, relative to the active drug, altered metabolic stability or transport characteristics, fewer side effects or lower toxicity, or improved flavor (for example, see the reference Nogrady, 1985, Medicinal Chemistry A Biochemical Approach, Oxford University Press, New York, pages 388-392, incorporated herein by reference). A prodrug may be synthesized using reactants other than the corresponding drug.

The term “Solid tumor” refers to solid tumors including, but not limited to, metastatic tumors in bone, brain, liver, lungs, lymph node, pancreas, prostate, skin and soft tissue (sarcoma).

The term “Therapeutically effective amount” of a drug refers to an amount of a drug that, when administered to a patient with cancer, will have the intended therapeutic effect, e.g., alleviation, amelioration, palliation or elimination of one or more manifestations of cancer in the patient. A therapeutic effect does not necessarily occur by administration of one dose, and may occur only after administration of a series of doses. Thus, a therapeutically effective amount may be administered in one or more administrations.

The term “Treatment of” a condition or patient refers to taking steps to obtain beneficial or desired results, including clinical results. For purposes of this invention, beneficial or desired clinical results include, but are not limited to, alleviation or improvement of one or more symptoms of cancer; diminishment of extent of disease; delay or slowing of disease progression; alleviation, palliation, or stabilization of the disease state; or other beneficial results. Treatment of cancer may, in some cases, result in partial response or stable disease.

The term “Tumor cells” refers to tumor cells of any appropriate species, e.g., mammalian such as murine, canine, feline, equine or human.

The term “isotopic variant” refers to a compound that contains an unnatural proportion of an isotope at one or more of the atoms that constitute such compounds. In certain embodiments, an “isotopic variant” of a compound contains unnatural proportions of one or more isotopes, including, but not limited to, hydrogen (¹H), deuterium (²H), tritium (³H), carbon-11 (¹¹C) carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I) In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (2H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a compound is in an unstable form, that is, radioactive. In certain embodiments, an “isotopic variant” of a compound contains unnatural proportions of one or more isotopes, including, but not limited to, tritium (3H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, for example, or any carbon can be ¹³C, as example, or any nitrogen can be ¹⁵N, as example, and any oxygen can be ¹⁸O, where feasible according to the judgment of one of skill. In certain embodiments, an “isotopic variant” of a compound contains unnatural proportions of deuterium.

The term “solvate” refers to a complex or aggregate formed by one or more molecules of a solute, e.g., a compound provided herein, and one or more molecules of a solvent, which is present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The term “pharmaceutically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response.

The term “immune checkpoint inhibitors” which are molecules that inhibit/block the inhibitory immune checkpoint system have emerged as effective therapies for advanced neoplasia; among these are therapeutic antibodies that block cytotoxic T lymphocyte associated antigen 4 (CTLA4) and programmed cell death protein 1 (PD-1), that have been used for several tumors⁽³⁴⁾. PD-1 (Programmed cell Death protein, CD279), (a member of the B7/CD28 family of receptors, is a monomeric molecule expressed on the cell surface of activated leucocytes, including T, B, NK and myeloid-derived suppressor cells, whose expression is finely regulated by an interplay between genetic and epigenetic mechanisms. Known ligands of PD-1 are PD-L1 and PD-L2⁽³⁵⁾.

PD-L1 (Programmed cell Death Protein Ligand 1, B7H1, CD274) is expressed at low levels, and up-regulated upon cell activation, on hematopoietic cells, including T, B, myeloid, and dendritic cells, and non-hematopoietic (such as lung, heart, endothelial, pancreatic islet cells, keratinocytes) and specially cancer cells. PD-L2 (Programmed cell Death Protein Ligand 2, B7-DC, CD273) is expressed on macrophages, dendritic cells (DCs), activated CD4⁺ and CD8⁺ lymphocytes and some solid tumors (ovarian carcinoma, small cell lung cancer, esophageal cancer). PD-L1 and PD-L2 expression has also been detected on normal and cancer-associated fibroblasts Both PD-L1 and PD-L2 interact with additional receptors: PD-L1 with the CD28 ligand CD80 and PD-L2 with Repulsive Guidance Molecule (RGM) b, expressed on macrophages and other cell types. The cytoplasmic tail of PD-1 contains an Immunoreceptor Tyrosine-based Inhibition Motif (ITIM) and an immunoreceptor tyrosine-based switch motif (ITSM). In T lymphocytes, PD-1 interaction with its ligands results in the phosphorylation of two tyrosines at the intracellular tail of PD-1; the recruitment of SH2 domain-containing protein tyrosine phosphatases (SUP-1 and/or SHP-2) to the ITSM cytoplasmic region of PD-1 then inhibits downstream signals of the T-cell receptor, thereby inhibiting T cell proliferation and cytokine production. PD-1 exerts also other effects on T cells: for example, by inhibiting Akt and Ras pathways, PD-1 triggering suppresses transcription of the ubiquitin ligase component SKP2: this results in impairing SKP2-mediated degradation of p27 (kip1), an inhibitor of cyclin-dependent kinases, and thereby in blocking cell cycle progression. In addition, PD-1 can promote apoptosis by more than one mechanism Besides directly inhibiting T cell activation, PD-1 triggering by PD-L1 can induce the development of T regulatory cells (Treg), key mediators of peripheral tolerance that actively suppress effector T cells. Treg induction by PD-1 triggering is mediated by modulation of key signaling molecules, such as phospho-Akt, whose levels are kept low by the PD-1-induced activity of PTEN. Several types of cancer cells do express PD-L1. Furthermore, non-neoplastic cells (endothelial cells, leucocytes, fibroblasts) in the tumor microenvironment can also express PD-L1. This suggests that they can tolerate tumor-infiltrating PD-1⁺ T lymphocytes (TILs), and/or induce Treg development; indeed a growing body of evidence indicate that treatment of patients affected by some cancer types (melanoma, renal carcinoma, Non-Small Cell Lung Cancer, etc.) with anti-PD-1/PD-L1 monoclonal antibodies (mAbs) can reduce tumor growth.

Immune checkpoint inhibitors are known to provide some anti-tumor activity in humans, and this partial anti-tumor activity is only observed in a fraction of treated subjects. Checkpoint inhibitors can include or exclude proteins, polypeptides, including amino acid residues and monoclonal or polyclonal antibodies. The compositions described herein can include or be administered along with more than one checkpoint inhibitor. In some embodiments, the checkpoint inhibitors bind to ligands or proteins that are found on any of the family of T cell regulators, including CD28/CTLA-4. Targets of checkpoint inhibitors can include or exclude receptors or co-receptors (e.g., CTLA-4; CD8) expressed on immune system effector or regulator cells (e.g., T cells); proteins expressed on the surface of antigen-presenting cells (i.e., expressed on the surface of activated T cells, which can include or exclude PD-1, PD-2, PD-L1 and PD-L2); metabolic enzymes or metabolic enzymes that are expressed by both tumor and tumor-infiltrating cells (e.g., Indoleamine-pyrrole 2,3-dioxygenase (IDO), including isoforms, such as IDO1 and IDO2); proteins that belong to the immunoglobulin superfamily (e.g., lymphocyte-activation gene 3, also known as LAG3); proteins that belong to the B7 superfamily (e.g., B7-H3/CD276 or homologs thereof). B7 proteins can be found on both activated antigen presenting cells and T cells. In some embodiments, two or more checkpoint inhibitors can be combined or paired together. For example, a B7 family checkpoint inhibitor, found on an antigen presenting cell, can be paired with a CD28 or CTLA-4 inhibitor, expressed on surface of a T cell, to produce a co-inhibitory signal to decrease the activity between these two types of cells. A co-receptor refers to the presence of two different receptors located on the same cell that after binding to an external ligand can regulate internal cellular processes. Co-receptors can be stimulatory or inhibitory. Co-receptors are sometimes called accessory receptors or co-signally receptors. As used herein, the term “co-inhibitory,” refers to the result of more than one molecule binding to their respective receptors on the surface of a cell thereby slowing down or preventing an intracellular process from occurring.

In certain embodiments, immune checkpoint inhibitors can include an antagonist of an inhibitory receptor which inhibits the PD-1 or CTLA-4 pathway, such as an anti-PD-1, anti-PD-L1 or anti-CTLA-4 antibody or inhibitor. Examples of PD-1 or PD-L1 inhibitors can include, without limitation, humanized antibodies blocking human PD-1 such as lambrolizumab (anti-PD-1 Ab, trade name Keytruda®) or pidilizumab (anti-PD-1 Ab), Bavencio® (anti-PD-L1 Ab, avelumab), Imfinzi® (anti-PD-L1 Ab, durvalumab), and Tecentriq® (anti-PD-L1 Ab, atezolizumab) as well as fully human antibodies such as nivolumab (anti-PD-1 Ab, trade name Opdivo®). Other PD-1 inhibitors may include presentations of soluble PD-1 ligand including without limitation PD-L2 Fc fusion protein also known as B7-DC-Ig or AMP-244 and other PD-1 inhibitors presently under investigation and/or development for use in therapy. In addition, immune checkpoint inhibitors may include without limitation humanized or fully human antibodies blocking PD-L1 such as durvalumab and MIH1 and other PD-L1 inhibitors presently under investigation. In some embodiments, the immune checkpoint inhibitor is CTLA-4, PD-L1 or PD-1 antibodies. In some embodiments, the PD-1 or CTLA-4 inhibitors include without limitation humanized antibodies blocking human PD-1 such as lambrolizumab (anti-PD-1 Ab, trade name Keytruda) or pidilizumab (anti-PD-1 Ab), nivolumab (anti-PD-1 Ab, trade name Opdivo), ticilimumab (anti-CTLA-4 Ab), ipilimumab (anti-CTLA-4 Ab), MPDL3280A, BMS-936559, AMP-224, EMIP321 (ImmuFact), MGA271, Indoximod, and INCB024360.

### EXAMPLES

### Example 1. Efficacy Evaluation of OBI-3424+Anti-PD-1 (Pembrolizumab) or Anti-PD-L1 Antibody (Avelumab) in HepG2 Tumor Bearing Humanized Mice Model

The aim of study is to evaluate the efficacy of test item OBI-3424 single therapy or combined treatments in the presence of anti-hPD-1 antibody or anti-hPD-L1 antibody in HepG2 tumor bearing humanized mouse model.

### Material

1. OBI-3424-DP
Lot number: FLC-INJ-1711-01
Number of test item: 2 vials/1 mL per vial
Ingredient: DNA alkylating agents
Concentration: 10 mg/mL
Physical appearance: clear liquid
Storage condition: −20° C.

2. Anti-Human PD-1, Pembrolizumab, Merck & Co., Inc.
Lot number: 7302614A13
Number of test item: 1 vials/1.2 mL per vial
Ingredient: antibody
Concentration: 25 mg/mL
Physical appearance: clear liquid
Storage condition: 2-8° C.

3. Anti-Human PD-L1, Avelumab, Merck & Co., Inc.
Lot number: AU024788
Number of test item: 1 tube/1.5 mL per tube
Ingredient: antibody
Concentration: 20 mg/mL
Physical appearance: clear liquid
Storage condition: 2-8° C.

4. Sterile Saline (Taiwan Biotech Co., LTD)
Lot number: 1PD2A054
Number of test item: 6 tubes of 20 mL tube
Concentration: 0.9% Sodium chloride
Physical appearance: clear liquid
Solubility: not provided
Storage condition: Room Temperature

5. Matrigel (Corning, Cat. No.: 354248, Lot No.: 8228001)

6. Human PBMC (Lot: PBMC102219D, Zenbio, USA)

7. Collagenase (Sigma Aldrich, C5138), DNase I (Sigma Aldrich, D5025), Hyaluronidase (Sigma Aldrich, H6254)

8. RBC red blood cell lysis buffer (Biolegend, 420302)

9. Cell staining buffer (Biolegend, 420201)

10. Human TruStain FcX™ (Fc Receptor Blocking Solution) (Biolegend, 422302)

11. Antibodies:

Anti-human CD45 antibody (Beckman, IM0782U), anti-human CD8 antibody (Beckman, IM0452U and Biolegend, 300911), anti-human CD4 antibody (Beckman, B16491), anti-human CD56 antibody (Beckman, IM2474U), anti-human CD16 antibody (Beckman, IM1238U), anti-human CD25 antibody (Beckman, IM0479U).

### Mouse

1. Species: Mus musculus
Strain: Advanced immunodeficiency mouse.
(NOD.Cg-Prkdc^{scid} Il2rg
Source: Trineo Biotechnology Co., LTD.
Sex: Female
Age at initiation of study: 6-8 weeks
Body weight range at start of study: 17-28 g

2. Numbering and identification: Each mouse was numbered by ear tag. The housing cage was identified by cage card with the information including study number, cage number, animal number, sex, dose level, etc.

Animal grouping: The mice were divided into six groups: G1 (Vehicle), G2 (OBI-3424), G3 (anti-hPD-1), G4 (anti-hPD-L1), G5 (OBI-3424+anti-hPD-1), G6 (OBI-3424+anti-hPD-L1). Each group contains five mice. The human hepatocellular carcinoma cell line HepG2 were subcutaneously inoculated to the advanced immune deficiency mice. Total of 30 mice were included in this study.

3. Reason for animal selection: According to the guidelines for nonclinical studies of anticancer pharmaceuticals in non-clinical safety studies of drug suggestion published by TFDA, animal xenograft tumor models can be used to evaluate the efficacy of new drug or new anticancer drugs. The commonly used mouse strains including BALB/c, C57BL/6, whereas BALB/c Nude, Nu/Nu and NOD/SCID mice are usually selected for evaluating the anti-tumor effect of desired drugs. The strains are managed on a global basis with well-known genetic and breed background, which can provide a valuable insight into functional significance of a proper reaction in human body.

4. Period of acclimatization: The mice were acclimated for at least 3 days before randomization. Clinical observations and body weight measurements was performed during acclimation period. The animals did not show any signs of illness or altered behavior during this period.

5. Animal housing condition: The mice were housed in individually ventilated cages (IVC) with the sterile bedding (10054, Andersons, USA) in a controlled environment with temperature 22±3° C., relative humidity 50±20% and 12/12 hr light/dark cycle. The food (LabDiet 5010, PMI, USA) and water (sterile RO water) were provided ad libitum throughout the whole study period.

6. Randomization: All animals were weighed, and the healthy conditions were observed prior to study. Animals without abnormal clinical signs were selected in the experiment. The healthy animals were randomized into different groups without significant difference in the body weight between groups. The weight variation of the animals should not exceed ±20% of the mean body weight. The procedure followed the standards of laboratory animal practices.

7. IACUC approval number: IACUC-2020-SH-016

### Equipment

Cell culture incubator (Shel Lab/3552)
Biosafety cabinet (BAKER/SG604)
Electronic balance (PRECISA/XS 225A-SCS)
Pipettes (Thermo/Finnpipette F1)
Isolated positive/negative pressure validated cage housing system (Allentown/NEXGEN)
Analytical balance (PRECISA/XS3250C-SCS)
Animal euthanasia equipment (Forward Biotech Supply)
Flow Cytometer (Beckman Coulter/Navios EX)
Vernier (Mitutoyo/CD-6″ ASX)

### Method

### Experimental Design

1. Sampling: The experimental design, experimental groups, doses and volume of injection, route of administration and animal numbers are listed in Table 1.

TABLE 1
Dosing regimen and sampling

| | Route and | | | |
|---|---|---|---|---|
| | adminis- | Dose of | Volume of | |
| | tration of | injection | injection | Animal |
| Groups | test item | (mg/kg) | (mg/kg) | No. |
| G1: Vehicle | IV and IP | N/A | 5 + 10 | 5 |
| G2: OBI-3424 | IV | 1 | 5 | 5 |
| G3: anti-hPD-1 | IP | 10 (20 from D 15*) | 5 | 5 |
| G4: anti-hPD-L1 | IP | 10 (20 from D 15*) | 10 | 5 |
| G5: OBI-3424 + | IV and IP | 10 (20 from D 15*) | 5 + 10 | 5 |
| anti-hPD-1 | | | | |
| G6: OBI-3424 + | IV and IP | 1 + 10 (20 from | 5 + 10 | 5 |
| anti-hPD-L1 | | D 15*) | | |

Intravenous injection (IV): Volume of injection is 5 mL/kg
Intraperitoneal injection (IP): Volume of injection is 10 mL/kg
*Dosage of intraperitoneal injection was changed to 20 mg/kg from Day 15.

2. Establishment of xenograft mouse model

2.1. Animal hair removal: Prior to the injection of human hepatocellular carcinoma cell line HepG2, the hair on right flank only was removed by clipping.

2.2. Subcutaneous inoculation of tumor cells: 1×10⁷ HepG2 cells were pre-mixed with 0.25×10⁷ hPBMC (cell number ratio 4:1) prior to the mixture of Matrigel (volume ratio 1:1) (Corning, Cat. No.: 354248, Lot No.: 8228001). The subcutaneous injection volume was 200 L/mouse.

3. Route and administration of test article:

3.1. The test item anti-hPD-1 antibody or reference items were intraperitoneally injected to the mice on Day 8 after the inoculation of tumor cells. The injection was performed using insulin syringe with the dosage of 10 mg/kg (Dosage of injection was changed to 20 mg/kg from Day 15) and injection volume of 10 mL/kg. The test item anti-hPD-1 antibody was continuously administered on Day 8, 11, 15, 18, 22, 25, 29 and 32 for G3 and G5. The reference item was administered for G1. The procedure followed the standards of sample administration.

3.2. The test item anti-hPD-L1 antibody or reference items were intraperitoneally injected to the mice on Day 8 after the inoculation of tumor cells. The injection was performed using insulin syringe with the dosage of 10 mg/kg (Dosage of injection was changed to 20 mg/kg from Day 15) and injection volume of 10 mL/kg. The test item anti-hPD-L1 was continuously administered on Day 8, 11, 15, 18, 22, 25, 29 and 32 for G4 and G6. The reference item was administered for G1. The procedure followed the standards of sample administration.

3.3. The test item OBI-3424, reference items were intravenously injected to the mice on Day 14 after the inoculation of tumor cells. The injection was performed using insulin syringe with the dosage of 1 mg/kg and injection volume of 5 mL/kg. The test item OBI-3424 was continuously administered on Day 14, 21, 28 and 35 for G2, G5 and G6. The reference item was administered for G1. The procedure followed the standards of sample administration.

3.4. Test item or reference item preparation:

Before administration, the test items were diluted by reference item. The solution concentration of test item OBI-3424 was 0.2 mg/mL, test item anti-hPD-1 and test item anti-hPD-L1 were 1 mg/mL (2 mg/mL from Day 15).

4. Body weight measurement:

The measurement was performed from the next day of the inoculation. The animal body weights were measured and recorded twice per week.

5. Tumor diameter measurement:

The measurement was performed from the next day of the inoculation. The tumor volumes were measured and recorded twice a week (Mon., Thur.). Tumor volume was calculated by ellipsoid equation according to the records ((major axis×minor axis×minor axis)×(π/6)).

6. Tumor growth inhibition ratio calculation:

Tumor volumes were used to calculate tumor growth inhibition (TGI) rates according to the following formula: TGI (%)=[1−(Ti−T0)/(Ci−C0)]×100, where Ti and Ci indicate the mean tumor volume in the treatment groups and vehicle group at the end of the experiment (Day 35). Whereas, T0 and C0 indicate the mean tumor volumes in the treatment group and vehicle group at the beginning of the experiment (Day 1).

7. Blood sampling:

Submandibular blood sample was collected at end point. At the sacrifice, blood sample could be collected using cardiac puncture. The collected blood sample was centrifuged 15 minutes in 4±2° C. and 1500×g to separate serum and pellet. The upper serum was collected and stored at a temperature below −70° C. The procedure followed the standards of animal blood sampling.

8. Determination of the end point of study:

The study was ended at Day 36.

9. Tumor resection:

Mice were sacrificed by CO₂ euthanasia at the end of the study duration, and the connective tissue around tumor was resected. The tumor samples were then removed and weighed. The half tissue was fixed in 10% formaldehyde and then embedded in paraffin; the other half was prepared for the isolation of tumor-infiltrating lymphocytes (TILs).

10. Isolation of tumor-infiltrating lymphocytes (TILs):

The half of mouse tumors were dissected into smaller fragments using scalpels and then digested with a cocktail of collagenase, DNase I, and Hyaluronidase (Collagenase #C5138, DNase I #D5025, Hyaluronidase #H6254, SigmaAldrich) for at least 2 hours. Tumor digests were then passed through a 70 m mesh cell strainer (Falcon #352350) using an syringe plunger and washed with PBS. Cells were treated with RBC red blood cell lysis buffer (Biolegend #420302), and single-cell suspensions for flow cytometry were prepared.

11. Flow cytometry analysis of TIL populations:

Cells were washed with staining buffer (Biolegend #420201), resuspended in staining buffer containing Fc Receptor Blocking Solution (Biolegend #422302), and incubated for 15 minutes at 4° C. Cells were stained with the fluorescently conjugated surface antibodies and incubated for 30 minutes at 4° C., and then resuspended in staining buffer for flow cytometric analysis. Flow cytometry was done using a Navios EX Flow Cytometer (Beckman Coulter). Data were analyzed using the Kaluza Analysis Software (Beckman Coulter).

12. Statistical analysis:

Results were presented as Mean and standard error of the mean (Mean±SEM). Comparisons of all data collected for each treatment group with concurrent negative control data was calculated using Student's t-test (Microsoft Excel, 2007). P≤0.05 is considered as significance.

### Result

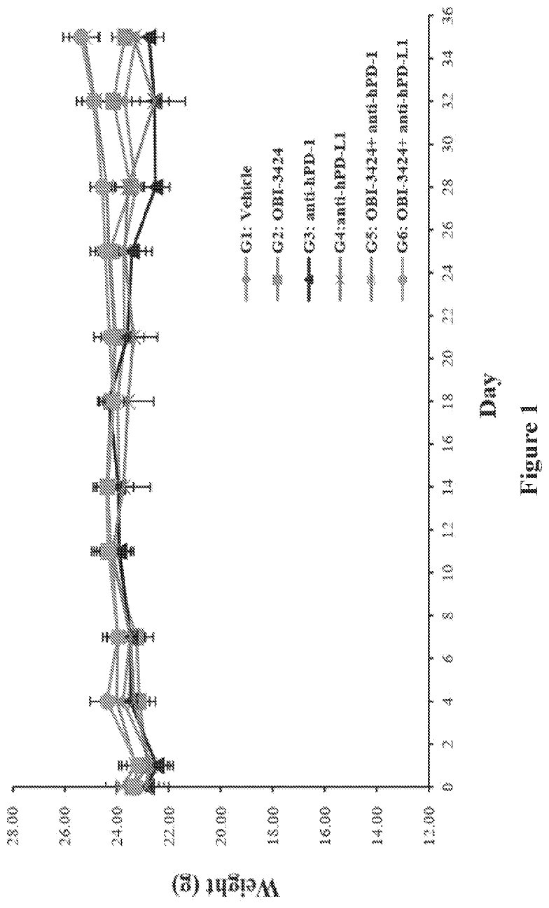

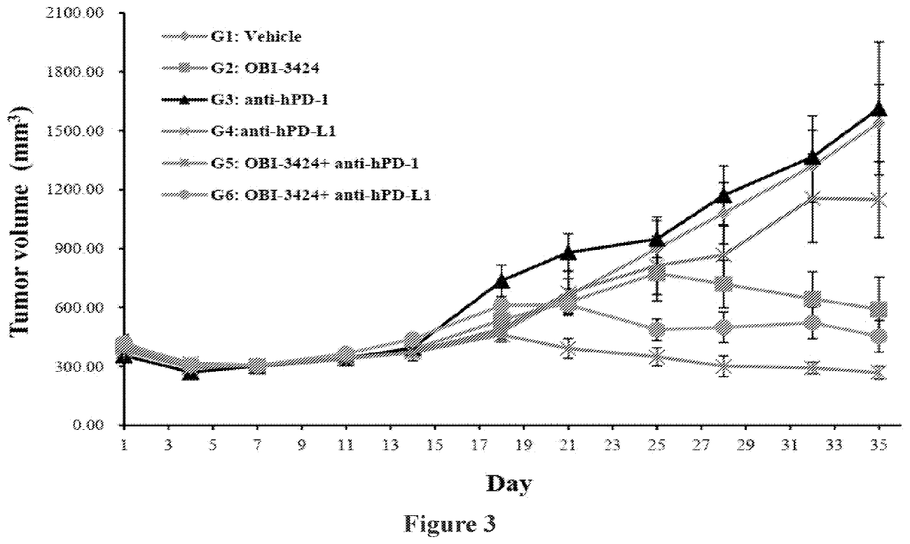

A summary of group body weights was presented in Table 2. There was no statistically significant difference was observed in the mean body weight among groups at the beginning of study. At the end point of study, the body weight of G5 (OBI-3424+anti-hPD-1) and G6 (OBI-3424+anti-hPD-L1) had slightly increased compared with other groups (FIG. 1 and Table 2). The tumor response was examined from different test items, the mean tumor responses were recorded at day 1, 4, 7, 11, 14, 18, 21, 25, 28, 32, and 35 (FIG. 3 and Table 3).

TABLE 2
Summary of body weights

| | Animal | Body Weight (g) | | | | | | | | | | | |
|---|---|---|---|---|---|---|---|---|---|---|---|---|---|
| Groups | No. | Day 0 | Day 1 | Day 4 | Day 7 | Day 11 | Day 14 | Day 18 | Day 21 | Day 25 | Day 28 | Day 32 | Day 35 |
| G1: | MI039 | 24.99 | 24.93 | 25.83 | 25.32 | 25.81 | 24.73 | 24.63 | 24.55 | 24.60 | 23.64 | 24.15 | 23.62 |
| Vehicle | MI047 | 20.78 | 20.80 | 21.81 | 22.05 | 22.97 | 23.69 | 23.28 | 23.07 | 22.91 | 22.23 | 23.04 | 22.58 |
| | MI049 | 23.24 | 22.25 | 23.62 | 22.65 | 24.47 | 24.98 | 24.70 | 23.99 | 23.91 | 23.77 | 24.10 | 23.87 |
| | MI057 | 22.07 | 22.32 | 23.10 | 23.14 | 22.35 | 21.86 | 23.11 | 23.13 | 23.22 | 23.31 | 23.02 | 22.01 |
| | MI059 | 23.33 | 22.89 | 24.40 | 24.24 | 24.12 | 24.30 | 24.12 | 23.80 | 24.02 | 23.35 | 24.16 | 24.20 |
| | Mean | 22.88 | 22.64 | 23.75 | 23.48 | 23.94 | 23.91 | 23.97 | 23.71 | 23.73 | 23.26 | 23.69 | 23.26 |
| | SEM | 0.70 | 0.67 | 0.67 | 0.58 | 0.60 | 0.56 | 0.33 | 0.28 | 0.30 | 0.27 | 0.27 | 0.41 |
| G2: OBI- | MI035 | 24.96 | 22.46 | 22.37 | 22.47 | 23.94 | 24.07 | 24.35 | 24.16 | 24.25 | 23.76 | 24.05 | 24.05 |
| 3424 | MI036 | 24.90 | 25.09 | 24.64 | 24.34 | 25.49 | 25.93 | 25.50 | 25.46 | 26.12 | 24.92 | 24.28 | 23.62 |
| | MI040 | 22.27 | 22.27 | 23.03 | 23.05 | 23.21 | 23.33 | 22.61 | 22.60 | 22.64 | 20.15 | 22.28 | 21.87 |
| | MI051 | 22.28 | 22.08 | 22.64 | 23.08 | 25.15 | 25.28 | 24.90 | 24.19 | 24.16 | 24.19 | 25.24 | 24.53 |
| | MI053 | 22.68 | 22.05 | 23.02 | 23.25 | 23.92 | 23.25 | 23.80 | 23.77 | 24.18 | 24.28 | 24.80 | 24.42 |
| | Mean | 23.42 | 22.79 | 23.14 | 23.24 | 24.34 | 24.37 | 24.23 | 24.04 | 24.27 | 23.46 | 24.13 | 23.70 |
| | SEM | 0.62 | 0.58 | 0.39 | 0.31 | 0.42 | 0.53 | 0.49 | 0.46 | 0.55 | 0.85 | 0.51 | 0.48 |
| G3: anti-hPD-1 | MI031 | 24.48 | 23.77 | 24.37 | 24.33 | 25.23 | 25.73 | 25.52 | 25.31 | 25.35 | 24.05 | 23.93 | 24.51 |
| | MI041 | 22.83 | 22.64 | 23.22 | 23.26 | 23.13 | 23.05 | 24.00 | 23.64 | 23.30 | 21.35 | 21.55 | 21.67 |
| | MI055 | 22.77 | 22.39 | 23.69 | 23.03 | 22.86 | 22.58 | 23.68 | 23.07 | 23.44 | 22.97 | 23.13 | 23.02 |
| | MI058 | 22.35 | 22.37 | 23.44 | 23.66 | 24.79 | 24.78 | 24.66 | 24.39 | 24.30 | 23.07 | 23.22 | 23.30 |
| | MI060 | 21.75 | 21.18 | 22.64 | 23.10 | 23.48 | 23.54 | 23.64 | 21.55 | 20.71 | 21.15 | 20.92 | 21.34 |
| | Mean | 22.84 | 22.47 | 23.47 | 23.48 | 23.90 | 23.94 | 24.30 | 23.59 | 23.42 | 22.52 | 22.55 | 22.77 |
| | SEM | 0.45 | 0.41 | 0.28 | 0.24 | 0.47 | 0.58 | 0.36 | 0.63 | 0.77 | 0.55 | 0.56 | 0.58 |
| G4: | MI037 | 23.68 | 23.15 | 23.83 | 23.75 | 24.89 | 25.06 | 24.70 | 24.14 | 24.47 | 23.71 | 23.83 | 23.41 |
| anti-hPD-L1 | MI046 | 25.61 | 25.88 | 26.01 | 26.73 | 26.45 | 26.71 | 26.09 | 26.11 | 25.74 | 25.42 | 25.58 | 24.98 |
| | MI048 | 23.03 | 22.46 | 23.49 | 23.02 | 23.97 | 23.84 | 24.20 | 24.05 | 24.60 | 22.64 | 22.43 | 22.69 |
| | MI052 | 21.27 | 21.36 | 21.62 | 21.92 | 22.76 | 22.29 | 22.27 | 21.02 | 22.60 | 21.81 | 18.37 | * |
| | MI062 | 20.75 | 20.78 | 21.66 | 22.00 | 22.78 | 20.81 | 20.56 | 21.51 | 21.07 | 24.27 | 22.52 | 22.32 |
| | Mean | 22.87 | 22.73 | 23.32 | 23.48 | 24.17 | 23.74 | 23.56 | 23.37 | 23.70 | 23.57 | 22.55 | 23.35 |
| | SEM | 0.87 | 8.89 | 0.81 | 8.88 | 0.70 | 1.03 | 0.97 | 0.94 | 0.83 | 0.63 | 1.19 | 0.59 |
| G5: | MI034 | 24.95 | 24.33 | 25.05 | 24.40 | 25.22 | 25.31 | 24.96 | 24.83 | 24.70 | 24.33 | 25.61 | 25.91 |
| OBI-3424 + | MI042 | 25.59 | 25.08 | 25.58 | 25.24 | 24.20 | 24.12 | 24.42 | 24.53 | 25.02 | 25.15 | 25.57 | 26.42 |
| anti-hPD-1 | MI050 | 22.28 | 22.34 | 23.28 | 23.74 | 25.05 | 25.31 | 24.89 | 25.38 | 25.09 | 25.13 | 25.75 | 26.23 |
| | MI054 | 23.20 | 23.02 | 23.81 | 23.75 | 22.96 | 23.02 | 22.93 | 22.61 | 23.68 | 23.46 | 23.66 | 23.62 |
| | MI056 | 22.96 | 21.62 | 22.27 | 22.67 | 23.69 | 24.00 | 23.47 | 23.02 | 23.08 | 23.81 | 23.66 | 24.08 |
| | Mean | 23.80 | 23.28 | 24.00 | 23.96 | 24.22 | 24.35 | 24.13 | 24.07 | 24.31 | 24.38 | 24.85 | 25.25 |
| | SEM | 0.63 | 0.63 | 0.60 | 0.42 | 0.42 | 0.44 | 0.40 | 0.54 | 0.40 | 0.34 | 0.49 | 0.58 |
| G6: | MI033 | 24.49 | 24.60 | 25.89 | 25.25 | 25.37 | 24.64 | 24.78 | 24.95 | 25.59 | 25.71 | 25.99 | 26.47 |
| OBI-3424 + | MI038 | 22.81 | 22.96 | 24.01 | 24.22 | 24.80 | 24.30 | 24.40 | 24.38 | 24.70 | 24.18 | 24.12 | 24.45 |
| anti-hPD-L1 | MI043 | 21.17 | 21.04 | 22.06 | 21.83 | 21.70 | 22.36 | 22.13 | 22.13 | 22.31 | 23.00 | 22.71 | 23.24 |
| | MI044 | 24.26 | 23.76 | 25.16 | 24.35 | 25.05 | 25.46 | 24.03 | 24.15 | 24.37 | 24.80 | 25.51 | 25.78 |
| | MI045 | 23.88 | 23.67 | 24.74 | 24.22 | 24.62 | 25.02 | 25.30 | 25.75 | 25.26 | 25.12 | 26.14 | 26.98 |
| | Mean | 23.32 | 23.21 | 24.37 | 23.97 | 24.31 | 24.36 | 24.13 | 24.27 | 24.45 | 24.56 | 24.89 | 25.38 |
| | SEM | 0.61 | 0.60 | 0.65 | 0.57 | 0.66 | 0.54 | 0.54 | 0.60 | 0.57 | 0.46 | 0.65 | 0.68 |

* MI052 was found dead on Day 34.

TABLE 3
Summary of tumor volumes and weights

| | | | | | | | | | | | | | Tumor | |
|---|---|---|---|---|---|---|---|---|---|---|---|---|---|---|
| | | | | | | | | | | | | | weight | |
| | Animal | Tumor volume (mm³) | | | | | | | | | | | (mg) | TGI |
| Groups | No. | D 1 | D 4 | D 7 | D 11 | D 14 | D 18 | D 21 | D 25 | D 28 | D 32 | D 35 | D 35 | (%) |
| G1: | MI039 | 273.66 | 265.50 | 334.42 | 388.44 | 462.39 | 600.99 | 954.55 | 1033.73 | 1246.21 | 1698.35 | 1833.05 | 1064.10 | N/A |
| Vehicle | MI047 | 300.06 | 222.20 | 246.06 | 268.86 | 333.72 | 538.18 | 716.42 | 1405.89 | 1582.38 | 1771.38 | 2073.85 | 1704.60 | |
| | MI049 | 322.78 | 280.12 | 289.67 | 292.95 | 321.52 | 372.96 | 523.55 | 557.61 | 670.58 | 796.22 | 940.35 | 638.90 | |
| | MI057 | 482.26 | 362.35 | 332.03 | 382.50 | 404.25 | 465.56 | 639.62 | 955.72 | 991.31 | 1159.28 | 1357.04 | 995.80 | |
| | MI059 | 576.81 | 357.00 | 292.38 | 349.42 | 380.15 | 405.82 | 464.71 | 544.77 | 906.23 | 1171.66 | 1488.24 | 933.90 | |
| | Mean | 391.11 | 297.43 | 298.91 | 336.43 | 380.41 | 476.70 | 659.77 | 899.54 | 1079.34 | 1319.38 | 1538.51 | 1067.46 | |
| | SEM | 58.97 | 27.15 | 16.25 | 23.93 | 25.43 | 41.91 | 85.77 | 161.30 | 155.84 | 182.90 | 195.78 | 175.01 | |
| G2: OBI- | MI035 | 336.94 | 248.60 | 247.70 | 272.45 | 316.87 | 443.46 | 495.51 | 530.21 | 534.09 | 458.78 | 326.08 | 187.80 | 83.13 |
| 3424 | MI036 | 416.45 | 319.17 | 329.31 | 403.25 | 450.69 | 595.27 | 703.20 | 1087.59 | 929.53 | 802.95 | 875.83 | 442.00 | |
| | MI040 | 374.19 | 317.11 | 337.59 | 346.18 | 410.20 | 558.01 | 847.55 | 1133.30 | 1088.12 | 1097.24 | 1092.67 | 575.30 | |
| | MI051 | 431.65 | 274.75 | 282.91 | 337.46 | 361.93 | 491.94 | 487.42 | 479.83 | 505.34 | 313.47 | 325.92 | 74.00 | |
| | MI053 | 426.18 | 316.40 | 293.88 | 361.50 | 385.75 | 588.12 | 603.56 | 635.01 | 537.45 | 543.00 | 332.59 | 93.10 | |
| | Mean | 397.08 | 295.20 | 298.28 | 344.17 | 385.09 | 535.36 | 627.45 | 773.19 | 718.91 | 643.09 | 590.62 | 274.44 | |
| | SEM | 18.11 | 14.31 | 16.31 | 21.19 | 22.50 | 29.34 | 67.74 | 140.13 | 121.11 | 138.64 | 164.32 | 99.79 | |
| G3: anti- | MI031 | 317.46 | 236.70 | 257.47 | 308.80 | 397.01 | 693.99 | 881.79 | 1085.08 | 1583.17 | 1923.48 | 2446.76 | 2447.20 | −9.69 |
| hPD-1 | MI041 | 272.82 | 209.94 | 296.62 | 308.98 | 339.27 | 629.12 | 1133.34 | 1200.68 | 1453.15 | 1801.67 | 2416.49 | 2501.20 | |
| | MI055 | 354.07 | 270.30 | 282.75 | 291.53 | 334.80 | 515.62 | 545.25 | 648.40 | 782.93 | 970.79 | 1023.97 | 447.30 | |
| | MI058 | 458.23 | 341.42 | 343.00 | 412.75 | 473.75 | 891.77 | 904.48 | 901.39 | 1044.52 | 1211.89 | 1248.89 | 655.20 | |
| | MI060 | 371.40 | 288.50 | 328.21 | 375.46 | 423.96 | 944.97 | 935.49 | 904.19 | 991.40 | 923.05 | 930.75 | 281.50 | |
| | Mean | 354.80 | 269.37 | 301.61 | 339.50 | 393.76 | 735.09 | 880.07 | 947.95 | 1171.03 | 1366.17 | 1613.37 | 1266.48 | |
| | SEM | 30.89 | 22.54 | 15.41 | 23.27 | 26.24 | 80.53 | 94.85 | 93.94 | 149.72 | 209.37 | 338.07 | 496.67 | |
| G4: | MI037 | 311.93 | 275.12 | 258.52 | 261.37 | 274.59 | 334.71 | 560.79 | 596.92 | 636.51 | 844.25 | 1122.20 | 960.10 | 35.60 |
| anti- | MI046 | 470.43 | 344.51 | 365.09 | 379.77 | 444.66 | 599.46 | 759.83 | 849.43 | 918.32 | 1330.79 | 1513.23 | 1437.50 | |
| hPD-L1 | MI048 | 376.06 | 275.94 | 275.91 | 328.38 | 353.38 | 481.58 | 589.05 | 897.19 | 949.94 | 1161.94 | 1337.47 | 1190.20 | |
| | MI052 | 413.00 | 384.63 | 326.38 | 445.55 | 490.86 | 624.77 | 1049.86 | 1294.64 | 1343.20 | 1884.53 | * | *963.00 | |
| | MI062 | 477.14 | 300.15 | 296.62 | 299.06 | 293.86 | 415.39 | 417.12 | 426.57 | 486.86 | 557.69 | 621.65 | 455.10 | |
| | Mean | 409.71 | 316.07 | 304.50 | 342.83 | 371.47 | 491.18 | 675.33 | 812.95 | 866.97 | 1155.84 | 1148.64 | *1010.73 | |
| | SEM | 30.77 | 21.27 | 18.90 | 32.15 | 42.04 | $4.72 | 108.32 | 147.72 | 147.29 | 225.44 | 193.00 | 209.29 | |
| G5: OBI- | MI034 | 255.56 | 235.32 | 259.12 | 316.52 | 319.27 | 406.87 | 333.76 | 271.54 | 259.90 | 317.15 | 388.43 | 124.30 | 109.71 |
| 3424 + | MI042 | 365.53 | 303.74 | 314.43 | 364.96 | 412.55 | 508.29 | 322.13 | 314.43 | 298.78 | 320.28 | 245.14 | 53.70 | |
| anti- | MI050 | 450.24 | 376.72 | 371.44 | 372.22 | 438.22 | 579.37 | 570.71 | 493.70 | 503.36 | 367.28 | 242.18 | 102.40 | |
| hPD-1 | MI054 | 445.96 | 267.88 | 273.95 | 314.86 | 334.05 | 384.14 | 295.90 | 243.93 | 211.39 | 196.54 | 198.00 | 53.10 | |
| | MI056 | 376.86 | 279.26 | 300.66 | 327.56 | 347.38 | 423.68 | 432.55 | 417.39 | 229.54 | 257.99 | 263.40 | 65.70 | |
| | Mean | 378.83 | 292.58 | 303.92 | 339.23 | 370.30 | 460.47 | 391.01 | 348.20 | 300.59 | 291.85 | 267.43 | 79.84 | |
| | SEM | 35.34 | 23.74 | 19.47 | 12.24 | 23.28 | 36.38 | 50.54 | 46.82 | 52.81 | 29.47 | 32.10 | 14.29 | |
| G6: OBI- | MI033 | 383.35 | 290.41 | 302.58 | 412.69 | 451.18 | 568.41 | 452.77 | 375.48 | 426.92 | 519.39 | 396.47 | 147.70 | 97.11 |
| 3424 + | MI038 | 286.46 | 235.58 | 260.09 | 326.89 | 435.04 | 570.91 | 615.63 | 595.06 | 735.67 | 793.12 | 773.46 | 429.20 | |
| antihPD-L1 | MI043 | 516.08 | 320.84 | 267.89 | 306.21 | 340.88 | 695.17 | 687.48 | 641.22 | 565.46 | 512.11 | 366.43 | 132.80 | |
| | MI044 | 418.50 | 332.14 | 388.79 | 389.37 | 484.13 | 672.17 | 688.96 | 406.06 | 259.81 | 281.58 | 341.67 | 86.90 | |
| | MI045 | 493.81 | 349.65 | 306.13 | 389.67 | 478.67 | 548.76 | 623.49 | 413.49 | 504.75 | 501.25 | 385.72 | 145.60 | |
| | Mean | 419.64 | 305.72 | 308.09 | 364.96 | 437.98 | 611.08 | 613.66 | 486.26 | 498.52 | 521.49 | 452.75 | 188.44 | |
| | SEM | 41.15 | 20.01 | 22.83 | 20.48 | 25.88 | 30.10 | 43.07 | 54.70 | 78.38 | 81.19 | 80.72 | 61.18 | |

*MI052 was found dead and tumor wight was recorded on Day 34.
MI052 was not included in the mean tumor weight statistical calculation.

Firstly, we examined the effects of all test items from G1 (Vehicle), G2 (OBI-3424), G3 (anti-hPD-1) and G4 (anti-hPD-L1) on tumor response. Significantly reduced in mean tumor volume was observed in the presence of OBI-3424 at Day 35 (G1 Vehicle: 1538.51±195.78 mm³, G2 OBI-3424: 590.62±164.32 mm³, p=0.003<0.05). No statistically significant difference was observed in the treatment of anti-hPD-1 and antihPD-L1 at day 35 (G1 Vehicle: 1538.51±195.78 mm³, G3 anti-hPD-1: 1613.37±338.07 mm³, G4 anti-hPD-L1: 1148.64±193.00 mm³).

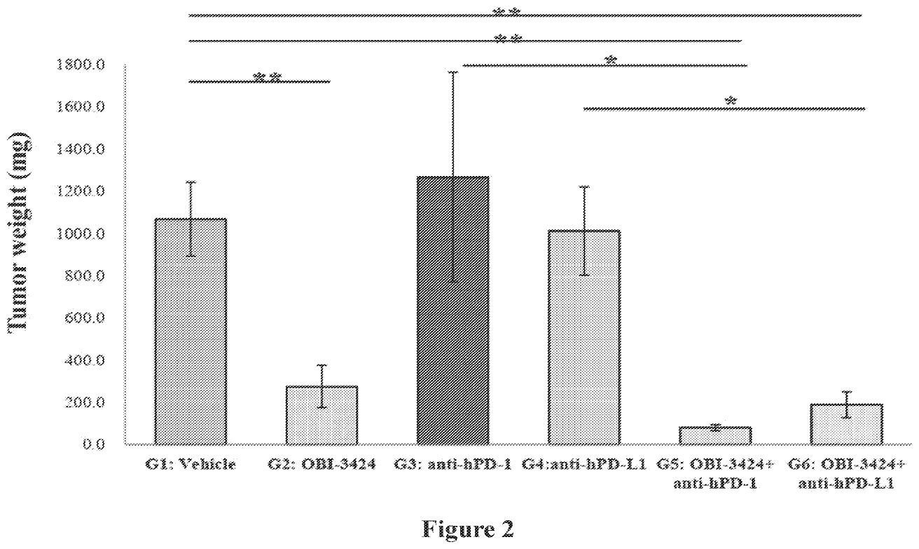

In addition, with combined treatment of G5 (anti-hPD-1+OBI-3424) and G6 (antihPD-L1+OBI-3424), the results showed significant decreased in mean tumor volume at Day 35 (G1 Vehicle: 1538.51±195.78 mm³, G5 anti-hPD-1+OBI-3424: 267.43±32.10 mm³, p=0.0001<0.001, G6 anti-hPD-L1+OBI-3424: 452.75±80.72 mm³, p=0.0005<0.001), meanwhile there was significant decreased in mean tumor volume at Day 35 between G3 and G5 (G3 anti-hPD-1: 1613.37±338.07 mm³, G5 anti-hPD-1+OBI-3424: 267.43±32.10 mm³, p=0.002<0.05). Furthermore, tumor volume at Day 35 between G4 and G6 also showed significant decreased in mean tumor volume at Day 35 (G4 anti-hPD-L1: 1148.64±193.00 mm³, G6 anti-hPD-L1+OBI-3424: 452.75±80.72 mm³, p=0.004<0.05) (FIG. 3 and Table 3). A similar trend was observed on the tumor weights (FIG. 2 and Table 3). Besides, one mouse of G4 (anti-hPD-L1) was found dead on Day 34, the tumor weight was recorded at the same day.

The tumor-infiltrating lymphocytes (TILs) were isolated from fresh tumor tissue, and measured the expression levels of surface markers CD45, CD4, CD8, CD56, CD16, CD25, PD-1 and PD-L1 among groups by flow cytometer. The numerical data for individual mice were presented in Tables 4-7. It indicated that CD8⁺ cytotoxic T cells (CTL) cells population were significantly elevated in tumors after OBI-3424 treatment (G2) when compared with vehicle, but this increasement was not observed in the tumors after anti-PD-1 (G3) or anti-PD-L1 (G4) treatment. However, in the tumors after OBI-3424+anti-PD-1 (G5) and OBI-3424+anti-PD-L1 (G6) treatments, the CTL population were found to be elevated significantly in both groups. This suggested that the increased CTL population were caused by OBI-3424 not anti-PD-1 or anti-PD-L1 treatment. Moreover, CD4⁺ T helper cells population were significantly increased in the tumors of G5 and G6. NIK cell population were not significant differences between vehicle and each treatment group.

TABLE 4
Summary for percentage of CTL cells in TILs

| | | | | | CTL cells | | | CD25⁺ CTL cells | | | PD-1⁺ CTL cells | | |
|---|---|---|---|---|---|---|---|---|---|---|---|---|---|
| | | Cell | | | (CD45⁺CD8⁺) | | | (CD45⁺CD8⁺ CD25⁺) | | | (CD45⁺CD8⁺ PD-1⁺) | | |
| | | Conc. | Total | | | CD45⁺ | Cell | | CTL | Cell | | CTL | Cell |
| | Animal | ×(10⁵/ | cell | Viability | Total | Gated | count | Total | Gated | count | Total | Gated | count |
| Groups | No. | mL) | ×10⁶ | % | % | % | (×10⁵) | % | % | (×10⁵) | % | % | (×10⁵) |
| G1: | MI039 | 5.87 | 10.50 | 55.90 | 9.21 | 53.75 | 9.67 | 0.40 | 4.35 | 0.42 | 13.93 | 95.18 | 14.63 |
| Vehicle | MI047 | 8.63 | 13.10 | 65.88 | 10.71 | 41.07 | 14.03 | 0.35 | 3.27 | 0.46 | 10.06 | 90.88 | 13.18 |
| | MI049 | 5.16 | 12.30 | 41.95 | 6.62 | 51.92 | 8.14 | 0.85 | 12.84 | 1.05 | 5.85 | 80.80 | 7.20 |
| | MI057 | 5.53 | 9.62 | 57.48 | 7.90 | 37.24 | 7.60 | 0.07 | 0.89 | 0.07 | 20.46 | 95.45 | 19.68 |
| | MI059 | 12.30 | 19.00 | 64.74 | 14.06 | 37.94 | 26.71 | 0.19 | 1.35 | 0.36 | 13.78 | 90.03 | 26.18 |
| G2: OBI- | MI035 | 2.85 | 4.36 | 65.37 | 20.93 | 49.08 | 9.13 | 0.27 | 1.27 | 0.12 | 21.41 | 92.13 | 9.33 |
| 3424 | MI036 | 2.81 | 7.37 | 38.13 | 12.12 | 66.81 | 8.93 | 0.30 | 2.48 | 0.22 | 12.30 | 91.25 | 9.07 |
| | MI040 | 5.72 | 8.18 | 69.93 | 21.14 | 72.14 | 17.29 | 0.77 | 3.64 | 0.63 | 20.63 | 99.25 | 16.88 |
| | MI051 | 1.91 | 2.44 | 78.28 | 45.53 | 74.09 | 11.11 | 0.52 | 1.13 | 0.13 | 41.26 | 91.79 | 10.07 |
| | MI053 | 3.22 | 4.06 | 79.31 | 36.08 | 57.70 | 14.65 | 0.28 | 0.78 | 0.11 | 28.90 | 85.06 | 11.73 |
| G3: | MI031 | 20.80 | 30.40 | 68.42 | 9.30 | 36.10 | 28.27 | 0.26 | 2.80 | 0.79 | 10.83 | 89.36 | 32.92 |
| anti-hPD-1 | MI041 | 23.00 | 35.10 | 65.53 | 6.22 | 34.75 | 21.83 | 0.10 | 1.53 | 0.35 | 9.53 | 84.37 | 33.45 |
| | MI055 | 6.17 | 9.53 | 64.74 | 21.30 | 75.47 | 20.30 | 0.65 | 3.05 | 0.62 | 15.71 | 66.70 | 14.97 |
| | MI058 | 7.72 | 12.90 | 59.84 | 11.17 | 53.85 | 14.41 | 0.84 | 7.48 | 1.08 | 10.88 | 66.59 | 14.04 |
| | MI060 | 4.45 | 6.79 | 65.54 | 24.91 | 55.75 | 16.91 | 0.41 | 1.65 | 0.28 | 14.41 | 57.38 | 9.78 |
| G4: | MI037 | 8.66 | 12.20 | 70.98 | 14.00 | 57.51 | 17.08 | 0.27 | 1.93 | 0.33 | 17.16 | 97.44 | 20.94 |
| anti-hPD-L1 | MI046 | 15.00 | 22.00 | 68.18 | 18.51 | 57.17 | 40.72 | 0.30 | 1.59 | 0.66 | 16.84 | 88.98 | 37.05 |
| | MI048 | 11.60 | 17.70 | 65.54 | 11.85 | 56.56 | 20.97 | 0.21 | 1.77 | 0.37 | 12.16 | 94.63 | 21.52 |
| | MI062 | 3.28 | 7.22 | 45.43 | 16.01 | 70.33 | 11.56 | 0.63 | 3.90 | 0.45 | 16.68 | 98.61 | 12.04 |
| G5: | MI034 | 2.00 | 2.90 | 68.97 | 30.12 | 56.19 | 8.73 | 0.64 | 2.13 | 0.19 | 15.92 | 52.86 | 4.62 |
| OBI-3424 + | MI042 | 0.24 | 0.49 | 48.28 | 25.43 | 46.84 | 1.25 | 0.20 | 0.77 | 0.01 | 11.13 | 35.95 | 0.55 |
| anti-hPD-1 | MI050 | 1.71 | 2.91 | 58.76 | 20.78 | 59.78 | 6.05 | 0.44 | 2.09 | 0.13 | 11.90 | 52.33 | 3.46 |
| | MI054 | 0.89 | 1.21 | 73.88 | 31.55 | 63.27 | 3.82 | 0.58 | 1.84 | 0.07 | 16.88 | 48.79 | 2.04 |
| | MI056 | 2.06 | 3.13 | 65.81 | 30.09 | 55.23 | 9.42 | 0.37 | 1.23 | 0.12 | 12.41 | 39.23 | 3.88 |
| G6: | MI033 | 2.16 | 3.20 | 67.50 | 27.93 | 65.90 | 8.94 | 0.30 | 1.07 | 0.10 | 25.02 | 87.30 | 8.01 |
| OBI-3424 + | MI038 | 4.46 | 5.69 | 78.38 | 31.74 | 49.21 | 18.06 | 0.39 | 1.23 | 0.22 | 30.71 | 97.65 | 17.47 |
| anti-hPD-L1 | MI043 | 3.20 | 4.50 | 71.11 | 40.03 | 69.67 | 18.01 | 0.28 | 0.69 | 0.13 | 25.12 | 66.20 | 11.30 |
| | MI044 | 1.06 | 1.81 | 58.56 | 22.68 | 53.32 | 4.11 | 0.33 | 1.43 | 0.06 | 20.46 | 89.05 | 3.70 |
| | MI045 | 3.28 | 4.20 | 78.10 | 32.10 | 61.60 | 13.48 | 0.27 | 0.83 | 0.11 | 29.07 | 93.11 | 12.21 |

CD45⁺ Gated %= Gate CD45⁺ cells
Cell count (×10⁴)=Total cell × Total % × 10
CTL Gated %= Gate CD45⁺CD8⁺ cells

TABLE 5
Summary for percentage of TH cells in TILs

| | | | | | TH cells | | | CD25⁺ TH cells | | | PD-1⁺ TH cells | | |
|---|---|---|---|---|---|---|---|---|---|---|---|---|---|
| | | Cell | | | (CD45⁺CD4⁺) | | | (CD45⁺CD4⁺ CD25⁺) | | | (CD45⁺CD4⁺ PD-1⁺) | | |
| | | Conc. | Total | | | CD45⁺ | Cell | | TH | Cell | | TH | Cell |
| | Animal | ×(10⁵/ | cell | Viability | Total | Gated | count | Total | Gated | count | Total | Gated | count |
| Groups | No. | mL) | ×10⁶ | % | % | % | (×10⁵) | % | % | (×10⁵) | % | % | (×10⁵) |
| G1: | MI039 | 5.87 | 10.50 | 55.90 | 6.86 | 40.03 | 7.20 | 0.59 | 8.61 | 0.62 | 7.13 | 99.30 | 7.49 |
| Vehicle | MI047 | 8.63 | 13.10 | 65.88 | 15.84 | 60.74 | 20.75 | 0.77 | 4.86 | 1.01 | 14.43 | 98.23 | 18.90 |
| | MI049 | 5.16 | 12.30 | 41.95 | 5.60 | 43.88 | 6.89 | 0.58 | 10.28 | 0.71 | 5.47 | 98.20 | 6.73 |
| | MI057 | 5.53 | 9.62 | 57.48 | 11.34 | 53.43 | 10.91 | 0.39 | 3.40 | 0.38 | 15.83 | 99.94 | 15.23 |
| | MI059 | 12.30 | 19.00 | 64.74 | 17.72 | 47.82 | 33.67 | 0.77 | 4.35 | 1.46 | 18.40 | 99.00 | 34.96 |
| G2: OBI- | MI035 | 2.85 | 4.36 | 65.37 | 18.53 | 43.45 | 8.08 | 1.06 | 5.70 | 0.46 | 19.45 | 99.46 | 8.48 |
| 3424 | MI036 | 2.81 | 7.37 | 38.13 | 9.14 | 50.36 | 6.74 | 0.68 | 7.39 | 0.50 | 9.29 | 98.15 | 6.85 |
| | MI040 | 5.72 | 8.18 | 69.93 | 14.30 | 48.78 | 11.70 | 0.82 | 5.74 | 0.67 | 13.77 | 99.75 | 11.26 |
| | MI051 | 1.91 | 2.44 | 78.28 | 14.34 | 23.33 | 3.50 | 0.52 | 3.63 | 0.13 | 15.00 | 97.06 | 3.66 |
| | MI053 | 3.22 | 4.06 | 79.31 | 16.81 | 26.88 | 6.82 | 0.53 | 3.15 | 0.22 | 16.75 | 97.78 | 6.80 |
| G3: | MI031 | 20.80 | 30.40 | 68.42 | 19.18 | 74.49 | 58.31 | 0.71 | 3.70 | 2.16 | 20.78 | 96.11 | 63.17 |
| anti-hPD-1 | MI041 | 23.00 | 35.10 | 65.53 | 11.07 | 61.90 | 38.86 | 0.67 | 6.05 | 2.35 | 11.99 | 90.08 | 42.08 |
| | MI055 | 6.17 | 9.53 | 64.74 | 9.76 | 34.59 | 9.30 | 0.82 | 8.35 | 0.78 | 9.21 | 83.23 | 8.78 |
| | MI058 | 7.72 | 12.90 | 59.84 | 11.17 | 53.85 | 14.41 | 1.28 | 11.42 | 1.65 | 11.61 | 82.55 | 14.98 |
| | MI060 | 4.45 | 6.79 | 65.54 | 18.23 | 40.81 | 12.38 | 0.45 | 2.44 | 0.31 | 15.49 | 85.27 | 10.52 |
| G4: | MI037 | 8.66 | 12.20 | 70.98 | 9.82 | 40.34 | 11.98 | 1.19 | 12.12 | 1.45 | 10.85 | 99.40 | 13.24 |
| anti-hPD-L1 | MI046 | 15.00 | 22.00 | 68.18 | 12.42 | 38.36 | 27.32 | 1.34 | 10.75 | 2.95 | 11.67 | 99.02 | 25.67 |
| | MI048 | 11.60 | 17.70 | 65.54 | 6.81 | 32.51 | 12.05 | 1.01 | 14.76 | 1.79 | 6.66 | 99.40 | 11.79 |
| | MI062 | 3.28 | 7.22 | 45.43 | 15.92 | 69.93 | 11.49 | 1.14 | 7.16 | 0.82 | 15.38 | 99.19 | 11.10 |
| G5: | MI034 | 2.00 | 2.90 | 68.97 | 31.40 | 58.59 | 9.11 | 1.49 | 4.73 | 0.43 | 29.96 | 89.25 | 8.69 |
| OBI-3424 + | MI042 | 0.24 | 0.49 | 48.28 | 29.46 | 54.25 | 1.45 | 0.60 | 2.02 | 0.03 | 28.73 | 86.08 | 1.42 |
| anti-hPD-1 | MI050 | 1.71 | 2.91 | 58.76 | 14.65 | 42.15 | 4.26 | 0.94 | 6.42 | 0.27 | 13.65 | 87.05 | 3.97 |
| | MI054 | 0.89 | 1.21 | 73.88 | 22.65 | 45.42 | 2.74 | 1.62 | 7.13 | 0.20 | 20.44 | 83.31 | 2.47 |
| | MI056 | 2.06 | 3.13 | 65.81 | 24.15 | 44.32 | 7.56 | 0.85 | 3.50 | 0.27 | 22.53 | 87.16 | 7.05 |
| G6: | MI033 | 2.16 | 3.20 | 67.50 | 14.36 | 33.88 | 4.60 | 0.56 | 3.90 | 0.18 | 15.43 | 98.75 | 4.94 |
| OBI-3424 + | MI038 | 4.46 | 5.69 | 78.38 | 43.37 | 67.24 | 24.68 | 3.95 | 9.11 | 2.25 | 41.44 | 99.72 | 23.58 |
| antihPD-L1 | MI043 | 3.20 | 4.50 | 71.11 | 19.38 | 33.73 | 8.72 | 0.53 | 2.71 | 0.24 | 20.71 | 99.00 | 9.32 |
| | MI044 | 1.06 | 1.81 | 58.56 | 19.02 | 44.72 | 3.44 | 0.94 | 4.94 | 0.17 | 21.16 | 99.18 | 3.83 |
| | MI045 | 3.28 | 4.20 | 78.10 | 20.28 | 38.91 | 8.52 | 0.59 | 2.91 | 0.25 | 21.10 | 99.67 | 8.86 |

CD45⁺ Gated % = Gate CD45⁺ cells
Cell count (×10⁴) = Total cell × Total % × 10
TH Gated % = Gate CD45⁺CD4⁺ cells

TABLE 6
Summary for percentage of NK cells in TILs

| | | | | | NK cells | | | CD16⁺ NK cells | | | PD-1⁺ NK cells | | |
|---|---|---|---|---|---|---|---|---|---|---|---|---|---|
| | | Cell | | | (CD45⁺CD56⁺) | | | (CD45⁺CD56⁺ CD16⁺) | | | (CD45⁺CD56⁺ PD-1⁺) | | |
| | | Conc. | Total | | | CD45⁺ | Cell | | NK | Cell | | NK | Cell |
| | Animal | ×(10⁵/ | cell | Viability | Total | Gated | count | Total | Gated | count | Total | Gated | count |
| Groups | No. | mL) | ×10⁶ | % | % | % | (×10⁵) | % | % | (×10⁵) | % | % | (×10⁵) |
| G1: | MI039 | 5.87 | 10.50 | 55.90 | 1.26 | 9.08 | 1.32 | 0.16 | 12.30 | 0.17 | 1.89 | 99.74 | 1.98 |
| Vehicle | MI047 | 8.63 | 13.10 | 65.88 | 2.62 | 9.86 | 3.43 | 1.01 | 38.43 | 1.32 | 1.68 | 94.10 | 2.20 |
| | MI049 | 5.16 | 12.30 | 41.95 | 2.78 | 23.40 | 3.42 | 0.69 | 24.82 | 0.85 | 1.04 | 66.77 | 1.28 |
| | MI057 | 5.53 | 9.62 | 57.48 | 0.74 | 3.67 | 0.71 | 0.26 | 34.46 | 0.25 | 1.91 | 100.00 | 1.84 |
| | MI059 | 12.30 | 19.00 | 64.74 | 0.72 | 1.96 | 1.37 | 0.18 | 24.48 | 0.34 | 3.36 | 94.12 | 6.38 |
| G2: OBI- | MI035 | 2.85 | 4.36 | 65.37 | 2.47 | 6.06 | 1.08 | 0.39 | 15.62 | 0.17 | 3.26 | 88.81 | 1.42 |
| 3424 | MI036 | 2.81 | 7.37 | 38.13 | 1.62 | 9.96 | 1.19 | 0.21 | 12.65 | 0.15 | 1.51 | 88.82 | 1.11 |
| | MI040 | 5.72 | 8.18 | 69.93 | 1.15 | 4.16 | 0.94 | 0.24 | 20.43 | 0.20 | 1.63 | 97.60 | 1.33 |
| | MI051 | 1.91 | 2.44 | 78.28 | 2.73 | 5.20 | 0.67 | 0.21 | 7.71 | 0.05 | 2.76 | 90.05 | 0.67 |
| | MI053 | 3.22 | 4.06 | 79.31 | 1.39 | 2.33 | 0.56 | 0.17 | 11.87 | 0.07 | 2.38 | 83.33 | 0.97 |
| G3: | MI031 | 20.80 | 30.40 | 68.42 | 2.91 | 12.27 | 8.85 | 0.41 | 13.94 | 1.25 | 2.67 | 99.81 | 8.12 |
| anti-hPD-1 | MI041 | 23.00 | 35.10 | 65.53 | 1.08 | 6.34 | 3.79 | 0.32 | 29.77 | 1.12 | 1.13 | 100.00 | 3.97 |
| | MI055 | 6.17 | 9.53 | 64.74 | 3.01 | 12.96 | 2.87 | 0.44 | 14.48 | 0.42 | 2.36 | 87.08 | 2.25 |
| | MI058 | 7.72 | 12.90 | 59.84 | 1.93 | 9.57 | 2.49 | 0.32 | 16.58 | 0.41 | 1.46 | 98.31 | 1.88 |
| | MI060 | 4.45 | 6.79 | 65.54 | 4.76 | 12.69 | 3.23 | 0.79 | 16.61 | 0.54 | 1.78 | 64.90 | 1.21 |
| G4: | MI037 | 8.66 | 12.20 | 70.98 | 1.67 | 9.51 | 2.04 | 0.44 | 26.13 | 0.54 | 2.28 | 99.56 | 2.78 |
| anti-hPD-L1 | MI046 | 15.00 | 22.00 | 68.18 | 2.61 | 10.84 | 5.74 | 0.80 | 30.65 | 1.76 | 2.28 | 97.23 | 5.02 |
| | MI048 | 11.60 | 17.70 | 65.54 | 1.42 | 12.49 | 2.51 | 0.62 | 43.46 | 1.10 | 2.10 | 88.77 | 3.72 |
| | MI062 | 3.28 | 7.22 | 45.43 | 1.80 | 8.78 | 1.30 | 0.48 | 26.39 | 0.35 | 1.78 | 91.52 | 1.29 |
| G5: | MI034 | 2.00 | 2.90 | 68.97 | 3.12 | 6.58 | 0.90 | 0.40 | 12.82 | 0.12 | 2.46 | 65.29 | 0.71 |
| OBI-3424 + | MI042 | 0.24 | 0.49 | 48.28 | 5.87 | 10.28 | 0.29 | 0.80 | 13.54 | 0.04 | 2.83 | 63.38 | 0.14 |
| anti-hPD-1 | MI050 | 1.71 | 2.91 | 58.76 | 3.98 | 12.46 | 1.16 | 0.82 | 20.50 | 0.24 | 2.11 | 68.62 | 0.61 |
| | MI054 | 0.89 | 1.21 | 73.88 | 4.04 | 8.60 | 0.49 | 0.59 | 14.60 | 0.07 | 1.99 | 70.82 | 0.24 |
| | MI056 | 2.06 | 3.13 | 65.81 | 3.66 | 7.72 | 1.15 | 0.43 | 11.76 | 0.13 | 1.99 | 62.09 | 0.62 |
| G6: | MI033 | 2.16 | 3.20 | 67.50 | 1.71 | 4.79 | 0.55 | 0.39 | 22.81 | 0.12 | 2.57 | 85.50 | 0.82 |
| OBI-3424 + | MI038 | 4.46 | 5.69 | 78.38 | 3.94 | 7.12 | 2.24 | 0.77 | 19.42 | 0.44 | 4.00 | 90.69 | 2.28 |
| antihPD-L1 | MI043 | 3.20 | 4.50 | 71.11 | 3.32 | 11.18 | 1.49 | 0.40 | 11.90 | 0.18 | 4.01 | 67.25 | 1.80 |
| | MI044 | 1.06 | 1.81 | 58.56 | 3.01 | 8.98 | 0.54 | 0.46 | 15.28 | 0.08 | 2.79 | 85.43 | 0.50 |
| | MI045 | 3.28 | 4.20 | 78.10 | 3.09 | 6.87 | 1.30 | 0.71 | 23.01 | 0.30 | 4.69 | 90.36 | 1.97 |

CD45⁺ Gated % = Gate CD45⁺ cells
Cell count (×10⁴) = Total cell × Total % × 10
NK Gated % = Gate CD45⁺CD56⁺ cells

TABLE 7
Summary for percentage of PD-L1 cells in TILs

| | | | | | | | | | | | Total PD-L1⁺ cells | | |
|---|---|---|---|---|---|---|---|---|---|---|---|---|---|
| | | | | | | | | | | | (PD-L1⁺) | | |
| | | Cell | | | CD45⁺ cells | | | Total cells | | | | Total | |
| | | Conc. | Total | | | | Cell | | | Cell | | cells | Cell |
| | Animal | ×(10⁵/ | cell | Viability | Total | Gated | count | Total | Gated | count | Total | Gated | count |
| Groups | No. | mL) | ×10⁶ | % | % | % | (×10⁶) | % | % | (×10⁶) | % | % | (×10⁵) |
| G1: Vehicle | MI039 | 5.87 | 10.50 | 55.90 | 17.13 | 30.98 | 1.80 | 53.73 | 53.73 | 5.64 | 3.80 | 7.06 | 3.99 |
| | MI047 | 8.63 | 13.10 | 65.88 | 26.08 | 37.00 | 3.42 | 82.52 | 82.52 | 10.81 | 7.38 | 8.94 | 9.67 |
| | MI049 | 5.16 | 12.30 | 41.95 | 12.75 | 18.11 | 1.57 | 81.77 | 81.77 | 10.06 | 24.44 | 29.89 | 30.06 |
| | MI057 | 5.53 | 9.62 | 57.48 | 21.22 | 39.14 | 2.04 | 46.07 | 46.07 | 4.43 | 0.70 | 1.51 | 0.67 |
| | MI059 | 12.30 | 19.00 | 64.74 | 37.05 | 53.59 | 7.04 | 78.60 | 78.60 | 14.93 | 3.86 | 4.90 | 7.33 |
| G2: OBI- | MI035 | 2.85 | 4.36 | 65.37 | 42.64 | 58.88 | 1.86 | 87.63 | 87.63 | 3.82 | 4.97 | 5.67 | 2.17 |
| 3424 | MI036 | 2.81 | 7.37 | 38.13 | 18.14 | 28.16 | 1.34 | 79.89 | 79.89 | 5.89 | 19.30 | 24.15 | 14.22 |
| | MI040 | 5.72 | 8.18 | 69.93 | 29.31 | 40.69 | 2.40 | 84.57 | 84.57 | 6.92 | 5.09 | 6.02 | 4.16 |
| | MI051 | 1.91 | 2.44 | 78.28 | 61.46 | 79.75 | 1.50 | 94.06 | 94.06 | 2.30 | 1.08 | 1.15 | 0.26 |
| | MI053 | 3.22 | 4.06 | 79.31 | 62.53 | 79.11 | 2.54 | 94.44 | 94.44 | 3.83 | 0.62 | 0.65 | 0.25 |
| G3: anti- | MI031 | 20.80 | 30.40 | 68.42 | 25.75 | 41.15 | 7.83 | 53.84 | 53.84 | 16.37 | 3.47 | 6.45 | 10.55 |
| hPD-1 | MI041 | 23.00 | 35.10 | 65.53 | 17.89 | 30.42 | 6.28 | 51.71 | 51.71 | 18.15 | 3.89 | 7.52 | 13.65 |
| | MI055 | 6.17 | 9.53 | 64.74 | 28.22 | 47.95 | 2.69 | 67.88 | 67.88 | 6.47 | 2.50 | 3.68 | 2.38 |
| | MI058 | 7.72 | 12.90 | 59.84 | 20.74 | 36.87 | 2.68 | 77.26 | 77.26 | 9.97 | 6.05 | 7.83 | 7.80 |
| | MI060 | 4.45 | 6.79 | 65.54 | 44.68 | 63.31 | 3.03 | 89.23 | 89.23 | 6.06 | 6.21 | 6.95 | 4.22 |
| G4: | MI037 | 8.66 | 12.20 | 70.98 | 24.35 | 34.93 | 2.97 | 77.18 | 77.18 | 9.42 | 1.11 | 1.43 | 1.35 |
| | MI046 | 15.00 | 22.00 | 68.18 | 32.38 | 47.08 | 7.12 | 75.98 | 75.98 | 16.72 | 1.81 | 2.38 | 3.98 |
| anti-hPD-L1 | MI048 | 11.60 | 17.70 | 65.54 | 20.95 | 31.41 | 3.71 | 74.62 | 74.62 | 13.21 | 8.80 | 11.79 | 15.58 |
| | MI062 | 3.28 | 7.22 | 45.43 | 22.77 | 28.59 | 1.64 | 88.63 | 88.63 | 6.40 | 16.86 | 19.02 | 12.17 |
| G5: | MI034 | 2.00 | 2.90 | 68.97 | 53.60 | 70.26 | 1.55 | 88.56 | 88.56 | 2.57 | 0.85 | 0.96 | 0.25 |
| | MI042 | 0.24 | 0.49 | 48.28 | 54.30 | 81.64 | 0.27 | 53.73 | 53.73 | 0.26 | 1.46 | 2.72 | 0.07 |
| OBI-3424 + | MI050 | 1.71 | 2.91 | 58.76 | 34.76 | 53.31 | 1.01 | 50.19 | 50.19 | 1.46 | 2.12 | 4.21 | 0.62 |
| anti-hPD-1 | MI054 | 0.89 | 1.21 | 73.88 | 49.86 | 70.32 | 0.60 | 49.84 | 49.84 | 0.60 | 1.86 | 3.73 | 0.23 |
| | MI056 | 2.06 | 3.13 | 65.81 | 54.48 | 78.32 | 1.71 | 90.68 | 90.68 | 2.84 | 2.12 | 2.34 | 0.66 |
| G6: | MI033 | 2.16 | 3.20 | 67.50 | 42.38 | 60.88 | 1.36 | 72.79 | 72.79 | 2.33 | 1.67 | 2.29 | 0.53 |
| OBI-3424 + | MI038 | 4.46 | 5.69 | 78.38 | 64.51 | 81.40 | 3.67 | 80.45 | 80.45 | 4.58 | 0.74 | 0.92 | 0.42 |
| anti-hPD-L1 | MI043 | 3.20 | 4.50 | 71.11 | 57.46 | 79.35 | 2.59 | 88.15 | 88.15 | 3.97 | 1.48 | 1.67 | 0.67 |
| | MI044 | 1.06 | 1.81 | 58.56 | 42.53 | 64.37 | 0.77 | 89.54 | 89.54 | 1.62 | 2.50 | 2.79 | 0.45 |
| | MI045 | 3.28 | 4.20 | 78.10 | 52.11 | 72.03 | 2.19 | 93.65 | 93.65 | 3.93 | 1.00 | 1.06 | 0.42 |

The results indicated that administration of anti-hPD-1 or anti-hPD-L1 had no effect on tumor growth. In contrast, administration of OBI-3424, combined treatment of OBI-3424 and anti-hPD-1, or combined treatment of OBI-3424 and anti-hPD-L1 demonstrated efficiently anti-tumor effect on tumor growth in HepG2 humanized mouse model.

### Example 2. Efficacy Evaluation of OBI-3424+Anti-PD-1 (Pembrolizumab) in HepG2 Tumor Bearing Humanized Mice Model

The aim of study is to evaluate the efficacy of different doses of test item OBI-3424 on tumor growth in presence of anti-PD-1 antibody (Pembrolizumab) on HepG2 humanized mouse model and the impact of CD8⁺ T cells depletion on the anti-tumor effect of combined treatment (OBI-3424/anti-PD-1).

### Material

1. OBI-3424-DP
Lot number: FLC-INJ-1711-01
Number of test item: 2 vials/1 mL per vial
Ingredient: DNA alkylating agents
Concentration: 10 mg/mL
Physical appearance: clear liquid
Storage condition: −20° C.

2. Anti-Human PD-1, Pembrolizumab, Merck & Co., Inc.
Lot number: 7006846900
Number of test item: 1 vials/100 mg per vial
Ingredient: antibody
Concentration: 25 mg/mL
Physical appearance: clear liquid
Storage condition: 2-8° C.

3. Isotonic Sodium Chloride Solution (Taiwan Biotech Co., LTD)
Lot number: 10P2A092
Concentration: 0.9% Sodium chloride
Physical appearance: clear liquid
Solubility: not provided
Storage condition: Room Temperature

4. Matrigel (Corning, Cat. No.: 354248, Lot No.: 0261002)

5. Human PBMC (Lot: PBMC102219D, Zenbio, USA)

6. Collagenase (Sigma Aldrich, C5138), DNase I (Sigma Aldrich, D5025), Hyaluronidase (Sigma Aldrich, H6254)

7. RBC red blood cell lysis buffer (Biolegend, 420302)

8. Cell staining buffer (Biolegend, 420201)

9. Human TruStain FcX™ (Fc Receptor Blocking Solution) (Biolegend, 422302)

10. Antibodies:

Anti-human CD45 antibody (Biolegend, 368508), anti-human CD8 antibody (Biolegend, 344710), anti-human CD4 antibody (Biolegend, 317429), anti-human CD56 antibody (Biolegend, 318332), antihuman CD11c antibody (Biolegend, 301614), anti-human CD25 antibody (Biolegend, 302610), anti-human CD69 antibody (Biolegend, 310914), anti-human CD86 antibody (Biolegend, 305406), anti-human CD91 antibody (Invitrogen, 46-0919-42), anti-human Foxp3 antibody (Biolegend, 320108), anti-human IFN-7 antibody (Biolegend, 506507), anti-human Granzyme B antibody (Biolegend, 372204), anti-human Calreticulin antibody (Abcam, ab209577), anti-human PD-1 antibody (Biolegend, 329907), antihuman PD-L1 antibody (Biolegend, 329738) and ViaKrome 405 (Biolegend, 302610)

### Mouse

1. Species: Mus musculus
Strain: Advanced immunodeficiency mouse.
(NOD.CgPrkdc^{scid} Il2rg)
Source: Trineo Biotechnology Co., LTD.

Sex: Female
Age at initiation of study: 6-8 weeks
Body weight range at start of study: 17-30 g

2. Numbering and identification: Each mouse was numbered by ear tag. The housing cage was identified by cage card with the information including study number, cage number, animal number, sex, dose level, etc.

Animal grouping: The mice were divided into nine groups: G1 (Vehicle), G2 (OBI-3424 0.3 mg/kg), G3 (OBI-3424 1 mg/kg), G4 (anti-hPD-1), G5 (OBI-3424 0.3 mg/kg+anti-hPD-1), G6 (OBI-3424 1 mg/kg+anti-hPD-1), and G7 (OBI-3424 1 mg/kg+anti-hPD-1, exclude CD8⁺ PBMC). Each group contains six mice. The human hepatocellular carcinoma cell line HepG2 were subcutaneously inoculated to the advanced immune-deficiency mice. Total of 42 mice were included in this study.

3. Reason for animal selection: According to the guidelines for nonclinical studies of anticancer pharmaceuticals in non-clinical safety studies of drug suggestion published by TFDA, animal xenograft tumor models can be used to evaluate the efficacy of new drug or new anticancer drugs. The commonly used mouse strains including BALB/c, C57BL/6, whereas BALB/c Nude, Nu/Nu and NOD/SCID mice are usually selected for evaluating the anti-tumor effect of desired drugs. The strains are managed on a global basis with well-known genetic and breed background, which can provide a valuable insight into functional significance of a proper reaction in human body.

4. Period of acclimatization: The mice were acclimated for at least 3 days before randomization. Clinical observations and body weight measurements was performed during acclimation period. The animals did not show any signs of illness or altered behavior during this period.

5. Animal housing condition: The mice were housed in individually ventilated cages (IVC) with the sterile bedding (10054, Andersons, USA) in a controlled environment with temperature 22±3° C., relative humidity 50±20% and 12/12 hr light/dark cycle. The food (LabDiet 5010, PMI, USA) and water (sterile RO water) were provided ad libitum throughout the whole study period.

6. Randomization: All animals were weighed, and the healthy conditions were observed prior to study. Animals without abnormal clinical signs were selected in the experiment. The healthy animals were randomized into different groups without significant difference in the body weight between groups. The weight variation of the animals should not exceed ±20% of the mean body weight. The procedure followed the standards of laboratory animal practices.

7. IACUC approval number: IACUC-2020-SH-024

### Equipment

Cell culture incubator (Shel Lab/3552)
Biosafety cabinet (BAKER/SG604)
Electronic balance (PRECISA/XS 225A-SCS)
Pipettes (Thermo/Finnpipette F1)
Isolated positive/negative pressure validated cage housing system (Allentown/NEXGEN)
Analytical balance (PRECISA/XS3250C-SCS)
Animal euthanasia equipment (Forward Biotech Supply)
Flow Cytometer (Beckman Coulter/Navios EX)
Vernier (Mitutoyo/CD-6″ ASX)

### Method

### Experimental Design

Sampling: The experimental design, experimental groups, doses and volume of injection, route of administration and animal numbers are listed in Table 8.

TABLE 8
Dosing regimen and sampling

| | Route and | Dose of | Volume of | |
|---|---|---|---|---|
| | administration | injection | injection | Animal |
| Groups | of test item | (mg/kg) | (mg/kg) | No. |
| G1: Vehicle | IV | N/A | 5 | 6 |
| G2: OBI-3424 | IV | 0.3 | 5 | 6 |
| G3: OBI-3424 | IV | 1 | 5 | 6 |
| G4: anti-hPD-1 | IP | 20 | 10 | 6 |
| G5: OBI-3424 + PD-1 | IV and IP | 0.3 + 20 | 5 + 10 | 6 |
| G6: OBI-3424 + PD-1 | IV and IP | 1 + 20 | 5 + 10 | 6 |
| G7: OBI-3424 + PD-1 | IV and IP | 1 + 20 | 5 + 10 | 6 |
| (Exclude CD8⁺ | | | | |
| PBMC) | | | | |

Intravenous injection (IV): Volume of injection is 5 mL/kg
Intraperitoneal injection (IP): Volume of injection is 10 mL/kg

2. Establishment of xenograft mouse model

2.1. Animal hair removal: Prior to the injection of human hepatocellular carcinoma cell line HepG2, the hair on right flank only was removed by clipping.

2.2. Subcutaneous inoculation of tumor cells: 1×10⁷ HepG2 cells were pre-mixed with 0.25×10⁷ hPBMC (cell number ratio 4:1) prior to the mixture of Matrigel (volume ratio 1:1) (Corning, 354248, Lot No.: 0261002). The subcutaneous injection volume was 200 L/mouse.

The day of tumor cell injection was denoted as the first day of latency period (L0).

3. Route and administration of test article:

3.1. The test item OBI-3424 or reference items were intravenously injected to the mice on Day 0. The injection was performed using insulin syringe with the dosage of 0.3 mg/kg or 1 mg/kg and the injection volume was 5 mL/kg. The test item OBI-3424 was continuously administered on Day 7, 14, 21 and 28 for G2, G3, G5, G6 and G7. The reference item was administered for G1. The procedure followed the standards of sample administration. The starting day of test item administration was denoted as the first experimental day (DO).

3.2. The test item anti-hPD-1 antibody were intraperitoneally injected to the mice on Day 2. The injection was performed using insulin syringe with the dosage of 20 mg/kg and injection volume of 10 mL/kg. The test item anti-hPD-1 antibody was continuously administered on Day 5, 9, 12, 16, 19, 23 and 26 for G4, G5, G6, and G7. The procedure followed the standards of sample administration.

3.3. Test item or reference item preparation:

Before administration, the test items were diluted by reference item. The solution concentration of test item OBI-3424 were 0.06 mg/mL and 0.2 mg/mL, and test item anti-hPD-1 was 2 mg/mL.

4. Body weight measurement:

The measurement was performed from the next day of the inoculation. The animal body weights were measured and recorded twice per week.

5. Tumor diameter measurement:

The measurement was performed from the next day of the inoculation. The tumor volumes were measured and recorded twice a week (Mon., Thur.). Tumor volume was calculated by ellipsoid equation according to the records ((major axis×minor axis×minor axis)×(π/6)).

6. Tumor growth inhibition ratio calculation:

Tumor volumes were used to calculate tumor growth inhibition (TGI) rates according to the following formula: TGI (%)=[1−(Ti−T0)/(Ci−C0)]×100, where Ti and Ci indicate the mean tumor volume in the treatment groups and vehicle group at the end of the experiment (Day 30). Whereas, T0 and C0 indicate the mean tumor volumes in the treatment group and vehicle group at the beginning of the experiment (Day 0).

7. Blood sampling:

Submandibular blood sample was collected at end point. At the sacrifice, blood sample could be collected using cardiac puncture. The collected blood sample was centrifuged 15 minutes in 4±2° C. and 1500×g to separate serum and pellet. The upper serum was collected and stored at a temperature below −70° C. The procedure followed the standards of animal blood sampling.

8. Determination of the end point of study:

The study was ended at Day 30.

9. Tumor resection:

Mice were sacrificed by CO₂ euthanasia at the end of the study duration, and the connective tissue around tumor was resected. The tumor samples were then weighed and cut equally into three sections if the tumor weight over 400 mg. The tumor tissue was prepared for the isolation of tumor-infiltrating lymphocytes (TILs). The rest of one section was fixed in 10% formaldehyde and embedded in paraffin; the other was stored at a temperature below −70° C.

10. Isolation of tumor-infiltrating lymphocytes (TILs):

The tumor samples were dissected into smaller fragments using scalpels and then digested with a cocktail of collagenase, DNase I, and Hyaluronidase (Collagenase #C5138, DNase I #D5025, Hyaluronidase #H6254, SigmaAldrich) for at least 2 hours. Tumor digests were then passed through a 70 m mesh cell strainer (Falcon #352350) using an syringe plunger and washed with PBS. Cells were treated with RBC red blood cell lysis buffer (Biolegend #420302), and single-cell suspensions for flow cytometry were prepared.

11. Flow cytometry analysis of TIL populations:

Cells were washed with staining buffer (Biolegend #420201), resuspended in staining buffer containing Fc Receptor Blocking Solution (Biolegend #422302), and incubated for 15 minutes at 4° C. Cells were stained with the fluorescently conjugated surface antibodies and incubated for 30 minutes at 4° C., and then resuspended in staining buffer for flow cytometric analysis. Flow cytometry was done using a Navios EX Flow Cytometer (Beckman Coulter). Data were analyzed using the Kaluza Analysis Software (Beckman Coulter).

12. Statistical analysis:

Results were presented as Mean and standard error of the mean (Mean±SEM). Comparisons of all data collected for each treatment group with concurrent negative control data was calculated using Student's t-test (Microsoft Excel, 2007). P≤0.05 is considered as significance.

### Result

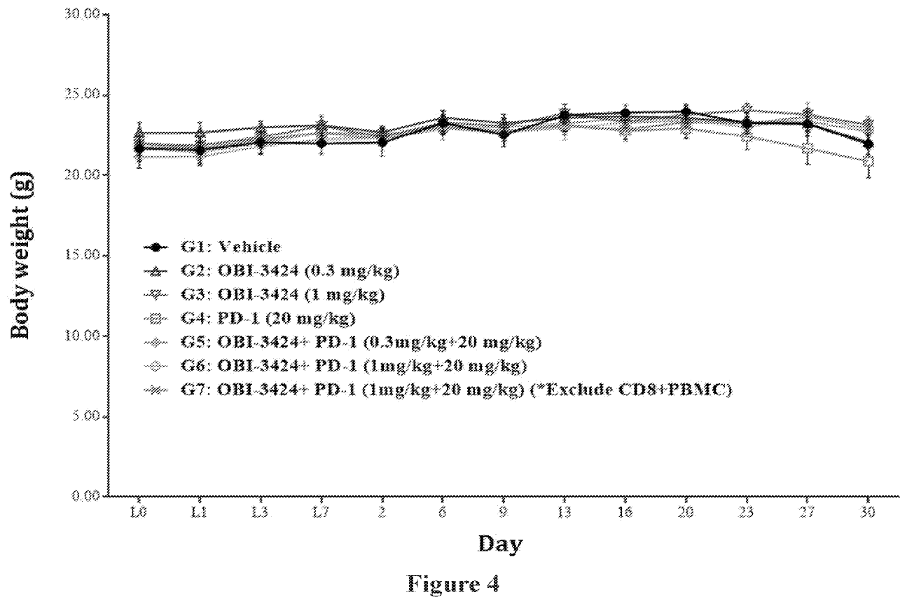

A summary of group body weights was presented in Table 9. There was no statistically significant difference observed in the mean body weight among groups G1-G7 at the beginning of study or at the sacrifice (FIG. 4 and Table 9).

TABLE 9
Summary of body weights

| | Animal | Body Weight (g) | | | | | | | | | | | | |
|---|---|---|---|---|---|---|---|---|---|---|---|---|---|---|
| Groups | No. | L0 | L1 | L3 | L7 | D2 | D6 | D9 | D13 | D16 | D20 | D23 | D27 | D30 |
| G1: Vehicle | MI031 | 22.05 | 21.73 | 22.89 | 22.36 | 23.62 | 25.05 | 24.09 | 24.58 | 24.59 | 24.56 | 24.44 | 24.68 | 22.93 |
| | MI033 | 20.51 | 19.87 | 20.11 | 19.94 | 19.10 | 20.14 | 20.06 | 22.19 | 21.81 | 22.02 | 21.24 | 21.69 | 20.11 |
| | MI054 | 23.71 | 23.62 | 23.44 | 22.98 | 23.28 | 24.10 | 23.93 | 24.23 | 24.65 | 23.87 | 23.06 | 22.54 | 21.47 |
| | MI058 | 24.10 | 24.35 | 24.34 | 24.45 | 24.28 | 24.34 | 24.03 | 24.98 | 25.02 | 25.30 | 25.31 | 25.50 | 24.29 |
| | MI070 | 20.37 | 19.72 | 20.50 | 20.71 | 20.18 | 23.10 | 20.59 | 23.14 | 23.13 | 23.53 | 21.78 | 20.73 | 20.37 |
| | MI079 | 19.28 | 20.19 | 21.02 | 21.52 | 21.78 | 22.78 | 22.54 | 23.33 | 24.20 | 24.51 | 23.71 | 24.04 | 22.57 |
| | Mean | 21.67 | 21.58 | 22.05 | 21.99 | 22.04 | 23.25 | 22.54 | 23.74 | 23.90 | 23.97 | 23.26 | 23.20 | 21.96 |
| | SEM | 0.79 | 0.82 | 0.71 | 0.66 | 0.84 | 0.71 | 0.74 | 0.42 | 0.49 | 0.46 | 0.64 | 0.75 | 0.66 |
| G2: | MI036 | 20.66 | 20.85 | 21.84 | 21.82 | 21.10 | 21.72 | 20.84 | 21.95 | 21.66 | 22.00 | 21.53 | 21.32 | 20.17 |
| OBI-3424 | MI042 | 21.24 | 21.28 | 21.89 | 23.06 | 22.12 | 23.84 | 23.67 | 23.82 | 23.93 | 24.14 | 23.32 | 23.24 | 22.36 |
| (0.3 mg/kg) | MI048 | 23.04 | 22.53 | 23.06 | 23.01 | 22.75 | 23.46 | 22.94 | 23.20 | 23.72 | 23.44 | 23.35 | 23.18 | 21.65 |
| | MI061 | 22.63 | 23.02 | 23.57 | 23.62 | 23.28 | 24.10 | 24.14 | 23.96 | 24.02 | 24.21 | 23.91 | 23.92 | 22.81 |
| | MI090 | 25.00 | 25.12 | 24.12 | 23.73 | 23.23 | 23.68 | 23.38 | 23.82 | 24.56 | 23.98 | 23.98 | 23.80 | 22.44 |
| | MI093 | 23.36 | 23.18 | 23.45 | 23.44 | 23.55 | 24.82 | 24.58 | 24.87 | 24.17 | 23.75 | 23.72 | 23.94 | 22.94 |
| | Mean | 22.66 | 22.66 | 22.99 | 23.11 | 22.67 | 23.60 | 23.26 | 23.60 | 23.68 | 23.59 | 23.30 | 23.23 | 22.06 |
| | SEM | 0.64 | 0.62 | 0.38 | 0.28 | 0.38 | 0.42 | 0.54 | 0.40 | 0.42 | 0.34 | 0.37 | 0.41 | 0.42 |
| G3: | MI038 | 21.11 | 20.59 | 20.70 | 22.50 | 21.69 | 21.91 | 22.01 | 22.55 | 22.68 | 22.87 | 23.02 | 22.66 | 22.31 |
| OBI-3424 | MI039 | 21.33 | 21.16 | 22.24 | 22.44 | 22.40 | 23.15 | 23.65 | 24.14 | 23.51 | 23.39 | 24.10 | 24.41 | 23.72 |
| (1 mg/kg) | MI056 | 23.07 | 22.18 | 22.28 | 22.31 | 22.55 | 23.56 | 22.70 | 23.60 | 23.20 | 24.21 | 23.75 | 23.42 | 23.08 |
| | MI060 | 24.07 | 23.34 | 24.21 | 24.22 | 23.34 | 24.29 | 24.85 | 25.97 | 25.06 | 25.53 | 25.37 | 24.68 | 23.90 |
| | MI077 | 19.46 | 20.56 | 20.80 | 21.42 | 20.20 | 21.84 | 21.52 | 22.22 | 22.25 | 21.98 | 22.89 | 22.84 | 22.02 |
| | MI089 | 22.68 | 23.40 | 24.01 | 25.55 | 24.22 | 24.90 | 23.72 | 24.68 | 23.35 | 24.69 | 25.12 | 24.66 | 23.87 |
| | Mean | 21.95 | 21.87 | 22.37 | 23.07 | 22.40 | 23.28 | 23.08 | 23.86 | 23.34 | 23.78 | 24.04 | 23.78 | 23.15 |
| | SEM | 0.67 | 0.53 | 0.62 | 0.62 | 0.56 | 0.51 | 0.50 | 0.57 | 0.39 | 0.53 | 0.42 | 0.38 | 0.34 |
| G4: PD-1 | MI030 | 21.15 | 22.22 | 22.28 | 22.27 | 22.72 | 23.42 | 23.27 | 23.91 | 24.02 | 23.53 | 24.14 | 23.57 | 22.68 |
| (20 mg/kg) | MI035 | 21.19 | 20.61 | 21.38 | 21.44 | 21.11 | 21.38 | 21.52 | 21.90 | 21.33 | 22.00 | 21.15 | 19.29 | 18.42 |
| | MI045 | 21.82 | 21.84 | 22.87 | 23.16 | 22.69 | 23.51 | 23.58 | 23.56 | 24.01 | 24.34 | 23.47 | 23.24 | 22.49 |
| | MI047 | 21.03 | 20.64 | 21.14 | 22.30 | 21.97 | 22.44 | 21.24 | 21.85 | 21.00 | 20.52 | 19.43 | 19.43 | 18.28 |
| | MI053 | 22.27 | 22.00 | 22.64 | 22.76 | 22.88 | 23.38 | 23.19 | 23.60 | 23.33 | 24.43 | 24.46 | 24.74 | 24.00 |
| | MI087 | 23.11 | 22.72 | 23.21 | 24.04 | 23.93 | 24.02 | 23.71 | 24.09 | 23.04 | 22.69 | 21.87 | 19.83 | 19.38 |
| | Mean | 21.76 | 21.67 | 22.25 | 22.66 | 22.55 | 23.03 | 22.75 | 23.15 | 22.79 | 22.92 | 22.42 | 21.68 | 20.88 |
| | SEM | 0.33 | 0.35 | 0.34 | 0.36 | 0.39 | 0.39 | 0.44 | 0.41 | 0.54 | 0.61 | 0.80 | 0.99 | 1.01 |
| G5: OBI- | MI029 | 20.81 | 21.05 | 21.58 | 21.21 | 21.99 | 22.57 | 22.26 | 22.94 | 23.38 | 23.34 | 22.84 | 22.12 | 21.45 |
| 3424 + PD-1 | MI032 | 20.96 | 21.45 | 22.27 | 22.62 | 23.34 | 23.63 | 23.65 | 23.46 | 24.27 | 24.53 | 24.34 | 24.51 | 23.89 |
| (0.3 mg/kg + | MI049 | 23.40 | 22.90 | 23.13 | 23.49 | 23.00 | 24.07 | 24.30 | 24.48 | 24.34 | 24.57 | 24.17 | 25.20 | 25.43 |
| 20 mg/kg) | MI062 | 22.36 | 21.91 | 22.25 | 23.49 | 23.07 | 23.28 | 23.39 | 23.33 | 23.16 | 23.38 | 23.24 | 23.21 | 22.79 |
| | MI064 | 21.01 | 20.89 | 21.73 | 21.74 | 21.87 | 22.91 | 23.70 | 23.88 | 24.66 | 23.54 | 24.37 | 23.81 | 22.50 |
| | MI076 | 18.35 | 18.83 | 19.93 | 20.92 | 20.68 | 21.15 | 20.75 | 21.24 | 21.70 | 21.41 | 21.08 | 21.23 | 20.31 |
| | Mean | 21.15 | 21.17 | 21.82 | 22.25 | 22.33 | 22.94 | 23.01 | 23.22 | 23.59 | 23.46 | 23.34 | 23.35 | 22.73 |
| | SEM | 0.70 | 0.55 | 0.44 | 0.46 | 0.41 | 0.42 | 0.53 | 0.45 | 0.45 | 0.47 | 0.52 | 0.61 | 0.73 |
| G6: OBI- | MI034 | 20.15 | 19.74 | 20.75 | 21.61 | 21.05 | 21.96 | 21.26 | 21.82 | 23.03 | 22.78 | 21.12 | 21.90 | 21.32 |
| 3424 + PD-1 | MI041 | 21.74 | 21.58 | 23.24 | 23.66 | 23.68 | 24.37 | 24.27 | 24.74 | 24.45 | 25.08 | 24.09 | 25.08 | 24.33 |
| (1 mg/kg + | MI046 | 20.48 | 19.99 | 21.39 | 22.05 | 21.59 | 22.18 | 21.14 | 22.18 | 22.59 | 22.68 | 22.49 | 23.38 | 22.13 |
| 20 mg/kg) | MI059 | 21.61 | 21.78 | 22.69 | 22.56 | 22.69 | 22.46 | 22.64 | 23.04 | 23.84 | 23.62 | 23.58 | 23.76 | 23.73 |
| | MI088 | 22.82 | 22.81 | 21.54 | 22.64 | 22.44 | 23.25 | 23.03 | 23.26 | 22.89 | 23.39 | 23.74 | 24.35 | 23.76 |
| | MI095 | 23.26 | 22.67 | 22.61 | 23.43 | 23.01 | 24.34 | 23.94 | 22.66 | 22.80 | 22.97 | 23.04 | 23.23 | 22.41 |
| | Mean | 21.68 | 21.43 | 22.04 | 22.66 | 22.41 | 23.09 | 22.71 | 22.95 | 23.27 | 23.42 | 23.01 | 23.62 | 22.95 |
| | SEM | 0.50 | 0.53 | 0.39 | 0.32 | 0.39 | 0.44 | 0.54 | 0.42 | 0.29 | 0.36 | 0.44 | 0.44 | 0.48 |
| G7: OBI- | MI066 | 23.91 | 23.62 | 24.10 | 24.30 | 24.26 | 25.08 | 24.84 | 25.43 | 24.90 | 25.28 | 25.31 | 25.50 | 24.54 |
| 3424 + PD-1 | MI067 | 21.49 | 21.56 | 22.25 | 22.47 | 22.97 | 23.44 | 23.23 | 24.16 | 24.11 | 24.43 | 24.52 | 25.14 | 23.89 |
| (1 mg/kg + | MI068 | 20.43 | 19.82 | 20.13 | 20.79 | 20.59 | 21.24 | 21.40 | 21.21 | 21.20 | 22.13 | 22.40 | 22.23 | 22.02 |
| 20 mg/kg) | MI069 | 21.89 | 21.28 | 22.28 | 23.53 | 22.75 | 23.31 | 22.84 | 23.04 | 22.84 | 23.65 | 23.51 | 23.80 | 23.61 |
| (Exclude | MI078 | 19.10 | 18.53 | 19.45 | 19.76 | 19.41 | 20.48 | 20.25 | 20.27 | 20.13 | 20.12 | 19.83 | 20.15 | 19.32 |
| CD8⁺PBMC) | MI091 | 25.33 | 25.36 | 24.88 | 24.91 | 23.78 | 23.98 | 23.71 | 24.11 | 24.00 | 24.24 | 23.75 | 25.17 | 23.88 |
| | Mean | 22.03 | 21.70 | 22.18 | 22.63 | 22.29 | 22.92 | 22.71 | 23.04 | 22.86 | 23.31 | 23.22 | 23.67 | 22.88 |
| | SEM | 8.93 | 1.01 | 0.87 | 0.83 | 0.77 | 0.71 | 0.67 | 0.80 | 0.76 | 0.77 | $.79 | 0.86 | 0.79 |

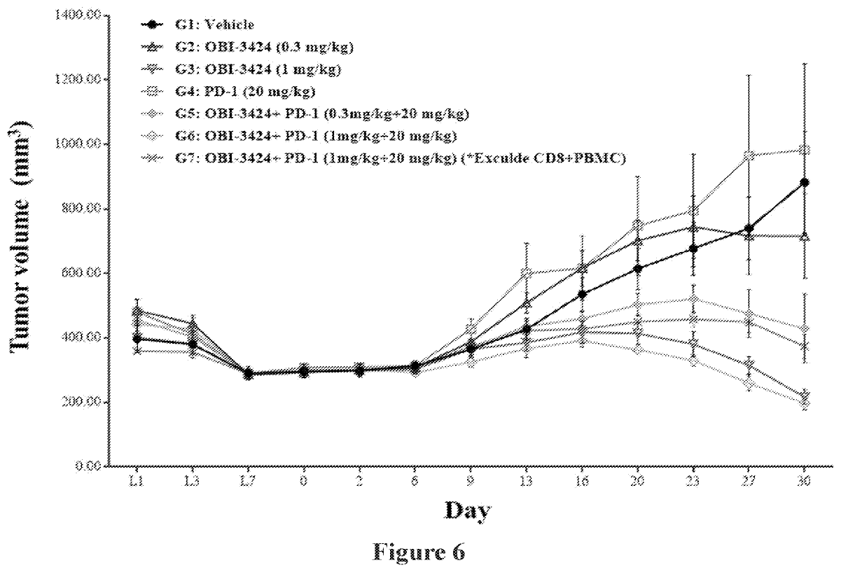

The tumor response was examined from different test items, the mean tumor responses were recorded at L1, 3, and 7 after tumor cell injection and DO, 2, 6, 9, 13, 16, 20, 23, 27, and 30 after test item administration (FIG. 6 and Table 10).

Firstly, we examined the effects of test items from G1 (Vehicle), G2 (OBI-3424 0.3 mg/kg), G3 (OBI-3424 1 mg/kg) and G4 (anti-hPD-1, 20 mg/kg) on tumor response. A dose-related reduction in mean tumor volume was observed in the presence of OBI-3424 (0.3 mg/kg and 1 mg/kg) at D30 (G1 Vehicle: 882.92±158.14 mm³, G2 OBI-3424 0.3 mg/kg: 716.44±31.12 mm³, G3 OBI-3424 1 mg/kg: 216.90±22.20 mm³, p=0.00096<0.001). No statistically significant difference was observed in the treatment of anti-hPD-1 at D30 (G1 Vehicle: 882.92±158.14 mm³, G4 anti-hPD-1: 983.84±266.44 mm³).

With combined treatment of G5 (OBI-3424 0.3 mg/kg+anti-hPD-1 20 mg/kg), G6 (OBI-3424 1 mg/kg+anti-hPD-1 20 mg/kg) and G7 (OBI-3424 1 mg/kg+anti-hPD-1 20 mg/kg, exclude CD8⁺ PBMC), the results showed the mean tumor volume in all of these groups were significantly reduced compared to vehicle group at D30 (G1 Vehicle: 882.92±158.14 mm³, G5 OBI-3424 0.3 mg/kg+anti-hPD-1: 429.41±106.14 mm³, p=0.0193<0.05, G6 OBI-3424 1 mg/kg+anti-hPD-1: 197.74±19.62 mm³, p=0.00078<0.001, G7 OBI-3424 1 mg/kg+anti-hPD-1, exclude CD8⁺ PBMC: 374.44±36.97 mm³, p=0.0053<0.05).

Compared with single treatment, the combined treatment tended to improve the inhibition effect in mean tumor volume at D30 between G2 and G5 and between G3 and G6, however, these reductions were not significant (G2 OBI-3424 0.3 mg/kg: 716.44±31.12 mm³, G5 OBI-3424 0.3 mg/kg+anti-hPD-1: 429.41±106.14 mm³; G3 OBI-3424 1 mg/kg: 216.90±22.20 mm³, G6 OBI-3424 1 mg/kg+anti-hPD-1: 197.74±19.62 mm³). In addition, the percentage of tumor growth inhibition (TGI) was calculated to quantify treatment effects. Single treatment with low- and high-dose OBI-3424 resulted in 27.82% and 113.27% TGI, respectively. Anti-hPD-1 combined treatment with low- and high-dose OBI-3424 resulted in 77.22% and 117.66% TGI, respectively (FIG. 6 and Table 10).

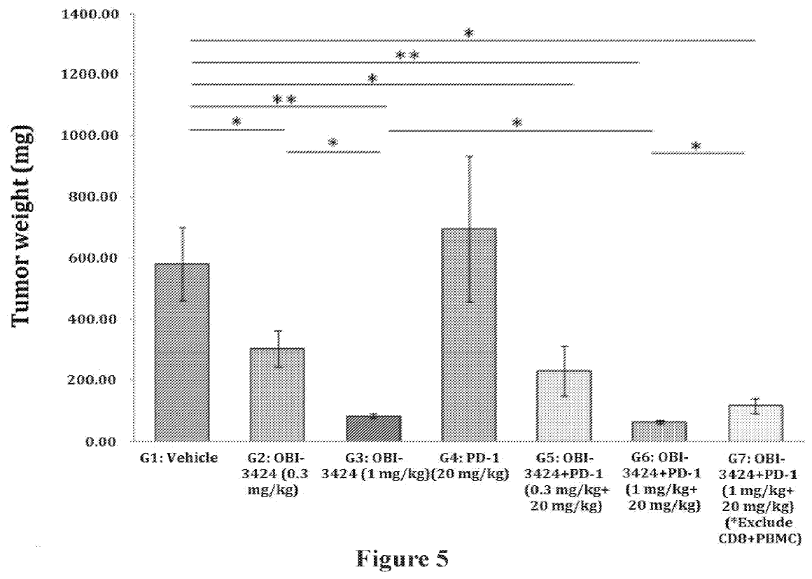

Nevertheless, compared G6 (OBJ-3424 1 mg/kg+anti-hPD-1) with G7 (OBI-3424 1 mg/kg+anti-hPD-1, exclude CD8⁺ PBMC), the depletion of CD8⁺ cells caused a significant increase in mean tumor volume at D30 (G6 OBJ-3424 1 mg/kg+anti-hPD-1: 197.74±19.62 mm³, G7 OBJ-3424 1 mg/kg+anti-hPD-1, exclude CD8⁺ PBMC: 374.44±36.97 mm³, p=0.00088<0.001), and the percentage of TGI was decreased from 117.660% to 87.350% (FIG. 6 and Table 10), despite having the same dose of combined treatment. A similar trend was also observed on the tumor weights (FIG. 5 and Table 10).

TABLE 10
Summary of tumor volumes and weights

| | | | | | | | | | | | | | | | Tumor | |
|---|---|---|---|---|---|---|---|---|---|---|---|---|---|---|---|---|
| | | | | | | | | | | | | | | | weight | |
| | Animal | Tumor volume (mm³) | | | | | | | | | | | | | (mg) | TGI |
| Groups | No. | L1 | L3 | L7 | D0 | D2 | D6 | D9 | D13 | D16 | D20 | D23 | D27 | D30 | D30 | (%) |
| G1: Vehicle | MI031 | 537.1 | 527.4 | 303.8 | 314.2 | 317.2 | 328.7 | 417.9 | 528.2 | 724.3 | 804.6 | 830.3 | 868.4 | 1015. | 615.80 | N/A |
| | MI033 | 398.9 | 395.5 | 278.0 | 285.4 | 286.1 | 287.5 | 320.9 | 335.6 | 424.2 | 529.0 | 543.8 | 563.1 | 673.3 | 486.20 | |
| | MI054 | 352.0 | 347.2 | 276.2 | 277.9 | 279.7 | 281.1 | 390.3 | 520.1 | 569.8 | 641.1 | 817.8 | 841.5 | 867.3 | 805.90 | |
| | MI058 | 485.4 | 463.9 | 368.9 | 373.9 | 375.1 | 378.1 | 403.4 | 418.1 | 421.6 | 422.0 | 422.4 | 422.4 | 423.7 | 91.20 | |
| | MI070 | 251.6 | 251.4 | 215.7 | 217.0 | 222.1 | 292.5 | 317.2 | 407.6 | 632.0 | 789.9 | 916.4 | 1085. | 1562. | 942.50 | |
| | MI079 | 354.0 | 297.0 | 302.9 | 311.8 | 312.9 | 316.9 | 338.8 | 353.9 | 442.6 | 500.7 | 535.2 | 659.7 | 755.3 | 544.80 | |
| | Mean | 396.5 | 380.4 | 290.9 | 296.7 | 298.9 | 314.1 | 364.8 | 427.3 | 535.7 | 614.6 | 677.7 | 740.1 | 882.9 | 581.07 | |
| | SEM | 41.81 | 42.23 | 20.35 | 21.07 | 20.63 | 14.80 | 18.10 | 33.20 | 51.68 | 64.53 | 82.33 | 97.39 | 158.1 | 120.09 | |
| G2: | MI036 | 501.6 | 474.1 | 278.0 | 280.0 | 283.9 | 289.0 | 375.6 | 569.5 | 714.1 | 808.4 | 889.2 | 926.9 | 1012. | 445.50 | 27.82 |
| OBI-3424 | MI042 | 529.3 | 501.4 | 273.7 | 279.4 | 281.1 | 293.9 | 367.0 | 452.6 | 528.0 | 658.4 | 661.3 | 527.6 | 468.2 | 223.90 | |
| (0.3 mg/kg) | MI048 | 346.7 | 337.6 | 216.4 | 226.0 | 230.6 | 241.0 | 395.2 | 528.9 | 714.4 | 854.0 | 890.1 | 909.1 | 882.8 | 327.90 | |
| | MI061 | 442.3 | 401.2 | 336.7 | 353.6 | 354.0 | 356.0 | 367.9 | 388.3 | 405.8 | 419.5 | 297.5 | 205.1 | 181.5 | 63.40 | |
| | MI090 | 597.1 | 491.4 | 313.3 | 317.7 | 321.0 | 330.5 | 388.9 | 590.7 | 749.1 | 751.9 | 842.6 | 848.5 | 850.4 | 449.00 | |
| | MI093 | 493.5 | 461.1 | 301.9 | 303.0 | 334.4 | 336.3 | 437.7 | 524.2 | 591.5 | 721.3 | 884.2 | 886.5 | 903.5 | 317.00 | |
| | Mean | 485.1 | 444.5 | 286.7 | 293.3 | 300.8 | 307.8 | 388.7 | 509.0 | 617.1 | 702.3 | 744.1 | 717.3 | 716.4 | 304.45 | |
| | SEM | 34.55 | 25.76 | 16.95 | 17.53 | 18.24 | 16.99 | 10.83 | 30.95 | 54.63 | 62.98 | 96.27 | 119.0 | 131.1 | 59.47 | |
| G3: | MI038 | 448.8 | 395.2 | 273.6 | 290.8 | 295.5 | 297.0 | 337.5 | 341.3 | 359.9 | 345.0 | 260.7 | 246.4 | 185.2 | 62.40 | 113.27 |
| OBI-3424 | MI039 | 449.7 | 447.9 | 315.2 | 318.0 | 322.4 | 328.4 | 468.5 | 492.3 | 556.8 | 552.1 | 544.3 | 426.0 | 295.5 | 101.90 | |
| (1 mg/kg) | MI056 | 394.4 | 373.2 | 278.1 | 286.8 | 292.9 | 304.4 | 334.0 | 362.5 | 465.2 | 465.2 | 419.2 | 295.9 | 215.0 | 105.20 | |
| | MI060 | 244.8 | 244.1 | 219.0 | 233.0 | 238.8 | 240.6 | 301.7 | 342.7 | 343.3 | 343.7 | 343.7 | 327.7 | 197.1 | 90.30 | |
| | MI077 | 508.7 | 446.7 | 300.7 | 306.6 | 310.3 | 312.8 | 337.3 | 352.9 | 354.8 | 355.2 | 355.2 | 252.6 | 145.5 | 69.80 | |
| | MI089 | 410.4 | 379.8 | 332.7 | 332.7 | 334.6 | 338.1 | 416.4 | 422.6 | 429.4 | 425.6 | 360.3 | 341.1 | 262.8 | 76.80 | |
| | Mean | 409.5 | 381.2 | 286.5 | 294.7 | 299.1 | 303.6 | 365.9 | 385.7 | 418.2 | 414.4 | 380.6 | 315.0 | 216.9 | 84.40 | |
| | SEM | 36.68 | 30.45 | 16.27 | 14.16 | 13.69 | 14.02 | 25.72 | 24.60 | 33.95 | 34.15 | 38.76 | 27.16 | 22.20 | 7.14 | |
| G4: PD-1 | MI030 | 585.9 | 550.0 | 299.8 | 300.3 | 303.5 | 305.3 | 433.1 | 485.3 | 516.7 | 522.8 | 525.5 | 446.8 | 342.6 | 166.60 | −15.29 |
| (20 mg/kg) | MI035 | 508.2 | 411.4 | 273.3 | 277.4 | 280.5 | 284.0 | 469.1 | 934.7 | 982.0 | 1192. | 1346. | 1629. | 1673. | 1230.8 | |
| | MI045 | 554.1 | 364.6 | 280.8 | 283.7 | 285.9 | 288.1 | 339.5 | 382.0 | 382.6 | 391.8 | 416.4 | 600.5 | 674.1 | 453.90 | |
| | M1047 | 400.6 | 393.3 | 332.0 | 345.0 | 346.9 | 348.5 | 552.4 | 762.2 | 772.3 | 1032. | 1076. | 1076. | 1093. | 676.50 | |
| | MI053 | 326.5 | 323.9 | 242.4 | 324.7 | 328.3 | 331.6 | 382.0 | 366.3 | 356.9 | 333.6 | 317.7 | 310.1 | 310.1 | 108.20 | |
| | MI087 | 506.8 | 431.1 | 316.9 | 316.7 | 318.5 | 321.9 | 389.3 | 671.7 | 690.8 | 1015. | 1089. | 1730. | 1808. | 1541.7 | |
| | Mean | 480.4 | 412.4 | 290.9 | 308.0 | 310.6 | 313.2 | 427.6 | 600.4 | 616.9 | 748.1 | 795.2 | 965.6 | 983.8 | 696.28 | |
| | SEM | 40.04 | 31.49 | 13.18 | 10.51 | 10.43 | 10.33 | 30.89 | 92.87 | 99.24 | 152.6 | 174.4 | 249.6 | 266.4 | 237.06 | |
| G5: OBI- | MI029 | 575.5 | 511.0 | 269.1 | 276.3 | 279.9 | 283.4 | 365.0 | 432.8 | 490.6 | 523.4 | 561.4 | 565.7 | 567.2 | 284.00 | 77.22 |
| 3424 + PD-1 | MI032 | 410.1 | 393.6 | 317.4 | 323.5 | 325.6 | 326.8 | 367.2 | 397.3 | 424.8 | 432.7 | 432.7 | 348.6 | 203.6 | 77.30 | |
| (0.3 mg/kg + | MI049 | 527.7 | 452.6 | 331.3 | 338.1 | 345.1 | 348.9 | 424.6 | 433.2 | 437.4 | 441.7 | 442.5 | 314.1 | 237.6 | 87.30 | |
| 20 mg/kg) | MI062 | 392.7 | 318.9 | 242.9 | 248.4 | 252.5 | 254.4 | 283.4 | 386.4 | 390.9 | 426.4 | 430.7 | 399.8 | 305.0 | 149.00 | |
| | MI064 | 364.1 | 346.6 | 286.4 | 295.5 | 298.7 | 301.9 | 373.1 | 469.4 | 475.2 | 555.2 | 573.0 | 427.5 | 372.4 | 173.40 | |
| | MI076 | 464.4 | 397.7 | 298.1 | 293.1 | 296.3 | 297.4 | 399.6 | 500.9 | 539.8 | 640.5 | 690.6 | 799.1 | 890.3 | 609.80 | |
| | Mean | 455.8 | 403.4 | 290.9 | 295.8 | 299.7 | 302.1 | 368.8 | 436.7 | 459.8 | 503.3 | 521.8 | 475.8 | 429.4 | 230.13 | |
| | SEM | 33.69 | 28.57 | 13.15 | 13.15 | 13.37 | 13.48 | 19.49 | 17.60 | 21.65 | 34.93 | 42.90 | 73.72 | 106.1 | 81.77 | |
| G6: OBI- | MI034 | 403.6 | 365.0 | 245.5 | 271.9 | 277.1 | 280.2 | 322.4 | 410.6 | 417.8 | 395.8 | 357.2 | 317.5 | 267.7 | 76.20 | 117.66 |
| 3424 + PD-1 | MI041 | 431.2 | 427.1 | 293.8 | 298.0 | 301.8 | 303.3 | 346.5 | 368.4 | 373.1 | 359.4 | 306.5 | 197.8 | 185.5 | 59.30 | |
| (1 mg/kg + | MI046 | 484.8 | 472.0 | 326.2 | 328.4 | 330.0 | 241.5 | 254.9 | 260.8 | 377.3 | 389.9 | 403.1 | 331.0 | 238.4 | 73.60 | |
| 20 mg/kg) | MI059 | 315.5 | 314.3 | 268.4 | 274.3 | 276.5 | 279.3 | 319.6 | 335.2 | 335.8 | 330.2 | 303.5 | 193.0 | 162.1 | 69.20 | |
| | MI088 | 440.6 | 413.0 | 288.4 | 297.3 | 302.9 | 305.1 | 344.2 | 362.6 | 371.1 | 370.8 | 297.0 | 278.3 | 138.0 | 45.20 | |
| | MI095 | 586.6 | 568.9 | 331.0 | 337.3 | 342.7 | 345.8 | 365.3 | 465.9 | 471.9 | 333.2 | 314.2 | 241.2 | 194.5 | 66.20 | |
| | Mean | 443.7 | 426.7 | 292.2 | 301.2 | 305.2 | 292.5 | 325.5 | 367.3 | 391.2 | 363.2 | 330.3 | 259.8 | 197.7 | 64.95 | |
| | SEM | 36.66 | 36.01 | 13.45 | 11.03 | 11.03 | 14.19 | 15.70 | 28.28 | 19.33 | 11.32 | 17.00 | 24.07 | 19.62 | 4.63 | |
| G7: OBI- | MI066 | 373.0 | 371.9 | 295.3 | 298.1 | 304.0 | 307.0 | 422.4 | 466.3 | 472.2 | 473.1 | 475.1 | 475.6 | 356.3 | 85.30 | 87.35 |
| 3424 + PD-1 | MI067 | 350.1 | 347.3 | 247.0 | 262.5 | 268.3 | 274.2 | 369.0 | 377.4 | 386.5 | 387.4 | 387.4 | 333.1 | 268.0 | 93.60 | |
| (1 mg/kg + | MI068 | 338.4 | 336.6 | 323.7 | 354.0 | 356.0 | 358.0 | 412.1 | 428.2 | 437.2 | 445.7 | 468.4 | 471.5 | 390.8 | 124.20 | |
| 20 mg/kg) | MI069 | 364.3 | 356.6 | 249.3 | 256.3 | 262.9 | 264.5 | 301.9 | 359.9 | 360.6 | 382.0 | 386.1 | 379.4 | 325.8 | 50.60 | |
| (Exclude | M1078 | 387.0 | 382.9 | 293.1 | 304.8 | 309.2 | 311.4 | 363.6 | 457.0 | 458.0 | 524.0 | 534.9 | 535.4 | 537.7 | 255.60 | |
| CD8⁺PBMC) | MI091 | 343.3 | 342.5 | 324.5 | 325.6 | 328.8 | 330.7 | 380.8 | 447.2 | 451.4 | 488.5 | 497.6 | 501.2 | 367.8 | 133.40 | |
| | Mean | 359.4 | 356.3 | 288.8 | 300.2 | 304.8 | 307.6 | 374.9 | 422.7 | 427.6 | 450.1 | 458.3 | 449.4 | 374.4 | 118.78 | |
| | SEM | 7.66 | 7.33 | 13.99 | 15.18 | 14.50 | 14.22 | 17.49 | 17.99 | 18.03 | 23.12 | 24.52 | 31.45 | 36.97 | 24.53 | |

The tumor-infiltrating lymphocytes (TILs) were isolated from fresh tumor tissue, and measured the expression levels of surface markers CD45, CD4, CD8, CD56, CD11c, CD69, CD25, CD86, CD91, Granzyme B, IFN-γ, Foxp3, Calreticulin, PD-1 and PD-L1 among groups by flow cytometer. The numerical data for individual mice were presented in Table 11-16. The population of cytotoxic lymphocytes (CTL) cells (CD45⁺CD8⁺ T cells) and T helper (TH) cells (CD45⁺CD4⁺ T cells) were significantly higher in high-dose OBJ-3424 treatment either alone or combined with anti-hPD-1 compared to vehicle group (CTL cells: G1 Vehicle: 15.53±5.66% G3 OBJ-3424 1 mg/kg: 33.50±3.38%, p=0.0107<0.05, G6 OBI-3424 1 mg/kg+anti-hPD-1: 38.46±2.63%, p=0.00215<0.05; TH cells: G1 Vehicle: 14.59:2.00%, G3 OBI-3424 1 mg/kg: 25.05±2.08%, p=0.0023 K 0.05, G6 OBI-3424 1 mg/kg+anti-hPD-1: 30.62±2.07%, p=0.00012<0.001) (Table 13-14).

TABLE 11
Summary for percentage of Calreticulin cells in TILs

| | | | | | Total CD45⁻ cells | | | CD45⁻ live cells | | | Calreticulin cells | | |
|---|---|---|---|---|---|---|---|---|---|---|---|---|---|
| | | Cell | | | (CD45⁻) | | | (CD45⁻ViaKrome405^{weak}) | | | (CD45⁻ Calreticulin⁺) | | |
| | | Conc. | Total | | | CD45⁺ | Cell | | CTL | Cell | | CTL | Cell |
| | Animal | ×(10⁶/ | cell | Viability | Total | Gated | count | Total | Gated | count | Total | Gated | count |
| Groups | No. | mL) | ×10⁶ | % | % | % | (×10⁵) | % | % | (×10⁵) | % | % | (×10⁵) |
| G1: Vehicle | MI031 | 2.33 | 23.30 | 52.58 | 34.52 | 51.85 | 80.43 | 15.84 | 45.89 | 36.91 | 0.67 | 4.24 | 1.56 |
| | MI033 | 2.60 | 25.95 | 62.24 | 34.54 | 45.88 | 89.63 | 13.47 | 39.00 | 34.95 | 0.87 | 6.44 | 2.26 |
| | MI054 | 2.56 | 25.60 | 64.65 | 30.47 | 41.12 | 78.00 | 15.06 | 49.43 | 38.55 | 0.91 | 6.06 | 2.33 |
| | MI058 | 0.73 | 7.30 | 67.53 | 18.76 | 21.62 | 13.69 | 8.71 | 46.41 | 6.36 | 0.45 | 5.19 | 0.33 |
| | MI070 | 2.43 | 24.30 | 74.90 | 40.29 | 48.60 | 97.90 | 19.59 | 48.62 | 47.60 | 1.11 | 5.68 | 2.70 |
| | MI079 | 4.42 | 44.20 | 72.29 | 28.66 | 36.41 | 126.68 | 16.05 | 56.01 | 70.94 | 1.50 | 9.32 | 6.63 |
| | Mean | 2.51 | 25.11 | 65.70 | 31.21 | 40.91 | 81.06 | 14.79 | 47.56 | 39.22 | 0.92 | 6.16 | 2.63 |
| | SEM | 0.48 | 4.78 | 3.25 | 2.98 | 4.46 | 15.26 | 1.47 | 2.26 | 8.53 | 0.15 | 0.71 | 0.87 |
| G2: | MI036 | 1.95 | 19.45 | 64.78 | 36.29 | 50.59 | 70.58 | 20.24 | 55.78 | 39.37 | 2.85 | 14.09 | 5.54 |
| OBI-3424 | MI042 | 2.05 | 20.45 | 62.35 | 22.44 | 29.66 | 45.89 | 12.36 | 55.07 | 25.28 | 0.62 | 5.02 | 1.27 |
| (0.3 mg/kg) | MI048 | 5.40 | 54.00 | 80.46 | 24.15 | 28.87 | 130.41 | 15.52 | 64.29 | 83.81 | 1.90 | 12.21 | 10.26 |
| | MI061 | 0.21 | 2.14 | 72.34 | 10.20 | 12.41 | 2.18 | 6.30 | 61.79 | 1.35 | 0.28 | 4.51 | 0.06 |
| | MI090 | 2.47 | 24.65 | 80.93 | 26.47 | 31.01 | 65.25 | 12.13 | 45.83 | 29.90 | 0.45 | 3.69 | 1.11 |
| | MI093 | 0.73 | 7.25 | 71.03 | 14.28 | 21.40 | 10.35 | 8.10 | 56.69 | 5.87 | 0.26 | 3.21 | 0.19 |
| | Mean | 2.14 | 21.32 | 71.98 | 22.31 | 28.99 | 54.11 | 12.44 | 56.58 | 30.93 | 1.06 | 7.12 | 3.07 |
| | SEM | 0.74 | 7.41 | 3.15 | 3.77 | 5.18 | 19.06 | 2.06 | 2.61 | 12.12 | 0.44 | 1.94 | 1.66 |
| G3: | MI038 | 0.29 | 2.90 | 68.97 | 8.45 | 9.67 | 2.45 | 5.94 | 70.28 | 1.72 | 0.17 | 2.90 | 0.05 |
| OBI-3424 | MI039 | 1.26 | 12.62 | 80.03 | 12.06 | 13.99 | 15.22 | 7.66 | 63.52 | 9.67 | 0.20 | 2.61 | 0.25 |
| m(1 g/kg) | MI056 | 0.71 | 7.06 | 65.44 | 16.62 | 19.95 | 11.73 | 11.16 | 67.13 | 7.88 | 1.25 | 11.18 | 0.88 |
| | MI060 | 0.99 | 9.88 | 73.48 | 11.79 | 13.15 | 11.65 | 9.18 | 77.84 | 9.07 | 0.25 | 2.75 | 0.25 |
| | MI077 | 0.74 | 7.44 | 64.52 | 11.27 | 12.84 | 8.38 | 7.02 | 62.30 | 5.22 | 0.26 | 3.65 | 0.19 |
| | MI089 | 0.81 | 8.10 | 84.69 | 11.04 | 12.22 | 8.94 | 8.30 | 75.12 | 6.72 | 0.15 | 1.83 | 0.12 |
| | Mean | 0.80 | 8.00 | 72.86 | 11.87 | 13.64 | 9.73 | 8.21 | 69.37 | 6.71 | 0.38 | 4.15 | 0.29 |
| | SEM | 0.13 | 1.32 | 3.32 | 1.09 | 1.40 | 1.76 | 0.74 | 2.55 | 1.19 | 0.17 | 1.43 | 0.12 |
| G4: PD-1 | MI030 | 3.86 | 38.55 | 66.41 | 30.12 | 39.67 | 116.11 | 18.34 | 60.90 | 70.70 | 0.55 | 2.99 | 2.12 |
| (20 mg/kg) | MI035 | 10.32 | 103.20 | 64.44 | 16.86 | 35.97 | 174.00 | 7.84 | 46.50 | 80.91 | 0.29 | 3.72 | 2.99 |
| | MI045 | 2.67 | 26.65 | 63.41 | 44.65 | 67.00 | 118.99 | 27.23 | 60.98 | 72.57 | 0.42 | 1.53 | 1.12 |
| | MI047 | 24.70 | 247.00 | 73.48 | 17.29 | 27.78 | 427.06 | 11.87 | 68.63 | 293.19 | 0.34 | 2.83 | 8.40 |
| | MI053 | 2.84 | 28.35 | 72.84 | 17.20 | 20.43 | 48.76 | 8.94 | 51.99 | 25.34 | 0.17 | 1.88 | 0.48 |
| | MI087 | 7.96 | 79.60 | 44.22 | 13.96 | 28.78 | 111.12 | 4.37 | 31.30 | 34.79 | 0.30 | 6.78 | 2.39 |
| | Mean | 8.73 | 87.23 | 64.13 | 23.35 | 36.61 | 166.01 | 13.10 | 53.38 | 96.25 | 0.35 | 3.29 | 2.92 |
| | SEM | 3.43 | 34.33 | 4.34 | 4.85 | 6.67 | 54.67 | 3.41 | 5.43 | 40.43 | 0.05 | 0.77 | 1.16 |
| G5: OBI- | MI029 | 6.00 | 60.00 | 65.75 | 18.72 | 22.49 | 112.32 | 12.14 | 64.85 | 72.84 | 0.56 | 4.58 | 3.36 |
| 3424 + PD-1 | MI032 | 0.69 | 6.86 | 61.81 | 11.22 | 12.86 | 7.70 | 8.70 | 77.57 | 5.97 | 0.27 | 3.13 | 0.19 |
| (0.3 mg/kg + | MI049 | 0.94 | 9.44 | 59.96 | 10.46 | 11.76 | 9.87 | 7.77 | 74.33 | 7.33 | 0.58 | 7.51 | 0.55 |
| 20 mg/kg) | MI062 | 2.40 | 24.00 | 47.08 | 42.80 | 50.53 | 102.72 | 8.18 | 19.12 | 19.63 | 0.24 | 2.93 | 0.58 |
| | MI064 | 2.52 | 25.20 | 62.22 | 24.16 | 28.02 | 60.88 | 10.99 | 45.49 | 27.69 | 0.80 | 7.31 | 2.02 |
| | MI076 | 3.17 | 31.65 | 47.71 | 35.75 | 52.15 | 113.15 | 18.92 | 52.91 | 59.88 | 0.99 | 5.22 | 3.13 |
| | Mean | 2.62 | 26.19 | 57.42 | 23.85 | 29.64 | 67.77 | 11.12 | 55.71 | 32.23 | 0.57 | 5.11 | 1.64 |
| | SEM | 0.78 | 7.82 | 3.26 | 5.37 | 7.30 | 20.23 | 1.71 | 8.87 | 11.41 | 0.12 | 0.81 | 0.57 |
| G6: OBI- | MI034 | 0.70 | 7.00 | 58.57 | 11.25 | 13.01 | 7.88 | 7.08 | 62.92 | 4.96 | 0.36 | 5.14 | 0.25 |
| 3424 + PD-1 | MI041 | 0.75 | 7.54 | 48.01 | 12.45 | 14.26 | 9.39 | 9.13 | 73.31 | 6.88 | 0.23 | 2.50 | 0.17 |
| (1 mg/kg + | MI046 | 0.95 | 9.50 | 54.32 | 10.29 | 12.12 | 9.78 | 6.32 | 61.43 | 6.00 | 0.26 | 4.05 | 0.25 |
| 20 mg/kg) | MI059 | 0.54 | 5.36 | 48.88 | 10.13 | 11.15 | 5.43 | 7.00 | 69.15 | 3.75 | 0.25 | 3.54 | 0.13 |
| | MI088 | 0.28 | 2.84 | 44.23 | 8.02 | 8.81 | 2.28 | 5.55 | 69.11 | 1.58 | 0.43 | 7.71 | 0.12 |
| | MI095 | 0.74 | 7.38 | 59.08 | 18.72 | 20.88 | 13.82 | 15.58 | 83.21 | 11.50 | 0.17 | 1.08 | 0.13 |
| | Mean | 0.66 | 6.60 | 52.18 | 11.81 | 13.37 | 8.09 | 8.44 | 69.86 | 5.78 | 8.28 | 4.00 | 0.18 |
| | SEM | 0.09 | 0.93 | 2.48 | 1.51 | 1.68 | 1.61 | 1.51 | 3.22 | 1.37 | 0.04 | 0.93 | 0.02 |
| G7: OBI- | MI066 | 1.40 | 14.02 | 51.93 | 24.34 | 27.12 | 34.12 | 19.92 | 81.85 | 27.93 | 1.13 | 5.68 | 1.58 |
| 3424 + PD-1 | MI067 | 1.22 | 12.16 | 59.05 | 8.22 | 9.18 | 10.00 | 6.57 | 79.95 | 7.99 | 0.06 | 0.85 | 0.07 |
| (1 mg/kg + | MI068 | 2.64 | 26.40 | 60.15 | 6.81 | 7.56 | 17.98 | 5.17 | 75.97 | 13.65 | 0.20 | 3.94 | 0.53 |
| 20 mg/kg) | MI069 | 0.47 | 4.72 | 38.31 | 6.71 | 7.57 | 3.17 | 4.82 | 71.75 | 2.28 | 0.15 | 3.16 | 0.07 |
| (Exclude | MI078 | 2.54 | 25.40 | 71.65 | 16.11 | 18.71 | 40.92 | 11.09 | 68.84 | 28.17 | 0.87 | 7.86 | 2.21 |
| CD8⁺PBMC) | MI091 | 1.20 | 11.96 | 75.92 | 17.00 | 19.44 | 20.33 | 14.21 | 83.62 | 17.00 | 0.26 | 1.83 | 0.31 |
| | Mean | 1.58 | 15.78 | 59.50 | 13.20 | 14.93 | 21.09 | 10.30 | 77.00 | 16.17 | 0.45 | 3.89 | 0.80 |
| | SEM | 0.35 | 3.46 | 5.55 | 2.91 | 3.29 | 5.82 | 2.44 | 2.39 | 4.28 | 0.18 | 1.05 | 0.36 |

CD45⁻ Gated % = Gate CD45⁻ cells
Live Gated % = Gate CD45⁻ ViaKrome405^{weak} cells
Cell count (×10⁵) = Total cell × Total % × 10

TABLE 12
Summary for percentage of PD-L1⁺ cells in TILs

| | | | | | | | | Total PD-L1⁺ cells | | | | | |
|---|---|---|---|---|---|---|---|---|---|---|---|---|---|
| | | | | | | | | (PD-L1⁺) | | | PD-L1⁺ cells | | |
| | | Cell | | | Total cells | | | | Total | | (CD45⁻ PD-L1⁺) | | |
| | | Conc. | Total | | | | Cell | | cells | Cell | | CD45⁻ | Cell |
| | Animal | ×(10⁶/ | cell | Viability | Total | Gated | count | Total | Gated | count | Total | Gated | count |
| Groups | No. | mL) | ×10⁶ | % | % | % | (×10⁵) | % | % | (×10⁵) | % | % | (×10⁵) |
| G1: Vehicle | MI031 | 2.33 | 23.30 | 52.58 | 48.90 | 48.90 | 113.94 | 3.28 | 6.70 | 7.64 | 1.97 | 7.65 | 4.59 |
| | MI033 | 2.60 | 25.95 | 62.24 | 67.54 | 67.54 | 175.27 | 3.61 | 5.35 | 9.37 | 1.88 | 6.67 | 4.88 |
| | MI054 | 2.56 | 25.60 | 64.65 | 62.60 | 62.60 | 160.26 | 4.06 | 6.49 | 10.39 | 1.96 | 8.58 | 5.02 |
| | MI058 | 0.73 | 7.30 | 67.53 | 88.24 | 88.24 | 64.42 | 2.07 | 2.35 | 1.51 | 0.56 | 3.81 | 0.41 |
| | MI070 | 2.43 | 24.30 | 74.90 | 78.33 | 78.33 | 190.34 | 3.32 | 4.24 | 8.07 | 1.75 | 5.20 | 4.25 |
| | MI079 | 4.42 | 44.20 | 72.29 | 74.74 | 74.74 | 330.35 | 1.67 | 2.23 | 7.38 | 0.58 | 2.34 | 2.56 |
| | Mean | 2.51 | 25.11 | 65.70 | 70.06 | 70.06 | 172.43 | 3.00 | 4.56 | 7.39 | 1.45 | 5.71 | 3.62 |
| | SEM | 0.48 | 4.78 | 3.25 | 5.57 | 5.57 | 36.74 | 0.38 | 0.80 | 1.27 | 0.28 | 0.97 | 0.74 |
| G2: | MI036 | 1.95 | 19.45 | 64.78 | 62.65 | 62.65 | 121.85 | 7.42 | 11.84 | 14.43 | 4.84 | 15.51 | 9.41 |
| OBI-3424 | MI042 | 2.05 | 20.45 | 62.35 | 74.17 | 74.17 | 151.68 | 2.94 | 3.97 | 6.01 | 1.01 | 5.86 | 2.07 |
| (0.3 mg/kg) | MI048 | 5.40 | 54.00 | 80.46 | 82.83 | 82.83 | 447.28 | 4.56 | 5.50 | 24.62 | 1.98 | 9.64 | 10.69 |
| | MI061 | 0.21 | 2.14 | 72.34 | 93.16 | 93.16 | 19.94 | 1.34 | 1.44 | 0.29 | 0.35 | 4.13 | 0.07 |
| | MI090 | 2.47 | 24.65 | 80.93 | 83.93 | 83.93 | 206.89 | 4.33 | 5.16 | 10.67 | 2.69 | 12.53 | 6.63 |
| | MI093 | 0.73 | 7.25 | 71.03 | 68.08 | 68.08 | 49.36 | 2.15 | 3.16 | 1.56 | 0.84 | 7.23 | 0.61 |
| | Mean | 2.14 | 21.32 | 71.98 | 77.47 | 77.47 | 166.17 | 3.79 | 5.18 | 9.60 | 1.95 | 9.15 | 4.91 |
| | SEM | 0.74 | 7.41 | 3.15 | 4.60 | 4.60 | 62.69 | 0.88 | 1.46 | 3.72 | 0.67 | 1.75 | 1.89 |
| G3: | MI038 | 0.29 | 2.90 | 68.97 | 94.96 | 94.96 | 27.54 | 1.58 | 1.66 | 0.46 | 0.27 | 4.18 | 0.08 |
| OBI-3424 | MI039 | 1.26 | 12.62 | 80.03 | 94.48 | 94.48 | 119.23 | 1.19 | 1.26 | 1.50 | 0.24 | 2.31 | 0.30 |
| (1 mg/kg) | MI056 | 0.71 | 7.06 | 65.44 | 86.71 | 86.71 | 61.22 | 2.88 | 3.32 | 2.03 | 0.58 | 3.76 | 0.41 |
| | MI060 | 0.99 | 9.88 | 73.48 | 95.07 | 95.07 | 93.93 | 1.71 | 1.80 | 1.69 | 0.52 | 4.37 | 0.51 |
| | MI077 | 0.74 | 7.44 | 64.52 | 93.62 | 93.62 | 69.65 | 2.24 | 2.39 | 1.67 | 0.66 | 7.16 | 0.49 |
| | MI089 | 0.81 | 8.10 | 84.69 | 94.29 | 94.29 | 76.37 | 2.86 | 3.03 | 2.32 | 0.58 | 4.63 | 0.47 |
| | Mean | 0.80 | 8.00 | 72.86 | 93.19 | 93.19 | 74.66 | 2.08 | 2.24 | 1.61 | 0.48 | 4.40 | 0.38 |
| | SEM | 0.13 | 1.32 | 3.32 | 1.31 | 1.31 | 12.63 | 0.29 | 0.33 | 0.26 | 0.07 | 0.65 | 0.07 |
| G4: PD-1 | MI030 | 3.86 | 38.55 | 66.41 | 79.18 | 79.18 | 305.24 | 2.73 | 3.45 | 10.52 | 1.07 | 3.76 | 4.12 |
| (20 mg/kg) | MI035 | 10.32 | 103.20 | 64.44 | 37.54 | 37.54 | 387.41 | 2.44 | 6.49 | 25.18 | 1.20 | 8.16 | 12.38 |
| | MI045 | 2.67 | 26.65 | 63.41 | 62.56 | 62.56 | 166.72 | 2.54 | 4.07 | 6.77 | 1.47 | 3.98 | 3.92 |
| | MI047 | 24.70 | 247.00 | 73.48 | 60.73 | 60.73 | 1500.03 | 3.53 | 5.82 | 87.19 | 1.46 | 10.27 | 36.06 |
| | MI053 | 2.84 | 28.35 | 72.84 | 89.83 | 89.83 | 254.67 | 2.21 | 2.46 | 6.27 | 1.10 | 7.27 | 3.12 |
| | MI087 | 7.96 | 79.60 | 44.22 | 17.89 | 17.89 | 142.40 | 1.74 | 9.73 | 13.85 | 0.89 | 13.27 | 7.08 |
| | Mean | 8.73 | 87.23 | 64.13 | 57.96 | 57.96 | 459.41 | 2.53 | 5.34 | 24.96 | 1.20 | 7.79 | 11.12 |
| | SEM | 3.43 | 34.33 | 4.34 | 10.83 | 10.83 | 231.34 | 0.24 | 1.07 | 12.76 | 0.09 | 3.50 | 5.18 |
| G5: OBI- | MI029 | 6.00 | 60.00 | 65.75 | 85.20 | 85.20 | 511.20 | 2.36 | 2.77 | 14.16 | 0.92 | 5.55 | 5.52 |
| 3424 + PD-1 | MI032 | 0.69 | 6.86 | 61.81 | 93.50 | 93.50 | 64.14 | 2.09 | 2.24 | 1.43 | 0.70 | 6.28 | 0.48 |
| (0.3 mg/kg + | MI049 | 0.94 | 9.44 | 59.96 | 93.21 | 93.21 | 87.99 | 2.02 | 2.17 | 1.91 | 0.63 | 6.68 | 0.59 |
| 20 mg/kg) | MI062 | 2.40 | 24.00 | 47.08 | 58.90 | 58.90 | 141.36 | 4.94 | 8.38 | 11.86 | 1.20 | 10.78 | 2.88 |
| | MI064 | 2.52 | 25.20 | 62.22 | 81.27 | 81.27 | 204.80 | 2.74 | 3.37 | 6.90 | 0.86 | 5.35 | 2.17 |
| | MI076 | 3.17 | 31.65 | 47.71 | 61.30 | 61.30 | 194.01 | 3.62 | 5.90 | 11.46 | 1.93 | 6.89 | 6.11 |
| | Mean | 2.62 | 26.19 | 57.42 | 78.90 | 78.90 | 200.58 | 2.96 | 4.14 | 7.95 | 1.04 | 6.92 | 2.96 |
| | SEM | 0.78 | 7.82 | 3.26 | 6.25 | 6.25 | 66.17 | 0.446 | 1.02 | 2.21 | 0.20 | 0.81 | 0.98 |
| G6: OBI- | MI034 | 0.70 | 7.00 | 58.57 | 91.38 | 91.38 | 63.97 | 2.62 | 2.87 | 1.83 | 0.71 | 7.78 | 0.50 |
| 3424 + PD-1 | MI041 | 0.75 | 7.54 | 48.01 | 93.44 | 93.44 | 70.45 | 1.96 | 2.09 | 1.48 | 0.33 | 2.81 | 0.25 |
| (1 mg/kg + | MI046 | 0.95 | 9.50 | 54.32 | 94.93 | 94.93 | 90.18 | 1.88 | 1.98 | 1.79 | 0.50 | 7.52 | 0.48 |
| 20 mg/kg) | MI059 | 0.54 | 5.36 | 48.88 | 94.81 | 94.81 | 50.82 | 2.83 | 2.99 | 1.52 | 0.81 | 7.96 | 0.43 |
| | MI088 | 0.28 | 2.84 | 44.23 | 95.18 | 95.18 | 27.03 | 2.02 | 2.12 | 0.57 | 0.56 | 10.01 | 0.16 |
| | MI095 | 0.74 | 7.38 | 59.08 | 95.84 | 95.84 | 70.73 | 1.35 | 1.41 | 1.00 | 0.42 | 2.43 | 0.31 |
| | Mean | 0.66 | 6.60 | 52.18 | 94.26 | 94.26 | 62.20 | 2.11 | 2.24 | 1.36 | 0.56 | 6.42 | 0.35 |
| | SEM | 0.09 | 0.93 | 2.48 | 0.66 | 0.66 | 8.74 | 8.22 | 0.24 | 0.20 | 0.07 | 1.26 | 0.06 |
| G7: OBI- | MI066 | 1.40 | 14.02 | 51.93 | 93.45 | 93.45 | 131.02 | 3.06 | 3.27 | 4.29 | 0.81 | 3.39 | 1.14 |
| 3424 + PD-1 | MI067 | 1.22 | 12.16 | 59.05 | 95.47 | 95.47 | 116.09 | 1.10 | 1.15 | 1.34 | 0.13 | 1.56 | 0.16 |
| (1 mg/kg + | MI068 | 2.64 | 26.40 | 60.15 | 95.72 | 95.72 | 252.70 | 1.79 | 1.87 | 4.73 | 0.40 | 5.38 | 1.06 |
| 20 mg/kg) | MI069 | 0.47 | 4.72 | 38.31 | 95.30 | 95.30 | 44.98 | 1.41 | 1.48 | 0.67 | 0.28 | 4.39 | 0.13 |
| (Exclude | MI078 | 2.54 | 25.40 | 71.65 | 91.26 | 91.26 | 231.80 | 2.42 | 2.65 | 6.15 | 0.77 | 5.35 | 1.96 |
| CD8⁺PBMC) | MI091 | 1.20 | 11.96 | 75.92 | 93.52 | 93.52 | 111.85 | 1.17 | 1.25 | 1.40 | 0.10 | 0.61 | 0.12 |
| | Mean | 1.58 | 15.78 | 59.50 | 94.12 | 94.12 | 148.07 | 1.83 | 1.95 | 3.09 | 0.42 | 3.45 | 0.76 |
| | SEM | 0.35 | 3.46 | 5.55 | 0.70 | 0.70 | 32.25 | 0.32 | 0.35 | 0.92 | 0.13 | 0.81 | 0.31 |

CD45⁻ Gated % = Gate CD45⁻ cells
Cell count (×10⁵) = Total cell × Total % × 10

TABLE 13
Summary for percentage of CTL cells in TILs

| | | CTL cells | | | PD-1⁺ CTL cells | | | CD69⁺ CTL cells | | | CD25⁺ CTL cells | | | Granzyme B⁺ CTL cells | | | IFN-γ⁺ CTL cells | | |
|---|---|---|---|---|---|---|---|---|---|---|---|---|---|---|---|---|---|---|---|
| | | (CD45⁺CD8⁺) | | | (CD45⁺CD8⁺ PD-1⁺) | | | (CD45⁺ CD8⁺CD69⁺) | | | (CD45⁺ CD8⁺CD25⁺) | | | (CD45⁺ CD8⁺GrB+) | | | (CD45⁺ CD8⁺ IFN-γ⁺) | | |
| | | | CD45⁺ | Cell | | CTL | Cell | | CTL | Cell | | CTL | Cell | | CTL | Cell | | CTL | Cell |
| | Animal | Total | Gated | count | Total | Gated | count | Total | Gated | count | Total | Gated | count | Total | Gated | count | Total | Gated | count |
| Groups | No. | % | % | (×10⁵) | % | % | (×10⁵) | % | % | (×10⁵) | % | % | (×10⁵) | % | % | (×10⁵) | % | % | (×10⁵) |
| G1: Vehicle | MI031 | 5.09 | 33.26 | 11.86 | 4.99 | 97.96 | 11.63 | 3.94 | 60.23 | 9.18 | 1.33 | 20.28 | 3.10 | 6.11 | 91.66 | 14.24 | 0.13 | 1.98 | 0.30 |
| | MI033 | 12.58 | 43.90 | 32.65 | 11.28 | 89.64 | 29.27 | 7.98 | 59.50 | 20.71 | 0.73 | 5.43 | 1.89 | 12.87 | 99.84 | 33.40 | 0.28 | 2.17 | 0.73 |
| | MI054 | 11.35 | 39.92 | 29.06 | 10.31 | 90.87 | 26.39 | 8.02 | 64.46 | 20.53 | 0.66 | 5.31 | 1.69 | 13.11 | 98.26 | 33.56 | 0.30 | 2.28 | 0.77 |
| | MI058 | 42.94 | 75.72 | 31.35 | 26.06 | 60.69 | 19.02 | 29.48 | 78.49 | 21.52 | 0.98 | 2.60 | 0.72 | 34.50 | 88.28 | 25.19 | 0.24 | 0.62 | 0.18 |
| | MI070 | 6.94 | 21.57 | 16.86 | 6.12 | 88.19 | 14.87 | 2.35 | 41.51 | 5.71 | 0.30 | 5.30 | 0.73 | 6.01 | 94.83 | 14.60 | 0.14 | 2.27 | 0.34 |
| | MI079 | 14.27 | 40.45 | 63.07 | 11.14 | 78.05 | 49.24 | 7.84 | 53.18 | 34.65 | 0.51 | 3.45 | 2.25 | 16.20 | 99.14 | 71.60 | 0.06 | 0.37 | 0.27 |
| | Means | 15.53 | 42.47 | 30.81 | 11.65 | 84.23 | 25.07 | 9.94 | 59.56 | 18.72 | 0.75 | 7.06 | 1.73 | 14.80 | 95.34 | 32.10 | 0.19 | 1.62 | 0.43 |
| | SEM | 5.66 | 7.39 | 7.31 | 3.08 | 5.38 | 5.55 | 4.03 | 5.00 | 4.20 | 0.15 | 2.69 | 0.37 | 4.28 | 1.89 | 8.64 | 0.04 | 0.36 | 0.10 |
| G2: | MI036 | 8.21 | 43.92 | 15.97 | 7.87 | 95.86 | 15.31 | 5.21 | 62.46 | 10.13 | 0.98 | 11.79 | 1.91 | 5.97 | 66.16 | 11.61 | 0.18 | 2.04 | 0.35 |
| OBI-3424 | MI042 | 26.79 | 72.25 | 54.79 | 18.38 | 68.60 | 37.59 | 23.86 | 73.01 | 48.79 | 1.90 | 5.81 | 3.89 | 33.78 | 97.44 | 69.08 | 0.23 | 0.66 | 0.47 |
| (0.3 mg/kg) | MI048 | 31.80 | 73.64 | 171.72 | 23.04 | 72.47 | 124.42 | 22.84 | 61.79 | 123.34 | 1.43 | 3.87 | 7.72 | 36.33 | 95.50 | 196.18 | 0.20 | 0.54 | 1.08 |
| | MI061 | 45.54 | 66.97 | 9.75 | 22.62 | 49.67 | 4.84 | 35.19 | 85.14 | 7.53 | 1.62 | 3.93 | 0.35 | 30.78 | 85.88 | 6.59 | 0.13 | 0.36 | 0.03 |
| | MI090 | 26.88 | 60.34 | 66.26 | 25.86 | 96.18 | 63.74 | 17.92 | 68.69 | 44.17 | 1.27 | 4.86 | 3.13 | 28.03 | 99.84 | 69.09 | 0.31 | 1.10 | 0.76 |
| | MI093 | 17.47 | 46.12 | 12.67 | 12.75 | 73.00 | 9.24 | 16.34 | 67.19 | 11.85 | 0.80 | 3.31 | 0.58 | 22.98 | 99.74 | 16.66 | 0.15 | 0.64 | 0.11 |
| | Mean | 26.12 | 60.54 | 55.19 | 18.42 | 75.96 | 42.52 | 20.23 | 69.71 | 40.97 | 1.33 | 5.60 | 2.93 | 26.31 | 90.76 | 61.54 | 0.20 | 0.89 | 0.47 |
| | SEM | 5.18 | 5.27 | 25.24 | 2.82 | 7.24 | 18.66 | 4.04 | 3.52 | 18.05 | 0.17 | 1.29 | 1.11 | 4.49 | 5.35 | 29.31 | 0.03 | 0.25 | 0.16 |
| G3: | MI038 | 43.72 | 66.02 | 12.68 | 25.64 | 58.64 | 7.44 | 35.67 | 80.80 | 10.34 | 1.49 | 3.38 | 0.43 | 40.55 | 90.46 | 11.76 | 0.39 | 0.87 | 0.11 |
| OBI-3424 | MI039 | 31.93 | 53.39 | 40.30 | 20.11 | 62.98 | 25.38 | 25.23 | 70.50 | 31.84 | 1.02 | 2.85 | 1.29 | 31.10 | 99.18 | 39.25 | 0.20 | 0.63 | 0.25 |
| (1 mg/kg) | MI056 | 30.92 | 62.97 | 21.83 | 25.04 | 80.96 | 17.68 | 25.96 | 83.69 | 18.33 | 1.26 | 4.07 | 0.89 | 28.95 | 89.16 | 20.44 | 0.28 | 0.85 | 0.20 |
| | MI060 | 22.88 | 38.03 | 22.61 | 19.75 | 86.30 | 19.51 | 12.76 | 59.32 | 12.61 | 1.57 | 7.29 | 1.55 | 21.85 | 96.01 | 21.59 | 0.14 | 0.63 | 0.1 |
| | MI077 | 43.04 | 65.33 | 32.02 | 31.41 | 72.97 | 23.37 | 32.38 | 76.33 | 24.09 | 2.57 | 6.05 | 1.91 | 42.67 | 95.94 | 31.75 | 0.09 | 0.20 | 0.07 |
| | MI089 | 28.52 | 49.11 | 23.10 | 26.74 | 93.75 | 21.66 | 18.14 | 60.89 | 14.69 | 2.34 | 7.84 | 1.90 | 17.29 | 58.25 | 14.00 | 0.18 | 0.61 | 0.15 |
| | Mean | 33.50 | 55.81 | 25.42 | 24.78 | 75.93 | 19.17 | 25.02 | 71.92 | 18.65 | 1.71 | 5.25 | 1.33 | 30.40 | 88.17 | 23.13 | 0.21 | 0.63 | 0.15 |
| | SEM | 3.38 | 4.52 | 3.89 | 1.79 | 5.56 | 2.60 | 3.49 | 4.16 | 3.29 | 0.23 | 0.80 | 0.24 | 3.78 | 5.72 | 4.31 | 0.04 | 0.09 | 0.03 |
| G4: PD-1 | MI030 | 20.34 | 62.25 | 78.41 | 6.46 | 31.77 | 24.90 | 14.94 | 75.13 | 57.59 | 2.23 | 11.21 | 8.60 | 17.56 | 98.76 | 67.69 | 0.07 | 0.38 | 0.27 |
| (20 mg/kg) | MI035 | 8.15 | 58.12 | 84.11 | 5.62 | 68.92 | 58.00 | 9.40 | 52.94 | 97.01 | 1.12 | 6.33 | 11.56 | 16.76 | 96.54 | 172.96 | 0.10 | 0.55 | 1.03 |
| | MI045 | 7.63 | 43.67 | 20.33 | 6.86 | 89.83 | 18.28 | 5.25 | 68.03 | 13.99 | 1.00 | 13.01 | 2.67 | 7.08 | 87.71 | 18.87 | 0.08 | 0.99 | 0.21 |
| | MI047 | 10.44 | 35.54 | 257.87 | 8.64 | 82.69 | 213.41 | 5.14 | 24.85 | 126.96 | 2.05 | 9.91 | 50.64 | 17.18 | 93.27 | 424.35 | 0.16 | 0.85 | 3.95 |
| | MI053 | 37.52 | 64.31 | 106.37 | 8.16 | 21.75 | 23.13 | 31.34 | 74.74 | 88.85 | 1.66 | 3.96 | 4.71 | 42.68 | 98.96 | 121.00 | 0.11 | 0.25 | 0.31 |
| | MI087 | 3.74 | 50.11 | 29.77 | 2.90 | 77.62 | 23.08 | 6.08 | 54.03 | 48.40 | 0.93 | 8.28 | 7.40 | 7.23 | 64.06 | 57.55 | 0.18 | 1.60 | 1.43 |
| | Means | 14.64 | 52.33 | 96.14 | 6.44 | 62.10 | 60.13 | 12.03 | 58.29 | 72.13 | 1.50 | 8.78 | 14.26 | 18.08 | 89.88 | 143.74 | 0.12 | 0.77 | 1.20 |
| | SEM | 5.11 | 4.61 | 35.06 | 0.84 | 11.59 | 31.22 | 4.15 | 7.78 | 16.39 | 0.23 | 1.35 | 7.38 | 5.31 | 5.44 | 60.25 | 0.02 | 0.20 | 0.59 |
| G5: OBI- | MI029 | 16.57 | 34.68 | 99.42 | 10.62 | 64.12 | 63.72 | 8.26 | 50.84 | 49.56 | 0.88 | 5.42 | 5.28 | 13.91 | 96.69 | 83.46 | 0.05 | 0.36 | 0.30 |
| 3424 + PD-1 | MI032 | 39.77 | 63.46 | 27.28 | 15.81 | 39.76 | 10.85 | 29.46 | 73.79 | 20.21 | 1.32 | 3.32 | 0.91 | 32.47 | 99.23 | 22.27 | 0.06 | 0.18 | 0.04 |
| (0.3 mg/kg + | MI049 | 41.68 | 62.07 | 39.35 | 12.52 | 30.05 | 11.82 | 29.44 | 75.60 | 27.79 | 3.58 | 9.20 | 3.38 | 33.30 | 89.55 | 31.44 | 0.08 | 0.23 | 0.08 |
| 20 mg/kg) | MI062 | 18.13 | 50.51 | 43.51 | 7.14 | 39.41 | 17.14 | 11.02 | 69.86 | 26.45 | 1.98 | 12.55 | 4.75 | 16.71 | 99.78 | 40.10 | 0.10 | 0.60 | 0.24 |
| | MI064 | 16.60 | 36.40 | 41.83 | 7.24 | 43.61 | 18.24 | 9.53 | 55.76 | 24.02 | 1.48 | 8.68 | 3.73 | 15.12 | 99.92 | 38.10 | 0.05 | 0.32 | 0.13 |
| | MI076 | 10.51 | 51.48 | 33.26 | 7.30 | 69.47 | 23.10 | 7.28 | 60.72 | 23.04 | 0.98 | 8.20 | 3.10 | 9.90 | 90.63 | 31.33 | 0.08 | 0.73 | 0.25 |
| | Mean | 23.88 | 49.77 | 47.44 | 10.11 | 47.74 | 24.14 | 15.83 | 64.43 | 28.51 | 1.70 | 7.90 | 3.52 | 20.24 | 95.97 | 41.12 | 0.07 | 0.40 | 0.17 |
| | SEM | 5.44 | 5.00 | 10.68 | 1.46 | 6.33 | 8.12 | 4.34 | 4.15 | 4.35 | 0.41 | 1.31 | 0.62 | 4.11 | 1.92 | 8.85 | 0.01 | 0.09 | 0.04 |
| G6: OBI- | MI034 | 38.17 | 61.22 | 26.72 | 13.21 | 34.60 | 9.25 | 24.82 | 71.33 | 17.37 | 1.94 | 5.56 | 1.36 | 33.88 | 96.68 | 23.72 | 0.13 | 0.38 | 0.09 |
| 3424 + PD-1 | MI041 | 36.57 | 60.58 | 27.57 | 13.06 | 35.70 | 9.85 | 28.01 | 76.61 | 21.12 | 2.87 | 7.85 | 2.16 | 27.28 | 91.46 | 20.57 | 0.15 | 0.51 | 0.11 |
| (1 mg/kg + | MI046 | 47.52 | 71.66 | 45.14 | 5.80 | 12.20 | 5.51 | 38.01 | 79.06 | 36.11 | 2.05 | 4.26 | 1.95 | 48.90 | 99.20 | 46.46 | 0.11 | 0.23 | 0.10 |
| 20 mg/kg) | MI059 | 44.53 | 65.11 | 23.87 | 21.69 | 48.72 | 11.63 | 31.83 | 72.07 | 17.06 | 2.77 | 6.28 | 1.48 | 35.22 | 82.90 | 18.88 | 0.11 | 0.25 | 0.06 |
| | MI088 | 32.19 | 44.50 | 9.14 | 14.83 | 46.07 | 4.21 | 21.54 | 74.67 | 6.12 | 2.88 | 10.00 | 0.82 | 15.89 | 52.69 | 4.51 | 0.09 | 0.29 | 0.03 |
| | MI095 | 31.76 | 53.09 | 23.44 | 16.37 | 51.54 | 12.08 | 14.80 | 46.63 | 10.92 | 1.98 | 6.25 | 1.46 | 29.71 | 94.29 | 21.93 | 0.08 | 0.27 | 0.06 |
| | Mean | 38.46 | 59.36 | 25.98 | 14.16 | 38.14 | 8.75 | 26.50 | 70.06 | 18.12 | 2.42 | 6.70 | 1.54 | 31.81 | 86.20 | 22.68 | 0.11 | 0.32 | 0.08 |
| | SEM | 2.63 | 3.87 | 4.71 | 2.11 | 5.90 | 1.32 | 3.30 | 4.83 | 4.20 | 0.19 | 0.81 | 0.19 | 4.42 | 7.08 | 5.53 | 0.01 | 0.04 | 0.01 |
| G7: OBI- | MI066 | 7.49 | 16.15 | 10.50 | 6.13 | 81.85 | 8.59 | 2.88 | 42.02 | 4.04 | 0.89 | 13.00 | 1.25 | 4.77 | 75.68 | 6.69 | 0.10 | 1.59 | 0.14 |
| 3424 + PD-1 | MI067 | 11.27 | 17.67 | 13.70 | 6.64 | 58.94 | 8.07 | 7.44 | 64.95 | 9.05 | 0.84 | 7.30 | 1.02 | 10.92 | 98.66 | 13.28 | 0.17 | 1.52 | 0.21 |
| (1 mg/kg + | MI068 | 3.79 | 5.74 | 10.01 | 3.22 | 85.02 | 8.50 | 1.32 | 44.96 | 3.48 | 0.38 | 12.94 | 1.00 | 1.68 | 71.87 | 4.44 | 0.12 | 5.32 | 0.32 |
| 20 mg/kg) | MI069 | 13.88 | 20.93 | 6.55 | 11.49 | 82.82 | 5.42 | 4.65 | 36.73 | 2.19 | 0.74 | 5.88 | 0.35 | 2.17 | 17.23 | 1.02 | 0.12 | 0.92 | 0.06 |
| (Exclude | MI078 | 3.20 | 5.84 | 8.13 | 2.49 | 77.88 | 6.32 | 0.87 | 34.72 | 2.21 | 0.45 | 17.92 | 1.14 | 1.59 | 91.47 | 4.04 | 0.15 | 8.76 | 0.38 |
| CD8⁺PBMC) | MI091 | 7.51 | 14.95 | 8.98 | 4.62 | 61.50 | 5.53 | 3.56 | 51.33 | 4.26 | 0.50 | 7.16 | 0.60 | 5.97 | 98.87 | 7.14 | 0.06 | 0.99 | 0.07 |
| | Mean | 7.86 | 13.55 | 9.65 | 5.77 | 74.67 | 7.07 | 3.45 | 45.79 | 4.21 | 0.63 | 10.70 | 0.89 | 4.52 | 75.63 | 6.10 | 0.12 | 3.18 | 0.20 |
| | SEM | 1.70 | 2.59 | 0.99 | 1.32 | 4.68 | 0.61 | 0.98 | 4.54 | 1.03 | 0.09 | 1.91 | 0.14 | 1.48 | 12.58 | 1.69 | 0.02 | 1.30 | 0.05 |

CD45⁻ Gated % = Gate CD45⁻ cells
CTL Gated % = Gate CD45⁺CD8⁺ cells
Cell count (×10⁵) = Total cell × Total % × 10

TABLE 14
Summary for percentage of TH cells in TILs

| | | TH cells | | | PD-1⁺ TH cells | | | CD69⁺ TH cells | | | CD25⁺ TH cells | | | Foxp3⁺ Treg cells | | |
|---|---|---|---|---|---|---|---|---|---|---|---|---|---|---|---|---|
| | | (CD45⁺CD4⁺) | | | (CD45⁺CD4⁺ PD-1⁺) | | | (CD45⁺CD4⁺CD69⁺) | | | (CD45⁺CD4⁺CD25⁺) | | | (CD45⁺CD4⁺CD25⁺Foxp3⁺) | | |
| | | | CD45⁺ | Cell | | TH | Cell | | TH | Cell | | TH | Cell | | TH | Cell |
| | Animal | Total | Gated | count | Total | Gated | count | Total | Gated | count | Total | Gated | count | Total | Gated | count |
| Groups | No. | % | % | (×10⁵) | % | % | (×10⁵) | % | % | (×10⁵) | % | % | (×10⁵) | % | % | (×10⁵) |
| G1: Vehicle | MI031 | 9.47 | 61.88 | 22.07 | 9.32 | 98.44 | 21.72 | 8.27 | 59.31 | 19.27 | 3.46 | 24.81 | 8.06 | 0.05 | 1.50 | 0.12 |
| | MI033 | 11.45 | 39.95 | 29.71 | 11.40 | 99.51 | 29.58 | 6.84 | 46.34 | 17.75 | 1.41 | 9.53 | 3.66 | 0.10 | 7.10 | 0.26 |
| | MI054 | 15.68 | 55.17 | 40.14 | 15.58 | 99.34 | 39.88 | 9.07 | 48.48 | 23.22 | 1.88 | 10.07 | 4.81 | 0.13 | 6.79 | 0.3 |
| | MI058 | 13.00 | 22.91 | 9.49 | 12.26 | 94.31 | 8.95 | 7.88 | 64.02 | 5.75 | 1.42 | 11.50 | 1.04 | 0.08 | 5.37 | 0.06 |
| | MI070 | 23.53 | 73.10 | 57.18 | 23.16 | 98.45 | 56.28 | 11.02 | 54.44 | 26.78 | 1.32 | 6.54 | 3.21 | 0.07 | 5.14 | 0.17 |
| | MI079 | 14.43 | 40.91 | 63.78 | 14.23 | 98.61 | 62.90 | 5.57 | 35.29 | 24.62 | 1.22 | 7.71 | 5.39 | 0.04 | 3.62 | 0.18 |
| | Mean | 14.59 | 48.99 | 37.06 | 14.33 | 98.11 | 36.55 | 8.11 | 51.31 | 19.56 | 1.79 | 11.69 | 4.36 | 0.08 | 4.92 | 0.19 |
| | SEM | 2.00 | 7.33 | 8.50 | 1.98 | 0.78 | 8.42 | 0.76 | 4.18 | 3.08 | 0.35 | 2.72 | 0.96 | 0.01 | 0.85 | 0.04 |
| G2: | MI036 | 11.03 | 59.01 | 21.45 | 10.86 | 98.48 | 21.12 | 7.64 | 63.07 | 14.86 | 2.23 | 18.38 | 4.34 | 0.11 | 5.03 | 0.21 |
| OBI-3424 | MI042 | 9.70 | 26.17 | 19.84 | 7.88 | 81.16 | 16.11 | 6.79 | 71.55 | 13.89 | 1.66 | 17.49 | 3.39 | 0.13 | 7.95 | 0.27 |
| (0.3 mg/kg) | MI048 | 7.52 | 17.42 | 40.61 | 7.14 | 94.89 | 38.56 | 5.20 | 57.32 | 28.08 | 1.65 | 18.21 | 8.91 | 0.08 | 5.08 | 0.43 |
| | MI061 | 22.11 | 32.52 | 4.73 | 20.09 | 90.86 | 4.30 | 15.65 | 75.09 | 3.35 | 2.12 | 10.15 | 0.45 | 0.13 | 6.05 | 0.03 |
| | MI090 | 13.12 | 29.44 | 32.34 | 13.06 | 99.60 | 32.19 | 6.87 | 49.45 | 16.93 | 4.23 | 30.45 | 10.43 | 0.16 | 3.69 | 0.39 |
| | MI093 | 13.82 | 36.50 | 10.02 | 13.65 | 98.76 | 9.90 | 8.60 | 44.32 | 6.24 | 2.84 | 14.61 | 2.06 | 0.11 | 3.81 | 0.08 |
| | Mean | 12.88 | 33.51 | 21.50 | 12.11 | 93.96 | 20.36 | 8.46 | 60.13 | 13.89 | 2.46 | 18.22 | 4.93 | 0.12 | 5.27 | 0.24 |
| | SEM | 2.07 | 5.74 | 5.47 | 1.92 | 2.88 | 5.35 | 1.51 | 4.95 | 3.56 | 0.40 | 2.76 | 1.60 | 0.01 | 0.65 | 0.07 |
| G3: | MI038 | 25.00 | 37.76 | 7.25 | 24.22 | 96.86 | 7.02 | 16.35 | 61.50 | 4.74 | 1.46 | 5.51 | 0.42 | 0.07 | 4.64 | 0.02 |
| OBI-3424 | MI039 | 24.72 | 41.33 | 31.20 | 23.33 | 94.38 | 29.44 | 15.22 | 60.62 | 19.21 | 2.88 | 11.47 | 3.63 | 0.06 | 2.22 | 0.08 |
| (1 mg/kg) | MI056 | 17.44 | 35.50 | 12.31 | 16.66 | 95.57 | 11.76 | 13.01 | 67.91 | 9.19 | 2.69 | 14.05 | 1.90 | 0.10 | 3.57 | 0.07 |
| | MI060 | 24.40 | 40.55 | 24.11 | 24.05 | 98.57 | 23.76 | 11.04 | 45.46 | 10.91 | 5.06 | 20.81 | 5.00 | 0.12 | 2.45 | 0.12 |
| | MI077 | 25.26 | 38.34 | 18.79 | 24.52 | 97.07 | 18.24 | 16.92 | 68.87 | 12.59 | 4.55 | 18.53 | 3.39 | 0.06 | 1.41 | 0.04 |
| | MI089 | 33.47 | 57.62 | 27.11 | 33.34 | 99.62 | 27.01 | 20.76 | 63.43 | 16.82 | 3.26 | 9.97 | 2.64 | 0.13 | 3.92 | 0.11 |
| | Mean | 25.05 | 41.85 | 20.13 | 24.35 | 97.01 | 19.54 | 15.55 | 61.30 | 12.24 | 3.32 | 13.39 | 2.83 | 0.09 | 3.04 | 0.07 |
| | SEM | 2.08 | 3.27 | 3.72 | 2.17 | 0.78 | 3.61 | 1.37 | 3.45 | 2.14 | 0.50 | 2.13 | 0.64 | 0.01 | 0.46 | 0.01 |
| G4: PD-1 | MI030 | 12.78 | 39.13 | 49.27 | 9.19 | 71.87 | 35.43 | 8.75 | 63.85 | 33.73 | 3.08 | 22.44 | 11.87 | 0.10 | 3.38 | 0.39 |
| (20 mg/kg) | MI035 | 6.23 | 44.45 | 64.29 | 5.61 | 90.05 | 57.90 | 5.80 | 44.44 | 59.86 | 1.30 | 9.95 | 13.42 | 0.07 | 5.23 | 0.72 |
| | MI045 | 11.05 | 63.24 | 29.45 | 9.68 | 87.55 | 25.80 | 6.80 | 60.36 | 18.12 | 2.74 | 24.33 | 7.30 | 0.04 | 1.31 | 0.11 |
| | MI047 | 19.16 | 65.22 | 473.25 | 18.33 | 95.66 | 452.75 | 5.43 | 20.34 | 134.12 | 4.77 | 17.86 | 117.82 | 0.16 | 3.27 | 3.95 |
| | MI053 | 17.34 | 29.71 | 49.16 | 14.42 | 83.20 | 40.88 | 10.05 | 56.98 | 28.49 | 2.21 | 12.52 | 6.27 | 0.08 | 3.80 | 0.23 |
| | MI087 | 4.73 | 63.41 | 37.65 | 4.03 | 85.19 | 32.08 | 9.41 | 60.75 | 74.90 | 1.81 | 11.70 | 14.41 | 0.23 | 12.58 | 1.83 |
| | Mean | 11.88 | 50.86 | 117.18 | 10.21 | 85.59 | 107.47 | 7.71 | 51.12 | 58.20 | 2.65 | 16.47 | 28.51 | 0.11 | 4.93 | 1.20 |
| | SEM | 2.36 | 6.17 | 71.38 | 2.19 | 3.26 | 69.20 | 0.80 | 6.75 | 17.45 | 0.50 | 2.45 | 17.91 | 0.03 | 1.61 | 0.61 |
| G5: OBI- | MI029 | 26.12 | 54.68 | 156.72 | 23.73 | 90.86 | 142.38 | 9.50 | 37.70 | 57.00 | 1.93 | 7.67 | 11.58 | 0.06 | 3.31 | 0.36 |
| 3424 + PD-1 | MI032 | 24.04 | 38.35 | 16.49 | 19.71 | 81.99 | 13.52 | 15.02 | 65.21 | 10.30 | 2.29 | 9.94 | 1.57 | 0.08 | 3.32 | 0.05 |
| (0.3 mg/kg + | MI049 | 24.72 | 36.82 | 23.34 | 19.89 | 80.46 | 18.78 | 15.43 | 67.23 | 14.57 | 4.06 | 17.69 | 3.83 | 0.11 | 2.76 | 0.10 |
| 20 mg/kg) | MI062 | 16.62 | 46.31 | 39.89 | 11.64 | 70.04 | 27.94 | 7.18 | 59.46 | 17.23 | 3.31 | 27.44 | 7.94 | 0.08 | 2.29 | 0.19 |
| | MI064 | 21.51 | 47.16 | 54.21 | 19.37 | 90.05 | 48.81 | 8.30 | 39.40 | 20.92 | 2.68 | 12.74 | 6.75 | 0.04 | 1.34 | 0.10 |
| | MI076 | 12.90 | 63.18 | 40.83 | 9.65 | 74.81 | 30.54 | 12.65 | 73.71 | 40.04 | 3.52 | 20.51 | 11.14 | 0.10 | 2.84 | 0.32 |
| | Mean | 20.99 | 47.75 | 55.24 | 17.33 | 81.37 | 46.99 | 11.35 | 57.12 | 26.68 | 2.97 | 16.00 | 7.14 | 0.08 | 2.64 | 0.19 |
| | SEM | 2.12 | 4.07 | 21.03 | 2.23 | 3.36 | 19.71 | 1.44 | 6.16 | 7.38 | 0.33 | 3.00 | 1.62 | 0.01 | 0.30 | 0.05 |
| G6: OBI- | MI034 | 32.23 | 51.69 | 22.56 | 24.70 | 76.63 | 17.29 | 17.76 | 60.04 | 12.43 | 3.09 | 10.44 | 2.16 | 0.07 | 2.20 | 0.05 |
| 3424 + PD-1 | MI041 | 25.80 | 42.74 | 19.45 | 20.10 | 77.89 | 15.16 | 15.91 | 61.65 | 12.00 | 3.39 | 13.13 | 2.56 | 0.12 | 3.54 | 0.09 |
| (1 mg/kg + | MI046 | 23.78 | 35.87 | 22.59 | 14.56 | 61.24 | 13.83 | 15.23 | 67.57 | 14.47 | 2.81 | 12.46 | 2.67 | 0.08 | 2.99 | 0.08 |
| 20 mg/kg) | MI059 | 31.85 | 46.58 | 17.07 | 26.28 | 82.51 | 14.09 | 20.82 | 68.88 | 11.16 | 4.28 | 14.15 | 2.29 | 0.09 | 2.06 | 0.05 |
| | MI088 | 37.77 | 52.21 | 10.73 | 28.20 | 74.67 | 8.01 | 23.50 | 69.63 | 6.67 | 6.21 | 18.40 | 1.76 | 0.15 | 2.38 | 0.04 |
| | MI095 | 32.31 | 54.02 | 23.84 | 26.32 | 81.47 | 19.42 | 11.71 | 37.95 | 8.64 | 3.13 | 10.14 | 2.31 | 0.10 | 3.32 | 0.07 |
| | Mean | 30.62 | 47.19 | 19.37 | 23.36 | 75.74 | 14.63 | 17.49 | 60.95 | 10.90 | 3.82 | 13.12 | 2.29 | 0.10 | 2.75 | 0.06 |
| | SEM | 2.07 | 2.83 | 2.00 | 2.09 | 3.14 | 1.58 | 1.72 | 4.87 | 1.14 | 0.52 | 1.23 | 0.13 | 0.01 | 0.25 | 0.01 |
| G7: OBI- | MI066 | 32.30 | 69.63 | 45.28 | 27.95 | 86.55 | 39.19 | 16.28 | 49.59 | 22.82 | 4.90 | 14.94 | 6.87 | 0.08 | 1.63 | 0.11 |
| 3424 + PD-1 | MI067 | 50.70 | 79.45 | 61.65 | 39.02 | 76.97 | 47.45 | 32.52 | 61.40 | 39.54 | 3.32 | 6.26 | 4.04 | 0.12 | 3.62 | 0.1 |
| (1 mg/kg + | MI068 | 56.19 | 85.06 | 148.34 | 45.95 | 81.78 | 121.31 | 19.64 | 40.53 | 51.85 | 3.83 | 7.91 | 10.11 | 0.14 | 3.55 | 0.37 |
| 20 mg/kg) | MI069 | 57.47 | 86.67 | 27.13 | 45.08 | 78.43 | 21.28 | 23.64 | 43.70 | 11.16 | 2.62 | 4.84 | 1.24 | 0.11 | 4.13 | 0.05 |
| (Exclude | MI078 | 38.81 | 70.85 | 98.58 | 25.46 | 65.62 | 64.67 | 10.75 | 26.95 | 27.31 | 4.36 | 10.94 | 11.07 | 0.11 | 2.47 | 0.28 |
| CD8⁺PBMC) | MI091 | 34.79 | 69.23 | 41.61 | 25.26 | 72.62 | 30.21 | 21.56 | 52.60 | 25.79 | 2.99 | 7.29 | 3.58 | 0.07 | 2.41 | 0.08 |
| | Meas | 45.04 | 76.82 | 70.43 | 34.79 | 77.00 | 54.02 | 20.73 | 45.80 | 29.74 | 3.67 | 8.70 | 6.15 | 0.11 | 2.97 | 0.17 |
| | SEM | 4.54 | 3.25 | 18.51 | 3.97 | 2.97 | 14.77 | 2.99 | 4.80 | 5.77 | 0.35 | 1.50 | 1.59 | 0.01 | 0.39 | 0.05 |

CD45⁻ Gated % = Gate CD45⁻ cells
TH Gated % = Gate CD45⁺CD4⁺ cells
Cell count (×10⁵) = Total cell × Total % × 10

TABLE 15
Summary for percentage of NK cells in TILs

| | | | | | NK cells | | | PD-1⁺ NK cells | | |
|---|---|---|---|---|---|---|---|---|---|---|
| | | Cell | | | (CD45⁺CD56⁺) | | | (CD45⁺CD56⁺PD-1⁺) | | |
| | | Conc. | Total | | | CD45⁺ | Cell | | NK | Cell |
| | Animal | ×(10⁶/ | cell | Viability | Total | Gated | count | Total | Gated | count |
| Groups | No. | mL) | ×10⁶ | % | % | % | (×10⁵) | % | % | (×10⁵) |
| G1: Vehicle | MI031 | 2.33 | 23.30 | 52.58 | 0.57 | 3.74 | 1.33 | 0.50 | 88.11 | 1.17 |
| | MI033 | 2.60 | 25.95 | 62.24 | 3.32 | 11.57 | 8.62 | 1.64 | 49.46 | 4.26 |
| | MI054 | 2.56 | 25.60 | 64.65 | 1.92 | 6.74 | 4.92 | 1.04 | 54.49 | 2.66 |
| | MI058 | 0.73 | 7.30 | 67.53 | 4.38 | 7.72 | 3.20 | 1.57 | 35.80 | 1.15 |
| | MI070 | 2.43 | 24.30 | 74.90 | 1.33 | 4.13 | 3.23 | 0.77 | 57.83 | 1.87 |
| | MI079 | 4.42 | 44.20 | 72.29 | 2.66 | 7.53 | 11.76 | 1.08 | 40.51 | 4.77 |
| | Mean | 2.51 | 25.11 | 65.70 | 2.36 | 6.91 | 5.53 | 1.10 | 54.37 | 2.65 |
| | SEM | 0.48 | 4.78 | 3.25 | 0.56 | 1.16 | 1.60 | 0.18 | 7.55 | 0.64 |
| G2: | MI036 | 1.95 | 19.45 | 64.78 | 1.18 | 6.33 | 2.30 | 0.85 | 71.96 | 1.65 |
| OBI-3424 | MI042 | 2.05 | 20.45 | 62.35 | 3.26 | 8.78 | 6.67 | 1.08 | 33.17 | 2.21 |
| (0.3 mg/kg) | MI048 | 5.40 | 54.00 | 80.46 | 3.83 | 8.87 | 20.68 | 2.01 | 52.46 | 10.85 |
| | MI061 | 0.21 | 2.14 | 72.34 | 6.83 | 10.05 | 1.46 | 1.44 | 21.08 | 0.31 |
| | MI090 | 2.47 | 24.65 | 80.93 | 1.00 | 2.25 | 2.47 | 0.88 | 87.65 | 2.17 |
| | MI093 | 0.73 | 7.25 | 71.03 | 2.54 | 6.70 | 1.84 | 1.14 | 44.79 | 0.83 |
| | Mean | 2.14 | 21.32 | 71.98 | 3.11 | 7.16 | 5.90 | 1.23 | 51.85 | 3.00 |
| | SEM | 0.74 | 7.41 | 3.15 | 0.87 | 1.14 | 3.06 | 0.18 | 10.05 | 1.60 |
| G3: | MI038 | 0.29 | 2.90 | 68.97 | 2.64 | 3.99 | 0.77 | 0.91 | 34.34 | 0.26 |
| OBI-3424 | MI039 | 1.26 | 12.62 | 80.03 | 3.38 | 5.65 | 4.27 | 1.30 | 38.34 | 1.64 |
| (1 mg/kg) | MI056 | 0.71 | 7.06 | 65.44 | 2.95 | 6.00 | 2.08 | 1.44 | 48.85 | 1.02 |
| | MI060 | 0.99 | 9.88 | 73.48 | 2.44 | 4.05 | 2.41 | 1.79 | 73.40 | 1.77 |
| | MI077 | 0.74 | 7.44 | 64.52 | 2.63 | 3.99 | 1.96 | 1.40 | 53.12 | 1.04 |
| | MI089 | 0.81 | 8.10 | 84.69 | 2.46 | 4.24 | 1.99 | 2.18 | 88.62 | 1.77 |
| | Mean | 0.80 | 8.00 | 72.86 | 2.75 | 4.65 | 2.25 | 1.50 | 56.11 | 1.25 |
| | SEM | 0.13 | 1.32 | 3.32 | 0.14 | 0.35 | 0.46 | 0.16 | 7.94 | 0.24 |
| G4: PD-1 | MI030 | 3.86 | 38.55 | 66.41 | 5.98 | 18.31 | 23.05 | 0.53 | 8.83 | 2.04 |
| (20 mg/kg) | MI035 | 10.32 | 103.20 | 64.44 | 0.66 | 4.68 | 6.81 | 0.40 | 60.98 | 4.13 |
| | MI045 | 2.67 | 26.65 | 63.41 | 0.82 | 4.67 | 2.19 | 0.26 | 32.35 | 0.69 |
| | MI047 | 24.70 | 247.00 | 73.48 | 1.41 | 4.81 | 34.83 | 0.97 | 68.84 | 23.96 |
| | MI053 | 2.84 | 28.35 | 72.84 | 3.60 | 6.18 | 10.21 | 0.70 | 19.53 | 1.98 |
| | MI087 | 7.96 | 79.60 | 44.22 | 0.57 | 7.62 | 4.54 | 0.20 | 34.51 | 1.59 |
| | Mean | 8.73 | 87.23 | 64.13 | 2.37 | 7.71 | 13.60 | 0.51 | 37.51 | 5.73 |
| | SEM | 3.43 | 34.33 | 4.34 | 0.89 | 2.17 | 5.20 | 0.12 | 9.51 | 3.67 |
| G5: OBI- | MI029 | 6.00 | 60.00 | 65.75 | 1.63 | 3.42 | 9.78 | 0.97 | 59.56 | 5.82 |
| 3424 + PD-1 | MI032 | 0.69 | 6.86 | 61.81 | 3.28 | 5.23 | 2.25 | 0.71 | 21.59 | 0.49 |
| (0.3 mg/kg + | MI049 | 0.94 | 9.44 | 59.96 | 6.65 | 9.90 | 6.28 | 0.73 | 10.95 | 0.69 |
| 20 mg/kg) | MI062 | 2.40 | 24.00 | 47.08 | 6.30 | 17.57 | 15.12 | 0.75 | 11.93 | 1.80 |
| | MI064 | 2.52 | 25.20 | 62.22 | 2.82 | 6.19 | 7.11 | 0.81 | 28.75 | 2.04 |
| | MI076 | 3.17 | 31.65 | 47.71 | 1.05 | 5.15 | 3.32 | 0.39 | 36.88 | 1.23 |
| | Mean | 2.62 | 26.19 | $7.42 | 3.62 | 7.91 | 7.31 | 0.73 | 28.28 | 2.01 |
| | SEM | 0.78 | 7.82 | 3.26 | 0.96 | 2.12 | 1.91 | 0.08 | 7.45 | 0.80 |
| G6: OBI- | MI034 | 0.70 | 7.00 | 58.57 | 5.74 | 9.20 | 4.02 | 0.63 | 10.95 | 0.44 |
| 3424 + PD-1 | MI041 | 0.75 | 7.54 | 48.01 | 2.06 | 3.41 | 1.55 | 0.48 | 23.54 | 0.36 |
| (1 mg/kg + | MI046 | 0.95 | 9.50 | 54.32 | 4.30 | 6.49 | 4.09 | 0.30 | 7.06 | 0.29 |
| 20 mg/kg) | MI059 | 0.54 | 5.36 | 48.88 | 3.70 | 5.40 | 1.98 | 1.11 | 30.09 | 0.59 |
| | MI088 | 0.28 | 2.84 | 44.23 | 3.52 | 4.86 | 1.00 | 0.86 | 24.57 | 0.24 |
| | MI095 | 0.74 | 7.38 | 59.08 | 2.35 | 3.93 | 1.73 | 0.43 | 18.37 | 0.32 |
| | Mean | 0.66 | 6.60 | 52.18 | 3.61 | 5.55 | 2.40 | 0.64 | 19.10 | 0.37 |
| | SEM | 0.09 | 0.93 | 2.48 | 0.55 | 0.85 | 0.54 | 0.12 | 3.57 | 0.05 |
| G7: OBI- | MI066 | 1.40 | 14.02 | 51.93 | 3.44 | 7.42 | 4.82 | 0.60 | 17.56 | 0.84 |
| 3424 + PD-1 | MI067 | 1.22 | 12.16 | 59.05 | 5.44 | 8.53 | 6.62 | 0.75 | 13.74 | 0.91 |
| (1 mg/kg + | MI068 | 2.64 | 26.40 | 60.15 | 3.62 | 5.47 | 9.56 | 0.96 | 26.66 | 2.53 |
| 20 mg/kg) | MI069 | 0.47 | 4.72 | 38.31 | 10.72 | 16.16 | 5.06 | 2.25 | 20.98 | 1.06 |
| (Exclude | MI078 | 2.54 | 25.40 | 71.65 | 4.04 | 7.38 | 10.26 | 0.60 | 14.74 | 1.52 |
| CD8⁺PBMC) | MI091 | 1.20 | 11.96 | 75.92 | 8.08 | 16.08 | 9.66 | 0.76 | 9.41 | 0.91 |
| | Mean | 1.58 | 15.78 | 59.50 | 5.89 | 10.17 | 7.66 | 0.99 | 17.18 | 1.30 |
| | SEM | 0.35 | 3.46 | 5.55 | 1.20 | 1.92 | 1.00 | 0.26 | 2.47 | 0.27 |

CD45⁺ Gated % = Gate CD45⁺ cells
NK Gated % = Gate CD45⁺CD56⁺ cells
Cell count (×10⁵) = Total cell × Total % × 10

TABLE 16
Summary for percentage of Dendritic cells in TILs

| | | | | | DC cells | | | CD86⁺ DC cells | | | CD91⁺ DC cells | | |
|---|---|---|---|---|---|---|---|---|---|---|---|---|---|
| | | Cell | | | (CD45⁺CD11c⁺) | | | (CD45⁺CD11c⁺CD86⁺) | | | (CD45⁺CD11c⁺CD91⁺) | | |
| | | Conc. | Total | | | CD45+ | Cell | | CTL | Cell | | CTL | Cell |
| | Animal | ×(10⁶/ | cell | Viability | Total | Gated | count | Total | Gated | count | Total | Gated | count |
| Groups | No. | mL) | ×10⁶ | % | % | % | (×10⁵) | % | % | (×10⁵) | % | % | (×10⁵) |
| G1: Vehicle | MI031 | 2.33 | 23.30 | 52.58 | 5.45 | 26.37 | 12.70 | 2.98 | 54.77 | 6.94 | 0.07 | 1.32 | 0.16 |
| | MI033 | 2.60 | 25.95 | 62.24 | 13.15 | 41.24 | 34.12 | 6.38 | 48.51 | 16.56 | 0.10 | 0.79 | 0.26 |
| | MI054 | 2.56 | 25.60 | 64.65 | 10.61 | 34.69 | 27.16 | 3.87 | 36.50 | 9.91 | 0.14 | 1.28 | 0.36 |
| | MI058 | 0.73 | 7.30 | 67.53 | 11.86 | 20.97 | 8.66 | 5.36 | 45.23 | 3.91 | 0.08 | 0.67 | 0.06 |
| | MI070 | 2.43 | 24.30 | 74.90 | 4.68 | 15.74 | 11.37 | 2.54 | 54.19 | 6.17 | 0.08 | 1.62 | 0.19 |
| | MI079 | 4.42 | 44.20 | 72.29 | 10.37 | 30.75 | 45.84 | 5.20 | 50.10 | 22.98 | 0.04 | 0.39 | 0.18 |
| | Mean | 2.51 | 25.11 | 65.70 | 9.35 | 28.29 | 23.31 | 4.39 | 48.22 | 11.08 | 0.09 | 1.01 | 0.20 |
| | SEM | 0.48 | 4.78 | 3.25 | 1.42 | 3.78 | 6.08 | 0.61 | 2.76 | 2.98 | 0.01 | 0.19 | 0.04 |
| G2: | MI036 | 1.95 | 19.45 | 64.78 | 5.46 | 32.90 | 10.62 | 3.27 | 59.93 | 6.36 | 0.09 | 1.61 | 0.18 |
| OBI-3424 | MI042 | 2.05 | 20.45 | 62.35 | 11.42 | 30.81 | 23.35 | 7.01 | 61.40 | 14.34 | 0.08 | 0.70 | 0.16 |
| (0.3 mg/kg) | MI048 | 5.40 | 54.00 | 80.46 | 19.59 | 44.83 | 105.79 | 5.11 | 26.07 | 27.59 | 0.10 | 0.51 | 0.54 |
| | MI061 | 0.21 | 2.14 | 72.34 | 12.80 | 19.60 | 2.74 | 5.82 | 45.47 | 1.25 | 0.08 | 0.63 | 0.02 |
| | MI090 | 2.47 | 24.65 | 80.93 | 10.32 | 24.30 | 25.44 | 6.34 | 61.42 | 15.63 | 0.09 | 0.85 | 0.22 |
| | MI093 | 0.73 | 7.25 | 71.03 | 12.66 | 36.70 | 9.18 | 6.50 | 51.39 | 4.71 | 0.06 | 0.44 | 0.04 |
| | Mean | 2.14 | 21.32 | 71.98 | 12.04 | 31.52 | 29.52 | 5.68 | 50.95 | 11.65 | 0.08 | 0.79 | 0.19 |
| | SEM | 0.74 | 7.41 | 3.15 | 1.87 | 3.65 | 15.66 | 0.55 | 5.62 | 3.92 | 0.01 | 0.17 | 0.08 |
| G3: | MI038 | 0.29 | 2.90 | 68.97 | 15.16 | 23.54 | 4.40 | 7.68 | 50.65 | 2.23 | 0.14 | 0.90 | 0.04 |
| OBI-3424 | MI039 | 1.26 | 12.62 | 80.03 | 18.68 | 31.09 | 23.57 | 8.39 | 44.92 | 10.59 | 0.08 | 0.41 | 0.10 |
| (1 mg/kg) | MI056 | 0.71 | 7.06 | 65.44 | 19.60 | 41.01 | 13.84 | 7.73 | 39.45 | 5.46 | 0.09 | 0.45 | 0.06 |
| | MI060 | 0.99 | 9.88 | 73.48 | 12.37 | 19.89 | 12.22 | 6.81 | 55.03 | 6.73 | 0.08 | 0.68 | 0.08 |
| | MI077 | 0.74 | 7.44 | 64.52 | 22.41 | 35.18 | 16.67 | 9.38 | 41.86 | 6.98 | 0.06 | 0.29 | 0.04 |
| | MI089 | 0.81 | 8.10 | 84.69 | 17.47 | 28.58 | 14.15 | 8.78 | 50.27 | 7.11 | 0.07 | 0.41 | 0.06 |
| | Mean | 0.80 | 8.00 | 72.86 | 17.62 | 29.88 | 14.14 | 8.13 | 47.03 | 6.52 | 0.09 | 0.52 | 0.06 |
| | SEM | 0.13 | 1.32 | 3.32 | 1.33 | 2.90 | 2.54 | 0.34 | 2.24 | 1.31 | 0.01 | 0.08 | 0.01 |
| G4: PD-1 | MI030 | 3.86 | 38.55 | 66.41 | 9.61 | 32.74 | 37.05 | 3.50 | 36.43 | 13.49 | 0.04 | 0.37 | 0.15 |
| (20 mg/kg) | MI035 | 10.32 | 103.20 | 64.44 | 3.99 | 34.88 | 41.18 | 1.25 | 31.29 | 12.90 | 0.04 | 1.00 | 0.41 |
| | MI045 | 2.67 | 26.65 | 63.41 | 4.92 | 38.28 | 13.11 | 1.99 | 40.37 | 5.30 | 0.02 | 0.49 | 0.05 |
| | MI047 | 24.70 | 247.00 | 73.48 | 5.24 | 25.68 | 129.43 | 2.40 | 45.76 | 59.28 | 0.13 | 2.52 | 3.21 |
| | MI053 | 2.84 | 28.35 | 72.84 | 9.27 | 16.68 | 26.28 | 3.84 | 41.48 | 10.89 | 0.04 | 0.39 | 0.11 |
| | MI087 | 7.96 | 79.60 | 44.22 | 0.78 | 13.45 | 6.21 | 0.44 | 56.41 | 3.50 | 0.04 | 5.64 | 0.32 |
| | Mean | 8.73 | 87.23 | 64.13 | 5.64 | 26.95 | 42.21 | 2.24 | 41.96 | 17.56 | 0.05 | 1.74 | 0.71 |
| | SEM | 3.43 | 34.33 | 4.34 | 1.37 | 4.14 | 18.29 | 0.53 | 3.51 | 8.51 | 0.02 | 0.85 | 0.50 |
| G5: OBI- | MI029 | 6.00 | 60.00 | 65.75 | 10.77 | 23.07 | 64.62 | 6.36 | 59.06 | 38.16 | 0.04 | 0.33 | 0.24 |
| 3424 + PD-1 | MI032 | 0.69 | 6.86 | 61.81 | 10.83 | 16.62 | 7.43 | 6.15 | 56.79 | 4.22 | 0.04 | 0.41 | 0.03 |
| (0.3 mg/kg + | MI049 | 0.94 | 9.44 | 59.96 | 11.79 | 18.07 | 11.13 | 5.30 | 44.96 | 5.00 | 0.03 | 0.24 | 0.03 |
| 20 mg/kg) | MI062 | 2.40 | 24.00 | 47.08 | 9.62 | 30.34 | 23.09 | 4.71 | 48.94 | 11.30 | 0.11 | 1.16 | 0.26 |
| | MI064 | 2.52 | 25.20 | 62.22 | 15.61 | 32.00 | 39.34 | 7.50 | 48.07 | 18.90 | 0.08 | 0.54 | 0.20 |
| | MI076 | 3.17 | 31.65 | 47.71 | 5.96 | 34.00 | 18.86 | 3.20 | 53.62 | 10.13 | 0.08 | 1.34 | 0.25 |
| | Mean | 2.62 | 26.19 | 57.42 | 10.76 | 25.68 | 27.41 | 5.54 | $1.91 | 14.62 | 0.06 | 0.67 | 0.17 |
| | SEM | 0.78 | 7.82 | 3.26 | 1.28 | 3.04 | 8.72 | 0.61 | 2.23 | 5.18 | 0.01 | 0.19 | 0.05 |
| G6: OBI- | MI034 | 0.70 | 7.00 | 58.57 | 10.74 | 18.16 | 7.52 | 5.54 | 51.56 | 3.88 | 0.10 | 0.97 | 0.07 |
| 3424 + PD-1 | MI041 | 0.75 | 7.54 | 48.01 | 8.66 | 15.07 | 6.53 | 4.54 | 52.35 | 3.42 | 0.11 | 1.29 | 0.08 |
| (1 mg/kg + | MI046 | 0.95 | 9.50 | 54.32 | 16.79 | 26.86 | 15.95 | 7.56 | 45.00 | 7.18 | 0.08 | 0.45 | 0.08 |
| 20 mg/kg) | MI059 | 0.54 | 5.36 | 48.88 | 13.35 | 19.71 | 7.16 | 5.70 | 42.69 | 3.06 | 0.07 | 0.54 | 0.04 |
| | MI088 | 0.28 | 2.84 | 44.23 | 15.24 | 21.31 | 4.33 | 8.17 | 53.62 | 2.32 | 0.04 | 0.29 | 0.01 |
| | MI095 | 0.74 | 7.38 | 59.08 | 9.55 | 16.02 | 7.05 | 5.83 | 61.08 | 4.30 | 0.03 | 0.34 | 0.02 |
| | Mean | 0.66 | 6.60 | 52.18 | 12.39 | 19.52 | 8.09 | 6.22 | $1.05 | 4.03 | 0.07 | 0.65 | 0.05 |
| | SEM | 0.09 | 0.93 | 2.48 | 1.33 | 1.74 | 1.64 | 0.56 | 2.68 | 0.69 | 0.01 | 0.16 | 0.01 |
| G7: OBI- | MI066 | 1.40 | 14.02 | 51.93 | 12.46 | 26.87 | 17.47 | 7.73 | 62.04 | 10.84 | 0.09 | 0.74 | 0.13 |
| 3424 + PD-1 | MI067 | 1.22 | 12.16 | 59.05 | 15.61 | 24.15 | 18.98 | 10.54 | 67.54 | 12.82 | 0.08 | 0.49 | 0.10 |
| (1 mg/kg + | MI068 | 2.64 | 26.40 | 60.15 | 10.93 | 16.58 | 28.86 | 8.14 | 74.42 | 21.49 | 0.10 | 0.91 | 0.26 |
| 20 mg/kg) | MI069 | 0.47 | 4.72 | 38.31 | 13.43 | 19.06 | 6.34 | 10.18 | 75.82 | 4.80 | 0.09 | 0.66 | 0.04 |
| (Exclude | MI078 | 2.54 | 25.40 | 71.65 | 10.68 | 19.89 | 27.13 | 6.91 | 64.72 | 17.55 | 0.10 | 0.97 | 0.25 |
| CD8⁺PBMC) | MI091 | 1.20 | 11.96 | 75.92 | 18.70 | 36.18 | 22.37 | 13.58 | 72.58 | 16.24 | 0.08 | 0.43 | 0.10 |
| | Mean | 1.58 | 15.78 | 59.50 | 13.64 | 23.79 | 20.19 | 9.51 | 69.52 | 13.96 | 0.09 | 0.70 | 0.15 |
| | SEM | 0.35 | 3.46 | 5.55 | 1.25 | 2.90 | 3.31 | 1.00 | 2.28 | 2.38 | 0.00 | 0.09 | 0.04 |

CD45⁺ Gated % = Gate CD45⁺ cells
DC Gated % = Gate CD45⁺CD11c⁺ cells
Cell count (×10⁵) = Total cell × Total % × 10

The results indicated that administration of OBJ-3424 suppresses tumor growth to a significantly greater degree and showed a dose-dependent trend with anti-tumor activity. Moreover, high-dose OBJ-3424 combined with anti-hPD-1 treatment displayed the most efficiently anti-tumor effect on tumor growth in HepG2 humanized mouse model. However, depletion of CD8⁺ cells resulted in significantly impaired antitumor efficacy of combined treatment.

The above description of embodiments of the present invention does not limit the present invention. Those skilled in the art can make various modifications and changes according to the present invention, and any modification and change within the spirit of the present invention shall be covered in the scope of the claims appended to the present invention.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

### REFERENCE

1. Lin H K, Jez J M, Schlegel B P, Peehl D M, Pachter J A, Penning T M. Expression and characterization of recombinant type 2 3 alpha-hydroxysteroid dehydrogenase (HSD) from human prostate: demonstration of bifunctional 3 alpha/17 beta-HSD activity and cellular distribution. Mol Endocrinol 1997; 11:1971-84
2. Dufort I, Rheault P, Huang X F, Soucy P, Luu—The V. Characteristics of a highly labile human type 5 17beta-hydroxysteroid dehydrogenase. Endocrinology 1999; 140:568-74
3. Penning T M, Drury J E. Human aldo-keto reductases: Function, gene regulation, and single nucleotide polymorphisms. Arch Biochem Biophys 2007; 464:241-50
4. Rizner T L, Penning T M. Role of aldo-keto reductase family 1 (AKR1) enzymes in human steroid metabolism. Steroids 2014; 79:49-63
5. Chang T S, Lin H K, Rogers K A, Brame L S, Yeh M M, Yang Q, et al. Expression of aldo-keto reductase family 1 member C3 (AKR1C3) in neuroendocrine tumors & adenocarcinomas of pancreas, gastrointestinal tract, and lung. Int J Clin Exp Pathol 2013; 6:2419-29
6. Guise C P, Abbattista M R, Singleton R S, Holford S D, Connolly J, Dachs G U, et al. The bioreductive prodrug PR-104A is activated under aerobic conditions by human aldo-keto reductase 1C3. Cancer Res 2010; 70:1573-84
7. Miller V L, Lin H K, Murugan P, Fan M, Penning T M, Brame L S, et al. Aldo-keto reductase family 1 member C3 (AKR1C3) is expressed in adenocarcinoma and squamous cell carcinoma but not small cell carcinoma. Int J Clin Exp Pathol 2012; 5:278-89
8. Zhao J, Zhang M, Liu J, Liu Z, Shen P, Nie L, et al. AKR1C3 expression in primary lesion rebiopsy at the time of metastatic castration-resistant prostate cancer is strongly associated with poor efficacy of abiraterone as a first-line therapy. Prostate 2019; 79:1553-62
9. Nakarai C, Osawa K, Akiyama M, Matsubara N, Ikeuchi H, Yamano T, et al. Expression of AKR1C3 and CNN3 as markers for detection of lymph node metastases in colorectal cancer. Clin Exp Med 2015; 15:333-41
10. Hsu N Y, Ho H C, Chow K C, Lin T Y, Shih C S, Wang L S, et al. Overexpression of dihydrodiol dehydrogenase as a prognostic marker of non-small cell lung cancer. Cancer Res 2001; 61:2727-31
11. Powell K, Semaan L, Conley-LaComb M K, Asangani I, Wu Y M, Ginsburg K B, et al. ERG/AKR1C3/AR Constitutes a Feed-Forward Loop for AR Signaling in Prostate Cancer Cells. Clin Cancer Res 2015; 21:2569-79
12. Heibein A D, Guo B, Sprowl J A, Maclean D A, Parissenti A M. Role of aldo-keto reductases and other doxorubicin pharmacokinetic genes in doxorubicin resistance, DNA binding, and subcellular localization. BMC Cancer 2012; 12:381
13. Zhong T, Xu F, Xu J, Liu L, Chen Y Aldo-keto reductase 1C3 (AKR1C3) is associated with the doxorubicin resistance in human breast cancer via PTEN loss. Biomed Pharmacother 2015; 69:317-25
14. Liu C, Lou W, Zhu Y, Yang J C, Nadiminty N, Gaikwad N W, et al. Intracrine Androgens and AKR1C3 Activation Confer Resistance to Enzalutamide in Prostate Cancer. Cancer Res 2015; 75:1413-22
15. Liu C, Armstrong C M, Lou W, Lombard A, Evans C P, Gao A C. Inhibition of AKR1C3 Activation Overcomes Resistance to Abiraterone in Advanced Prostate Cancer. Mol Cancer Ther 2017; 16:35-44
16. Zhao J, Xiang Y, Xiao C, Guo P, Wang D, Liu Y, et al. AKR1C3 overexpression mediates methotrexate resistance in choriocarcinoma cells. Int J Med Sci 2014; 11:1089-97
17. Xiong W, Zhao J, Yu H, Li X, Sun S, Li Y, et al. Elevated expression of AKR1C3 increases resistance of cancer cells to ionizing radiation via modulation of oxidative stress. PLoS One 2014; 9:e111911
18. Sun S Q, Gu X, Gao X S, Li Y, Yu H, Xiong W, et al. Overexpression of AKR1C3 significantly enhances human prostate cancer cells resistance to radiation. Oncotarget 2016; 7:48050-8
19. Xie L, Yu J, Guo W, Wei L, Liu Y, Wang X, et al. Aldo-keto reductase 1C3 may be a new radioresistance marker in non-small-cell lung cancer. Cancer Gene Ther 2013; 20:260-6
20. Ascierto M L, McMiller T L, Berger A E, Danilova L, Anders R A, Netto G J, et al. The Intratumoral Balance between Metabolic and Immunologic Gene Expression Is Associated with Anti-PD-1 Response in Patients with Renal Cell Carcinoma. Cancer Immunol Res 2016; 4:726-33
21. Moradi Manesh D, El-Hoss J, Evans K, Richmond J, Toscan C E, Bracken L S, et al. AKR1C3 is a biomarker of sensitivity to PR-104 in preclinical models of T-cell acute lymphoblastic leukemia. Blood 2015; 126:1193-202
22. Guise C P, Abbattista M R, Tipparaju S R, Lambie N K, Su J, Li D, et al. Diflavin oxidoreductases activate the bioreductive prodrug PR-104A under hypoxia. Mol Pharmacol 2012; 81:31-40
23. Guise C P, Wang A T, Theil A, Bridewell D J, Wilson W R, Patterson A V Identification of human reductases that activate the dinitrobenzamide mustard prodrug PR-104A: a role for NADPH:cytochrome P450 oxidoreductase under hypoxia. Biochem Pharmacol 2007; 74:810-20
24. Patterson A V, Ferry D M, Edmunds S J, Gu Y, Singleton R S, Patel K, et al. Mechanism of action and preclinical antitumor activity of the novel hypoxia-activated DNA cross-linking agent PR-104. Clin Cancer Res 2007; 13:3922-32
25. Evans K, Duan J, Pritchard T, Jones C D, McDermott L, Gu Z, et al. OBI-3424, a Novel AKR1C3-Activated Prodrug, Exhibits Potent Efficacy against Preclinical Models of T-ALL. Clin Cancer Res 2019; 25:4493-503
26. Wang Y, Liu Y, Zhou C, Wang C, Zhang N, Cao D, et al. An AKR1C3-specific prodrug with potent anti-tumor activities against T-ALL. Leuk Lymphoma 2020:1-9
27. Spiegelberg L, Houben R, Niemans R, de Ruysscher D, Yaromina A, Theys J, et al. Hypoxia-activated prodrugs and (lack of) clinical progress: The need for hypoxia-based biomarker patient selection in phase III clinical trials. Clin Transl Radiat Oncol 2019; 15:62-9
28. Heeke A L, Pishvaian M J, Lynce F, Xiu J, Brody J R, Chen W J, et al. Prevalence of Homologous Recombination-Related Gene Mutations Across Multiple Cancer Types. JCO Precis Oncol 2018; 2018
29. Ishida Y, Agata Y, Shibahara K, Honjo T, et al. Induced expression of PD-1, a novel member of the immunoglobulin gene superfamily, upon programmed cell death. EMBO J 1992; 11:3887-95
30. Agata Y, Kawasaki A, Nishimura H, Ishida Y, Tsubata T, Yagita H, Honjo T, et al. Expression of the PD-1 antigen on the surface of stimulated mouse T and B lymphocytes. Int Immunol 1996; 8:765-72
31. Freeman G J, Long A J, Iwai Y, Bourque K, Chernova T, Nishimura H, Fitz L J, Malenkovich N, Okazaki T, Byrne M C, Horton H F, Fouser L, Carter L, Ling V, Bowman M R, Carreno B M, Collins M, Wood C R, Honjo T, et al.
Engagement of the PD-1 immunoinhibitory receptor by a novel B7 family member leads to negative regulation of lymphocyte activation. J Exp Med. 2000; 192: 1027-34
32. Latchman Y, Wood C R, Chernova T, Chaudhary D, Borde M, Chernova I, Iwai Y, Long A J, Brown J A, Nunes R, Greenfield E A, Bourque K, Boussiotis V A, Carter L L, Carreno B M, Malenkovich N, Nishimura H, Okazaki T, Honjo T, Sharpe A H, Freeman G J, et al. PD-L2 is a second ligand for PD-1 and inhibits T cell activation. Nat Immunol 2001; 2:261-8.
33. Padmanee Sharma and James P Allison. The future of immune checkpoint therapy. Science 2015; 348:56-61.
34. Suzanne L Topalian, Janis M Taube, Robert A Anders, Drew M Pardoll, et al. Mechanism-driven biomarkers to guide immune checkpoint blockade in cancer therapy. Nat Rev Cancer 2016; 16:275-87.
35. Sofia Farkona, Eleftherios P Diamandis, Ivan M Blasutig, et al. Cancer immunotherapy: the beginning of the end of cancer? BMC Med 2016; 14:73.

1. A pharmaceutical composition, comprising:
(1) a compound 1-(3-(3-N,N-dimethylaminocarbonyl)phenoxyl-4-mtrophenyl)-1-ethyl-N,N′-bis(ethylene)phosphoramidate represented by Formula I, 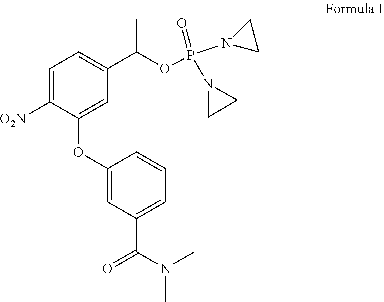 [SMILES: CC(OP(=O)(N1CC1)N1CC1)c1ccc([N+](=O)[O-])c(Oc2cccc(C(=O)N(C)C)c2)c1]
or a pharmaceutically acceptable salt, isotopic variant or solvate thereof; and
(2) at least one therapeutic agent including a chemotherapeutic agent or biological agent.

2. The pharmaceutical composition of claim 1, wherein the compound is (S)-1-(3-(3-N,N-dimethylaminocarbonyl)phenoxyl-4-mtrophenyl)-1-ethyl-N,N′-bis(ethylene)phosphoramidate represented by Formula I-1, or 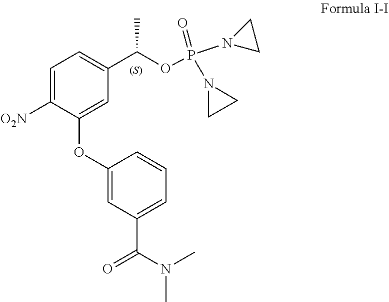
(R)-1-(3-(3-N,N-dimethylaminocarbonyl)phenoxyl-4-mtrophenyl)-1-ethyl-N,N′-bis(ethylene)phosphoramidate represented by Formula I-2

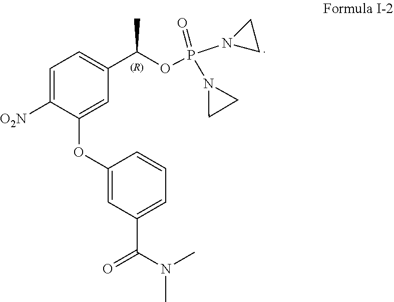

3. The pharmaceutical composition of claim 1, wherein the chemotherapeutic agent is selected from Monomethyl auristatin E (MMAE), Monomethyl auristatin F (MMAF), mertansine (DM1), anthracycline, pyrrolobenzodiazepine, α-amanitin, tubulysin, benzodiazepine, erlotinib, bortezomib, fulvestrant, sunitinib, letrozole, imatinib mesylate, PTK787/ZK 222584, oxaliplatin, leucovorin, rapamycin, lapatinib, lonafarnib, sorafenib, gefitinib, AG1478, AG1571, alkylating agent, alkyl sulfonate, aziridines, ethylenimine, methylamelamine, acetogenins, camptothecin, bryostatin, callystatin, CC-1065, cryptophycins, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, calicheamicin, dynemicin, clodronate, esperamicin, neocarzinostatin chromophore, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, epirubicin, esorubicin, idarubicin, marcellomycin, mitomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, epothilone, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, razoxane, rhizoxin, sizofiran, spirogermanium, tenuazonic acid, triaziquone, 2,2′,2″-trichlorotriethylamine, trichothecene, urethan, vindesine, dacarbazine, mannomustine, mitobronitol, mitolactol, pipobroman, gacytosine, arabinoside, cyclophosphamide, thiotepa, taxoid, paclitaxel, doxetaxel, chloranbucil, gemcitabine, 6-thioguanine, mercaptopurine, methotrexate, cisplatin, carboplatin, vinblastine, platinum, etoposide, ifosfamide, mitoxantrone, vincristine, vinorelbine, novantrone, teniposide, edatrexate, daunomycin, aminopterin, xeloda, ibandronate, topoisomerase inhibitor, difluoromethylornithine (DMFO), retinoid and capecitabine.

4. The pharmaceutical composition of claim 1, wherein the biological agent is selected from a peptide, protein, antibody, hormone, cytokine or chemokine.

5. The pharmaceutical composition of claim 4, wherein the antibody is an anti-immune checkpoint antibody which inhibits/blocks an inhibitory immune checkpoint antigen.

6. The pharmaceutical composition of claim 5, wherein the anti-immune checkpoint antibody is an anti-PD-1/PD-L1 antibody, anti-CTLA-4 antibody, anti-LAG-3 antibody, anti-TIGIT antibody, anti-Ceacam 1 antibody, anti-LAIR-1 antibody, anti-TIM-3 antibody, anti-VISTA antibody, anti-KIR antibody, anti-IDO antibody, anti-CD276 antibody, anti-A2AR antibody or anti-CD47 antibody.

7. The pharmaceutical composition of claim 6, wherein the anti-PD-1/PD-L1 antibody is avelumab, nivolumab, pembrolizumab, durvalumab and/or atezolizumab.

8. The pharmaceutical composition of claim 1, further comprising a pharmaceutically acceptable excipient.

9. Use of the pharmaceutical composition of claim 1 in the manufacture of a medicament for treating cancer in a patient.

10. The use of claim 9, wherein the cancer is AKR1C3 reductase overexpressing cancer.

11. The use of claim 9, wherein the cancer is liver cancer, hepatocellular carcinoma (HCC), lung cancer, melanoma, prostate cancer, breast cancer, leukemia, esophageal cancer, renal cancer, gastric cancer, colon cancer, brain cancer, bladder cancer, cervical cancer, ovarian cancer, head and neck cancer, endometrial cancer, pancreatic cancer, a sarcoma cancer, or rectal cancer.

12. A method for treating cancer in a patient in need thereof, comprising the step of administering to the patient a therapeutically effective amount of the pharmaceutical composition of claim 1.

13. The method of claim 12, wherein the therapeutically effective amount is from 0.1 mg/kg to 100 mg/kg.

14. The method of claim 12, wherein the cancer is AKR1C3 reductase overexpressing cancer.

15. The method of claim 12, wherein the cancer is liver cancer, hepatocellular carcinoma (HCC), lung cancer, melanoma, prostate cancer, breast cancer, leukemia, esophageal cancer, renal cancer, gastric cancer, colon cancer, brain cancer, bladder cancer, cervical cancer, ovarian cancer, head and neck cancer, endometrial cancer, pancreatic cancer, a sarcoma cancer, or rectal cancer.

16. A method for inhibiting the growth of cancer cells, comprising administering to a patient in need thereof a therapeutically effective amount of a pharmaceutical composition comprising a compound represented by Formula I-1 or Formula I-2 in combination with an immune checkpoint inhibitor

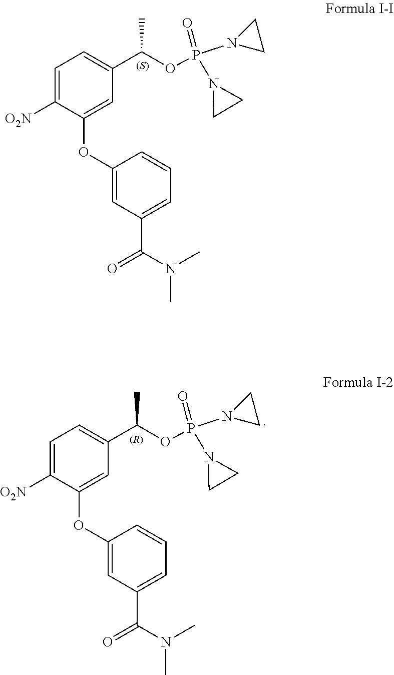

17. The method of claim 16, wherein the combination of the compound with the immune checkpoint inhibitor blockage acts corporately or synergistically to rescue a T cell inactivation and improve therapeutic efficacy.

18. The method of claim 16, wherein the immune checkpoint inhibitor is an anti-PD-1/PD-L1 antibody.

19. The method of claim 16, wherein the cancer is AKR1C3 reductase overexpressing cancer.

20. The method of claim 16, wherein the cancer is liver cancer, hepatocellular carcinoma (HCC), lung cancer, melanoma, prostate cancer, breast cancer, leukemia, esophageal cancer, renal cancer, gastric cancer, colon cancer, brain cancer, bladder cancer, cervical cancer, ovarian cancer, head and neck cancer, endometrial cancer, pancreatic cancer, a sarcoma cancer, or rectal cancer.
